(19) United States
(12) Patent Application Publication
(10) Pub. No.: US 2024/0200062 A1
(43) Pub. Date: Jun. 20, 2024

(54) METHODS OF TREATING DUCHENNE MUSCULAR DYSTROPHY USING PEPTIDE-OLIGONUCLEOTIDE CONJUGATES

(71) Applicant: PepGen Inc., Cambridge, MA (US)

(72) Inventors: Caroline GODFREY, Oxford (GB); Sonia BRACEGIRDLE, Belsele (BE); Ashling HOLLAND, Dublin (IE); Smita GUNNOO, Oxford (GB)

(21) Appl. No.: 18/281,680

(22) PCT Filed: Mar. 11, 2022
(86) PCT No.: PCT/US2022/020061

§ 371 Date: Sep. 12, 2023

Related U.S. Application Data
(60) Provisional application No. 63/194,039, filed on May 27, 2021.
(60) Provisional application No. 63/160,705, filed on Mar. 12, 2021.

(51) Int. Cl. C12N 15/113; A61K 9/00; A61K 47/64; A61P 21/00; C07K 7/08

(52) U.S. Cl. CPC C12N 15/113; A61K 9/0019; A61K 47/64; A61P 21/00; C07K 7/08; C12N 2310/11; C12N 2310/3233

(57) ABSTRACT

Disclosed are methods of treating a subject having Duchenne muscular dystrophy. The method includes administration of 1 mg/kg to 60 mg/kg of a conjugate of an oligonucleotide and a peptide covalently bonded or linked via a linker to the oligonucleotide to the subject (e.g., a subject amenable to exon 51 skipping). The peptide including at least one cationic domain including at least 4 amino acid residues and at least one hydrophobic domain including at least 3 amino acid residues, provided that the peptide includes a total of 7 to 40 amino acid residues, and provided that the at least one cationic domain includes a beta-alanine residue in combination with arginine and/or histidine residues. The oligonucleotide including a total of 12 to 40 contiguous nucleobases, wherein at least 12 contiguous nucleobases are complementary to a target sequence in a human dystrophin gene.

### FIELD OF THE INVENTION

The invention relates to peptide conjugates of antisense oligonucleotides, compositions containing them, and methods of their use.

### BACKGROUND

Nucleic acid drugs are genomic medicines with the potential to transform human healthcare. Research has indicated that such therapeutics could have applications across a broad range of disease areas including neuromuscular disease. The application of antisense oligonucleotide-based methods to modulate pre-mRNA splicing in the neuromuscular disease Duchenne muscular dystrophy (DMD) has placed this monogenic disorder at the forefront of advances in precision medicine.

However, therapeutic development of these promising antisense therapeutics has been hampered by insufficient cell-penetrance and poor distribution characteristics—a challenge that is further emphasized by the large volume and dispersed nature of the muscle tissue substrate in DMD.

DMD affects one in 3500 newborn boys. This severe, X-linked recessive disease results from mutations in the DMD gene, which encodes dystrophin protein. The disorder is characterized by progressive muscle degeneration and wasting, along with the emergence of respiratory failure and cardiac complications, ultimately leading to premature death. The majority of mutations underlying DMD are genomic out-of-frame deletions that induce a premature truncation in the open reading frame, which results in the absence of the dystrophin protein.

Exon skipping therapy utilizes splice switching antisense oligonucleotides (SSOs) to target specific regions of the DMD transcript, inducing the exclusion of individual exons, leading to the restoration of aberrant reading frames and resulting in the production of an internally deleted, yet partially functional, dystrophin protein. Despite the undoubted potential of antisense oligonucleotide exon skipping therapy for DMD, the successful application of this approach is currently limited by the relatively inefficient targeting of skeletal muscle, as well as the inadequate targeting of single stranded oligonucleotides to other affected tissues such as the heart. In September 2016 the Food and Drug Administration (FDA) granted accelerated approval for eteplirsen, a modulator of exon 51 splicing. Although this heralded the first US FDA-approved oligonucleotide that modulates splicing, the levels of dystrophin restoration were disappointing, with approximately 1% of normal dystrophin levels. Comparisons with the allelic disorder Becker muscular dystrophy (BMD) and experiments in the mdx mouse have indicated that homogenous sarcolemmal dystrophin expression of at least −15% of wild-type is needed to protect muscle against exercise induced damage.

Therefore, there is a need for new antisense oligonucleotide-based therapies for devastating genetic diseases such as DMD.

### SUMMARY OF THE INVENTION

In general, the invention provides methods of treating a subject having Duchenne muscular dystrophy.

In one aspect, the method includes administering 1 mg/kg to 60 mg/kg of a conjugate of an oligonucleotide and a peptide covalently bonded or linked via a linker to the oligonucleotide, the peptide including at least one cationic domain including at least 4 amino acid residues and at least one hydrophobic domain including at least 3 amino acid residues, provided that the peptide includes a total of 7 to 40 amino acid residues, and provided that the at least one cationic domain includes a beta-alanine residue in combination with arginine and/or histidine residues; and the oligonucleotide including a total of 12 to 40 contiguous nucleobases, where at least 12 contiguous nucleobases are complementary to a target sequence in a human dystrophin gene.

In some embodiments, the oligonucleotide includes the following sequence: 5′-CTCCAACATCAAGGAAGATGGCATTTCTAG-3′ (SEQ ID NO: 106). In some embodiments, the oligonucleotide includes a sequence selected from the group consisting of:

| SEQ | |
|---|---|
| ID | |
| NO: | Sequence |
| 107 | 5′-ACCAGAGUAACAGUCUGAGUAGGAGC-3′ |
| 108 | 5′-CUCAUACCUUCUGCUUGAUGAUC-3′ |
| 109 | 5′-UUCUGUCCAAGCCCGGUUGAAAUC-3′ |
| 110 | 5′-ACAUCAAGGAAGAUGGCAUUUCUAGUUUGG-3′ |
| 111 | 5′-ACAUCAAGGAAGAUGGCAUUUCUAG-3′ |
| 112 | 5′-CUCCAACAUCAAGGAAGAUGGCAUUUCUAG-3′ |
| 113 | 5′-AUCAUUUUUUCUCAUACCUUCUGCUAG-3′ |
| 114 | 5′-AUCAUUUUUUCUCAUACCUUCUGCUAGGAG |
| | CUAAAAAG-3′ |
| 115 | 5′-CACCCACCAUCACCCUCUGUG-3′ |
| 116 | 5′-AUCAUCUCGUUGAUAUCCUCAA-3′ |

and their thymine-substitution analogues.

In some embodiments, each cationic domain has length of between 4 and 12 amino acid residues. In some embodiments, each cationic domain has length of between 4 and 7 amino acid residues. In some embodiments, each cationic domain includes at least 55%, at least 60%, at least 65% at least 70%, at least 75%, at least 80%, at least 85%, at least 90%, or at least 95% cationic amino acids. In some embodiments, each cationic domain includes at least 40%, at least 45%, at least 50%, at least 55%, at least 60%, at least 60%, at least 65%, at least 70% arginine and/or histidine residues.

In some embodiments, each cationic domain includes one of the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19), or any combination thereof. In some embodiments, each cationic domain comprises or consists of one the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19), or any combination thereof.

In some embodiments, the peptide includes two cationic domains.

In some embodiments, each hydrophobic domain has a length of 3 to 6 amino acids, preferably each hydrophobic domain has a length of 5 amino acids. In some embodiments, each hydrophobic domain includes at least 70%, at least 75%, at least 80%, at least 85%, at least 90%, at least 95%, or 100% hydrophobic amino acids. In some embodiments, each hydrophobic domain includes phenylalanine, leucine, Isoleucine, tyrosine, tryptophan, proline, and/or glutamine residues; preferably where each hydrophobic domain comprises or consists of phenylalanine, leucine, isoleucine, tyrosine, tryptophan, proline, and/or glutamine residues. In some embodiments, the peptide includes one hydrophobic domain. In some embodiments, the or each hydrophobic domain includes one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26) or any combination thereof. In some embodiments, the or each hydrophobic domain comprises or consists of one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26) or any combination thereof.

In some embodiments, the peptide comprises or consists of two cationic domains and one hydrophobic domain. In some embodiments, the peptide comprises or consists of one hydrophobic core domain flanked by two cationic arm domains.

In some embodiments, the peptide comprises or consists of one hydrophobic core domain including a sequence selected from: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26), flanked by two cationic arm domains each including a sequence selected from: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19).

In some embodiments, the peptide comprises or consists of one of the following sequences:

(SEQ ID NO: 27)
RBRRBRRFQILYRBRBR,
(SEQ ID NO: 31)
RBRRBRRYQFLIRBRBR,
(SEQ ID NO: 32)
RBRRBRRILFQYRBRBR,
(SEQ ID NO: 35)
RBRRBRFQILYBRBR,
(SEQ ID NO: 37)
RBRRBRRFQILYRBHBH,
(SEQ ID NO: 38)
RBRRBRRFQILYHBHBR,
and
(SEQ ID NO: 44)
RBRRBRFQILYRBHBH

In some embodiments, the peptide comprises or has the following amino acid sequence RBRRBRFQILYBRBR (SEQ ID NO: 35). In some embodiments, the peptide comprises or has the following amino acid sequence RBRRBRRFQILYRBHBH (SEQ ID NO: 37). In some embodiments, the peptide comprises or has the following amino acid sequence RBRRBRFQILYRBHBH (SEQ ID NO: 44).

In some embodiments, the peptide is bonded to the rest of the conjugate through its N-terminus. In some embodiments, the C-terminus of the peptide is —NH₂. In some embodiments, the peptide is bonded to the rest of the conjugate through its C-terminus. In some embodiments, the peptide is acylated at its N-terminus.

In some embodiments, the conjugate comprises or is of the following structure:

[peptide]-[linker]-[oligonucleotide]

In some embodiments, the conjugate comprises or is of the following structure:

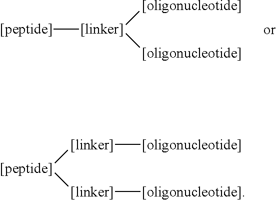

In some embodiments, the conjugate comprises or is of the following structure:

[peptide]-[linker]-[peptide]-[linker]-[oligonucleotide].

In some embodiments, each linker is independently of formula (I):

T₁—(CR¹R²)ₙ—T₂. (I)

where

T₁ is a divalent group for attachment to the peptide and is selected from the group consisting of —NH— and carbonyl;

T₂ is a divalent group for attachment to an oligonucleotide and is selected from the group consisting of —NH— and carbonyl;

n is 1, 2 or 3;

each R¹ is independently —Y¹—X¹—Z¹, where
Y¹ is absent or —(CR^{A1}R^{A2})ₘ—, where m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —N(R^{A3})C(NR^{A3})N(R^{A3})—, —SO—, —S—, —SO₂—, —S(O)₂N(R^{A3})—, or —N(R^{A3})SO₂—, where each R^{A3} is independently selected from hydrogen and methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl,

where each (1-6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4} R^{A5}, and (1-4C)alkoxy, where R^{A4} and R^{A5} are each independently selected from the group consisting of hydrogen and (1-4C)alkyl; and

each R² is independently —Y²—X²—Z², where
Y² is absent or a group of the formula —[CR^{B1} R^{B2}]ₘ— in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})—, or —N(R^{B3})SO₂—, where each R^{B3} is independently selected from hydrogen or methyl; and
Z² is selected from hydrogen, (1-6C)alkyl, (2-6C)alkenyl, (2-6C)alkynyl, aryl, (3-6C)cycloalkyl, (3-6C)cycloalkenyl or heteroaryl, where each (1-6C)alkyl, (2-6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4} R^{B5}, and (1-4C)alkoxy, where R^{B4} and R^{B5} are each independently hydrogen or (1-2C)alkyl; with the proviso that; when n=1 and T₁ and T₂ are different to one another, then R¹ and R² are not both H; when n=1, T₁ and T₂ are different to one another and one of R¹ and R² is H then the other of R¹ and R² is not methyl; or when n=2 and each occurrence of R¹ and R² is H, then T₁ and T₂ are both —C(O)— or are both —NH—.

In some embodiments, T₂ is —C(O)—.

In some embodiments, each R¹ is independently —Y¹ —X¹ —Z¹, where:

Y¹is absent or —(CR^{A1}R^{A2})ₘ—, where m is 1, 2, 3 or 4, and R^{A1} and R^{A2} are each hydrogen or (1-2C)alkyl;

X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(N R^{A3})N(R^{A3})— or —S—, where each R^{A3} is independently hydrogen or methyl; and

Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl, where each (1-6C)alkyl, (2-6C)alkenyl, (2-6C)alkynyl, aryl, (3-6C)cycloalkyl, (3-6C)cycloalkenyl, and heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{A4} R^{A5}, and (1-4C)alkoxy, where RM and R^{A5} are each independently hydrogen or (1-2C)alkyl.

In some embodiments, each R¹ is independently —Y¹ —X¹ —Z¹, where:

Y¹is absent or —(CR^{A1}R^{A2})ₘ—, where m is 1, 2, 3, or 4, and R^{A1} and R″ are each independently hydrogen or (1-2C)alkyl;

X¹ is absent, —O—, —C(O)—, —C(O)O—, —N(R^{A3})—, —N(R^{A3})—C(O)—, —C(O)—N (R^{A3})—, —N(R^{A3})C(O)N(R^{A3})—, —N(R^{A3})C(NR^{A3})N(R^{A3})—, or —S—, where each R^{A3} is independently hydrogen or methyl; and

Z¹ is a further oligonucleotide or is hydrogen, (1-6C)alkyl, aryl, (3-6C)cycloalkyl, or heteroaryl, where each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

In some embodiments, each R¹ is independently —Y¹ —X¹ —Z¹, where:

Y¹is absent or a group of the formula —(CR^{A1}R^{A2})ₘ—, where m is 1, 2, 3 or 4, and R^{A1} and R^{A2} are each independently hydrogen or (1-2C)alkyl;

X¹ is absent, —C(O)—, —C(O)O—, —N(R^{A3})—C(O)—, —C(O)—N(R^{A3})—, where each R^{A3} is hydrogen or methyl; and

Z¹ is a further oligonucleotide or is hydrogen, (1-6C)alkyl, aryl, (3-6C)cycloalkyl, or heteroaryl, where each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

In some embodiments, each R¹ is independently —Y¹ —X¹ —Z¹, where:

Y¹ is absent, —(CH₂)—, or —(CH₂CH₂)—;

X¹ is absent, —N(R^{A3})—C(O)—, —C(O)—N(R^{A3})—, where each R^{A3} is independently hydrogen or methyl; and

Z¹ is hydrogen or (1-2C)alkyl.

In some embodiments, each R² is independently —Y²—Z²,

where Y² is absent or —(CR^{B1}R^{B2}

R^{B2})ₘ—, where m is 1, 2, 3 or 4, and R^{B1} and R^{B2} are each independently hydrogen or (1-2C)alkyl; and

Z² is hydrogen or (1-6C)alkyl.

In some embodiments, each R² is hydrogen.

In some embodiments, n is 2 or 3. In some embodiments, n is 1.

In some embodiments, the linker is an amino acid residue selected from the group consisting of glutamic acid, succinic acid, and gamma-aminobutyric acid residues.

In some embodiments, the linker comprises or is of the following structure:

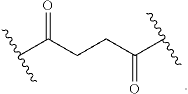 [SMILES: *C(=O)CCC(*)=O]

In some embodiments, the linker comprises or is of the following structure:

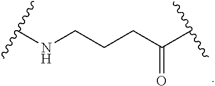 [SMILES: *NCCCC(*)=O]

In some embodiments, the linker comprises or is of the following structure:

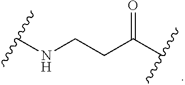 [SMILES: *NCCC(*)=O]

In some embodiments, the linker comprises or is of the following structure:

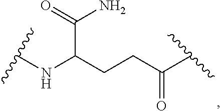 [SMILES: *NC(CCC(*)=O)C(N)=O]

In some embodiments, the linker comprises or is of the following structure:

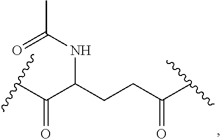 [SMILES: *C(=O)CCC(NC(C)=O)C(*)=O]

In some embodiments, the conjugate comprises or is of the following structure:

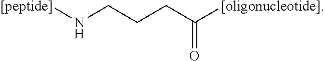

In some embodiments, the conjugate comprises or is of the following structure:

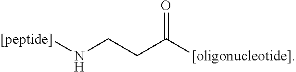

In some embodiments, the conjugate comprises or is of the following structure:

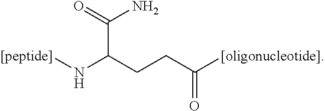

In some embodiments, the conjugate comprises or is of the following structure:

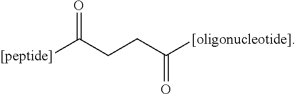

In some embodiments, the conjugate comprises or is of the following structure:

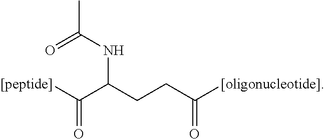

In some embodiments, the oligonucleotide is bonded to the linker or the peptide at its 3′ terminus.

In some embodiments, the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂. In some embodiments, the conjugate has the structure:

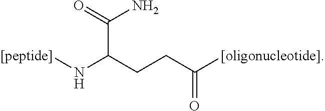

In some embodiments, the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYBRBR, where free —COOH, if any, in the glutamic acid residue is replaced with —CONH₂. In some embodiments, the conjugate comprises or consists of 5′-UUCUGUCCAAGCCCGGUUGAAAUC-3′ (SEQ ID NO: 109), ora thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂. In some embodiments, the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂. In some embodiments of this paragraph, the conjugate has the structure:

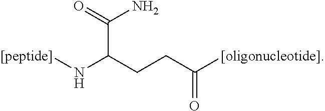

In some embodiments, the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂. In some embodiments, the conjugate has the structure:

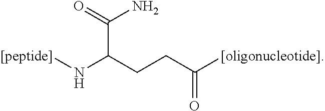

In some embodiments, the conjugate comprises or consists of 5′-CTCCAACATCAAGGAAGATGGCATTTCTAG-3′ (SEQ ID NO: 106) having a 3′-terminus covalently linked via a glutamic acid residue to N-terminus of peptide RBRRBRFQILYBRBR—NH₂, where free —COOH, if any, in the glutamic acid residue is replaced with —CONH₂. In some embodiments, the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂. In some embodiments, the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂. In some embodiments of this paragraph, the conjugate has the structure:

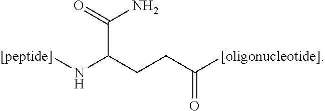

In some embodiments, the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via gamma-aminobutyric acid residue to C-terminus of peptide Ac-RBRRBRFQILYRBHBH.

In some embodiments, the conjugate comprises or consists of 5′-CTCCAACATCAAGGAAGATGGCATTTCTAG-3′ (SEQ ID NO: 106) having a 3′-terminus covalently linked via gamma-aminobutyric acid residue to C-terminus of peptide Ac-RBRRBRFQILYRBHBH. In some embodiments, the conjugate comprises or consists of 5′-UUCUGUCCAAGCCCGGUUGAAAUC-3′ (SEQ ID NO: 109), ora thymine-substitution analogue thereof, having a 3′-terminus covalently linked via gamma-aminobutyric acid residue to C-terminus of peptide Ac-RBRRBRFQILYRBHBH. In some embodiments, the conjugate comprises or consists of 5′-CACCCACCAUCACCCUCUGUG-3′ (SEQ ID NO: 115), or a thymine-substitution analogue thereof, having a 3′-terminus covalently linked via gamma-aminobutyric acid residue to C-terminus of peptide Ac-RBRRBRFQILYRBHBH.

In some embodiments, the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG- 3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′- AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to C-terminus of peptide Ac-RBRRBRFQILYRBHBH, wherein free —COOH, if any, in the glutamic acid residue is replaced with —CONH₂. In some embodiments, the conjugate has the structure:

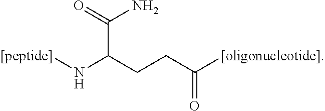

In some embodiments, the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYRBHBH, where free —COOH, if any, in the glutamic acid residue is replaced with —CONH₂. In some embodiments, the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to C-terminus of peptide Ac-RBRRBRFQILYRBHBH, wherein free —COOH, if any, in the glutamic acid residue is replaced with —CONH₂. In some embodiments, the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to C-terminus of peptide Ac-RBRRBRFQILYRBHBH, wherein free —COON, if any, in the glutamic acid residue is replaced with —CONH₂. In some embodiments of this paragraph, the conjugate has the structure:

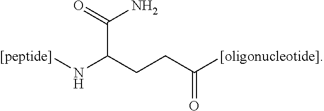

In some embodiments, the linker is of the following structure:

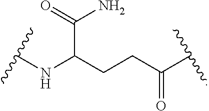

In some embodiments, the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a beta-alanine residue to C-terminus of peptide Ac-RBRRBRFQILYBRBR.

In some embodiments, the conjugate comprises or consists of 5′-CTCCAACATCAAGGAAGATGGCATTTCTAG-3′ (SEQ ID NO: 106) having a 3′-terminus covalently linked via a beta-alanine residue to C-terminus of peptide Ac-RBRRBRFQILYBRBR. In some embodiments, the conjugate comprises or consists of 5′-UUCUGUCCAAGCCCGGUUGAAAUC-3′ (SEQ ID NO: 109), or a thymine-substitution analogue thereof, having a 3′-terminus covalently linked via a beta-alanine residue to C-terminus of peptide Ac-RBRRBRFQILYBRBR. In some embodiments, the conjugate comprises or consists of 5′-CACCCACCAUCACCCUCUGUG-3′ (SEQ ID NO: 115), or a thymine-substitution analogue thereof, having a 3′-terminus covalently linked via a beta-alanine residue to C-terminus of peptide Ac-RBRRBRFQILYBRBR.

In some embodiments, the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a beta-alanine residue to C-terminus of peptide Ac-RBRRBRFQILYRBHBH.

In some embodiments, the conjugate comprises or consists of 5′-CTCCAACATCAAGGAAGATGGCATTTCTAG-3′ (SEQ ID NO: 106) having a 3′-terminus covalently linked via a beta-alanine residue to C-terminus of peptide Ac-RBRRBRFQILYRBHBH. In some embodiments, the conjugate comprises or consists of 5′-UUCUGUCCAAGCCCGGUUGAAAUC-3′ (SEQ ID NO: 109), or a thymine-substitution analogue thereof, having a 3′-terminus covalently linked via a beta-alanine residue to C-terminus of peptide Ac-RBRRBRFQILYRBHBH. In some embodiments, the conjugate comprises or consists of 5′-CACCCACCAUCACCCUCUGUG-3′ (SEQ ID NO: 115), or a thymine-substitution analogue thereof, having a 3′-terminus covalently linked via a beta-alanine residue to C-terminus of peptide Ac-RBRRBRFQILYRBHBH.

In some embodiments, the oligonucleotide is a morpholino.

In some embodiments, all morpholino internucleoside linkages are —P(O)(NMe₂)O—.

In some embodiments, the oligonucleotide comprises the following group as its 5′ terminus:

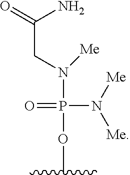 [SMILES: *OP(=O)(N(C)C)N(C)CC(N)=O]

In some embodiments of the above and other oligonucleotides described herein, the oligonucleotide is a phosphorodiamidate morpholino oligomer (PMO).

In some embodiments, the conjugate is administered parenterally. In some embodiments, the conjugate is administered by infusion (e.g., intravenous infusion, which optionally is bolus infusion or continuous infusion, with that latter optionally being for 10 minutes to 3 hours, e.g., 0.25-2 hours, or 0.5-1 hour). In some embodiments, the conjugate is administered by intravenous infusion. In some embodiments, the subject is amenable to exon 51 skipping.

In some embodiments, the conjugate is administered to the subject at a frequency that is weekly to monthly. In some embodiments, the frequency is weekly, biweekly, every three weeks, or monthly. In some embodiments, the frequency is quarterly.

In some embodiments, 40 mg/kg to 60 mg/kg, 30 mg/kg to 50 mg/kg, 30 mg/kg to 40 mg/kg, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, 45 mg/kg to 60 mg/kg, 1 mg/kg to 30 mg/kg, 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of conjugate is administered.

In some embodiments, 1 mg/kg, 4 mg/kg, 5 mg/kg, 6 mg/kg, 8 mg/kg, 10 mg/kg, 15 mg/kg, 20 mg/kg, 25 mg/kg, 30 mg/kg, 40 mg/kg, 50 mg/kg, or 60 mg/kg of conjugate is administered. In some embodiments, 1 mg/kg, 5 mg/kg, 10 mg/kg, 15 mg/kg, or 20 mg/kg of conjugate is administered.

In some embodiments, the method is for use in treating a cardiac effect of DMD including, e.g., cardiomyopathy (e.g., dilated, hypertrophic, or restrictive cardiomyopathy), heart failure, and/or cardiac arrhythmias.

In some embodiments, the methods described above and elsewhere herein are used in the treatment of BMD.

The invention also includes the use of the conjugates described herein in the methods described herein. Accordingly, each method of treatment claim herein can be considered as supporting a claim in the form of a composition as specified therein for use in the indicated method (e.g., the treatment, prevention, or amelioration of DMD or BMD).

### Definitions

References to “X” throughout denote any form of the amino acid aminohexanoic acid, such as 6-aminohexanoic acid.

References to “B” throughout denote the amino acid beta-alanine.

References to “[Hyp]” throughout denote the amino acid hydroxyproline.

References to “Ac” throughout denote an acetyl group (CH₃—C(O)—).

References to other capital letters throughout denote the relevant genetically encoded amino acid residue in accordance with the accepted alphabetic amino acid code.

The term “alkyl,” as used herein, refers to a straight or branched chain hydrocarbon group containing a total of one to twenty carbon atoms, unless otherwise specified (e.g., (1-6C) alkyl, (1-4C) alkyl, (1-3C) alkyl, or (1-2C) alkyl). Non-limiting examples of alkyls include methyl, ethyl, 1-methylethyl, propyl, 1-methylbutyl, 1-ethylbutyl, etc. References to individual alkyl groups such as “propyl” are specific for the straight chain version only, and references to individual branched chain alkyl groups such as “isopropyl” are specific for the branched chain version only.

The term “alkenyl”, as used herein, refers to an aliphatic group containing having one, two, or three carbon-carbon double bonds and containing a total of two to twenty carbon atoms, unless otherwise specified (e.g., (2-6C) alkenyl, (2-4C) alkenyl, or (2-3C) alkenyl). Non-limiting examples of alkenyl include vinyl, allyl, homoallyl, isoprenyl, etc. Unless otherwise specified, alkenyl may be optionally substituted by one, two, three, four, or five groups selected from the group consisting of carbocyclyl, aryl, heterocyclyl, heteroaryl, oxo, halogen, and hydroxyl.

The term “alkynyl”, as used herein, refers to an aliphatic group containing one, two, or three carbon-carbon triple bonds and containing a total of two to twenty carbon atoms, unless otherwise specified (e.g., (2-6C) alkynyl, (2-4C) alkynyl, or (2-3C) alkynyl). Non-limiting examples of alkynyl include ethynyl, propargyl, homopropargyl, but-2-yn-1-yl, 2-methyl-prop-2-yn-1-yl, etc. Unless otherwise specified, alkynyl may be optionally substituted by one, two, three, four, or five groups selected from the group consisting of carbocyclyl, aryl, heterocyclyl, heteroaryl, oxo, halogen, and hydroxyl.

By “arginine rich” with respect to a cationic domain is meant that at least 40% of the cationic domain is formed of arginine residues.

The term “artificial amino acid,” as used herein, refers to an abiogenic amino acid (e.g., non-proteinogenic). For example, artificial amino acids may include synthetic amino acids, modified amino acids (e.g., those modified with sugars), non-natural amino acids, man-made amino acids, spacers, and non-peptide bonded spacers. Synthetic amino acids may be those that are chemically synthesized by man. For the avoidance of doubt, aminohexanoic acid (X) is an artificial amino acid in the context of the present invention. For the avoidance of doubt, beta-alanine (B) and hydroxyproline (Hyp) occur in nature and therefore are not artificial amino acids in the context of the present invention but are natural amino acids. Artificial amino acids may include, for example, 6-aminohexanoic acid (X), tetrahydroisoquinoline-3-carboxylic acid (TIC), 1-(amino)cyclohexanecarboxylic acid (Cy), 3-azetidine-carboxylic acid (Az), and 11-aminoundecanoic acid.

The term “aryl,” as used herein, refers to a carbocyclic ring system containing one, two, or three rings, at least one of which is aromatic. An unsubstituted aryl contains a total of 6 to 14 carbon atoms.

The term aryl includes both monovalent species and divalent species. Examples of aryl groups include, but are not limited to, phenyl, naphthyl, indanyl, and the like. In particular embodiments, an optionally substituted aryl is optionally substituted phenyl.

By “bridged ring systems,” as used herein, are meant ring systems in which two rings share more than two atoms, see for example Advanced Organic Chemistry, by Jerry March, 4th Edition, Wiley Interscience, pages 131-133, 1992. Examples of bridged heterocyclyl ring systems include, aza-bicydo[2.2.1]heptane, 2-oxa-5-azabicyclo[2.2.1]heptane, aza-bicyclo[2.2.2]octane, aza-bicyclo[3.2.1]octane, quinuclidine, etc.

The term “carbonyl,” as used herein, refers to a group of the following structure —C(O)—. Non-limiting examples of carbonyl groups include those found, e.g., in acetone, ethyl acetate, proteinogenic amino acids, acetamide, etc.

References made herein to “cationic” denote an amino acid or domain of amino acids having an overall positive charge at physiological pH.

The term “(m-nC)” or “(m-nC) group” used alone or as a prefix, refers to a group having a total of m to n carbon atoms, when unsubstituted.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The term “cycloalkyl,” as used herein, refers to a saturated carbocyclic ring system containing one or two rings, and containing a total of 3 to 10 carbon atoms, unless otherwise specified. The two-ring cycloalkyls may be arranged as fused ring systems (two bridgehead carbon atoms are directly bonded to one another), bridged ring systems (two bridgehead carbon atoms are linked to one another via a covalent linker containing at least one carbon atom), and spiro-ring (two rings are fused at the same cabron atom) systems. Non-limiting examples of cycloalkyl include cyclopropyl, cyclobutyl, cyclopentyl, cyclohexyl, cycloheptyl, bicyclo[2.2.1]heptyl, etc.

The term “cycloalkenyl,” as used herein, refers to a non-aromatic, unsaturated, carbocyclic ring system containing one or two rings; containing one, two, or three endocyclic double bonds; and containing a total of 3 to 10 carbon atoms, unless otherwise specified. The two-ring cycloalkenyls may be arranged as fused ring systems (two bridgehead carbon atoms are directly bonded to one another), bridged ring systems (two bridgehead carbon atoms are linked to one another via a covalent linker containing at least one carbon atom), and spiro-ring (two rings are fused at the same cabron atom) systems. Non-limiting examples of cycloalkenyl include cyclobutenyl, cyclopentenyl, cyclohexenyl, cycloheptenyl, 3-cyclohexen-1-yl, cyclooctenyl, etc.

“Dystrophin” is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about 14-240 amino acids and a central rod domain at about 253-3040 amino acids. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, alpha-dystrobrevin, sarcospan, laminin, syntrophins, integrins, and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne's muscular dystrophy (DMD) and Beckers muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain subjects with DMD or BMD, as known in the art, or by the absence of detectable dystrophin.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 75 exons.

“Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein. In certain embodiments, the exon being skipped is an aberrant exon from the human dystrophin gene, which may contain a mutation or other alteration in its sequence that otherwise causes aberrant splicing. As described herein, the exon being skipped is exon 51 of the human dystrophin gene.

The term “halo” or “halogeno,” as used herein, refer to fluoro, chloro, bromo, and iodo.

By “histidine rich” with respect to a cationic domain it is meant that at least 40% of the cationic domain is formed of histidine residues.

The terms “heteroaryl” or “heteroaromatic,” as used interchangeably herein, refer to a ring system containing one, two, or three rings, at least one of which is aromatic and containing one to four (e.g., one, two, or three) heteroatoms selected from the group consisting of nitrogen, oxygen, and sulfur. An unsubstituted heteroaryl group contains a total of one to nine carbon atoms. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example, a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example, a single heteroatom. In some embodiments, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1 H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-13][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1.2.3.4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl,1.2.3.4-tetrahydropyrido[2,3-b]pyrazinyl and 3,4-dihydro-2W-pyrido[3,2-13][1,4]oxazinyl. Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups. Examples of six membered heteroaryl groups include but are not limited to pyridyl, pyrazinyl, pyridazinyl, pyrimidinyl and triazinyl. A bicyclic heteroaryl group may be, for example, a group selected from: a benzene ring fused to a 5- or 6-membered ring containing 1, 2 or 3 ring heteroatoms; a pyridine ring fused to a 5- or 6-membered ring containing 1, 2 or 3 ring heteroatoms; a pyrimidine ring fused to a 5- or 6-membered ring containing 1 or 2 ring heteroatoms; a pyrrole ring fused to a 5- or 6-membered ring containing 1, 2 or 3 ring heteroatoms; a pyrazole ring fused to a 5- or 6-membered ring containing 1 or 2 ring heteroatoms; a pyrazine ring fused to a 5- or 6-membered ring containing 1 or 2 ring heteroatoms; an imidazole ring fused to a 5- or 6-membered ring containing 1 or 2 ring heteroatoms; an oxazole ring fused to a 5- or 6-membered ring containing 1 or 2 ring heteroatoms; an isoxazole ring fused to a 5- or 6-membered ring containing 1 or 2 ring heteroatoms; a thiazole ring fused to a 5- or 6-membered ring containing 1 or 2 ring heteroatoms; an isothiazole ring fused to a 5- or 6-membered ring containing 1 or 2 ring heteroatoms; a thiophene ring fused to a 5- or 6-membered ring containing 1, 2 or 3 ring heteroatoms; a furan ring fused to a 5- or 6-membered ring containing 1, 2 or 3 ring heteroatoms; a cyclohexyl ring fused to a 5- or 6-membered heteroaromatic ring containing 1, 2 or 3 ring heteroatoms; and a cyclopentyl ring fused to a 5- or 6-membered heteroaromatic ring containing 1, 2 or 3 ring heteroatoms. Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups. Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The terms “heterocyclyl,” as used herein, refer to a ring system containing one, two, or three rings, at least one of which containing one to four (e.g., one, two, or three) heteroatoms selected from the group consisting of nitrogen, oxygen, and sulfur, provided that the ring system does not contain aromatic rings that also include an endocyclic heteroatom. An unsubstituted heterocyclyl group contains a total of two to nine carbon atoms. The term heterocyclyl includes both monovalent species and divalent species. Examples of heterocyclyl groups are monocyclic and bicyclic groups containing from five to twelve ring members, and more usually from five to ten ring members. The heterocyclyl group can be, for example, a 5- or 6-membered monocyclic ring or a 9- or 10-membered bicyclic ring, for example, a bicyclic structure formed from fused five and six membered rings or two fused six membered rings. Each ring may contain up to about four heteroatoms typically selected from nitrogen, sulfur and oxygen. Non-limiting examples of heterocyclyl groups include, e.g., pyrrolidine, piperazine, piperidine, azepane, 1,4-diazepane, tetrahydrofuran, tetrahydropyran, oxepane, 1,4-dioxepane, tetrahydrothiophene, tetrahydrothiopyran, indoline, benzopyrrolidine, 2,3-dihydrobenzofuran, phthalan, isochroman, and 2,3-dihydrobenzothiophene.

The term “internucleoside linkage,” as used herein, represents a group or bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non- limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

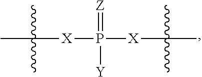

where

Z is O, S, or Se;

Y is —X—L—R¹;

each X is independently —O—,—S—, —N(—L—R¹)—, or L;

each L is independently a covalent bond or a linker (e.g., optionally substituted C₁₋₆₀ aliphatic linker or optionally substituted C₂₋₆₀ heteroaliphatic linker);

each R¹ is independently hydrogen, —S—S—R², —O—CO—R², —S—CO—R², optionally substituted C₁₋₉ heterocyclyl, or a hydrophobic moiety; and

each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl. When L is a covalent bond, R¹ is hydrogen, Z is oxygen, and all X groups are —O—, the internucleoside group is known as a phosphate phosphodiester. When L is a covalent bond, R¹ is hydrogen, Z is sulfur, and all X groups are —O—, the internucleoside group is known as a phosphorothioate diester. When Z is oxygen, all X groups are —O—, and either (1) L is a linker or (2) R¹ is not a hydrogen, the internucleoside group is known as a phosphotriester. When Z is sulfur, all X groups are —O—, and either (1) L is a linker or (2) R¹ is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

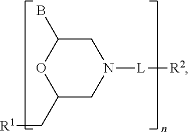

where

n is an integer of at least 10 (e.g., 12 to 30) indicating the number of morpholino subunits and associated groups L;

each B is independently a nucleobase;

R¹ is a 5′ group (R¹ may be referred to herein as a 5′ terminus);

R² is a 3′ group (R² may be referred to herein as a 3′ terminus); and

L is (i) a morpholino internucleoside linkage or, (ii) if L is attached to R², a covalent bond. A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a bond to a peptide, a bond to a peptide/linker combination, an endosomal escape moiety, or a neutral organic polymer. In some embodiments, the 5′ group is of the following structure:

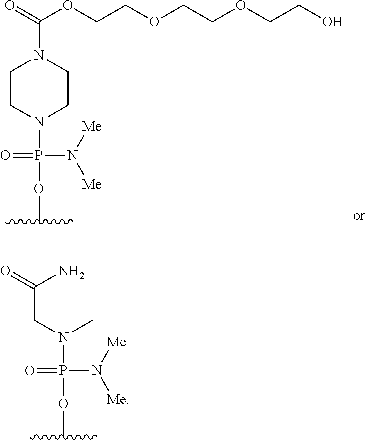 [SMILES: *OP(=O)(N(C)C)N1CCN(C(=O)OCCOCCOCCO)CC1 ; *OP(=O)(N(C)C)N(C)CC(N)=O]

Preferred 5′ group are hydroxyl and groups of the following structure:

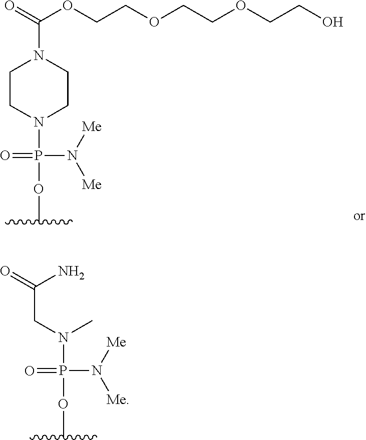 [SMILES: *OP(=O)(N(C)C)N1CCN(C(=O)OCCOCCOCCO)CC1 ; *OP(=O)(N(C)C)N(C)CC(N)=O]

A more preferred 5′ group is of the following structure:

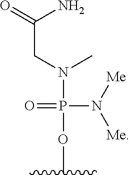 [SMILES: *OP(=O)(N(C)C)N(C)CC(N)=O]

A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a bond to a peptide, a bond to a peptide/linker combination, an endosomal escape moiety, or a neutral organic polymer. In a conjugate of an oligonucleotide that is a morpholino and a peptide that is covalently bonded or linked to the oligonucleotide, the preferred 3′ group is a bond to a peptide or a bond to a peptide/linker combination.

The term “morpholino internucleoside linkage,” as used herein, represents a divalent group of the following structure:

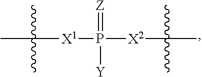

where

Z is O or S;

X¹ is a bond, —CH₂—, or —O—;

X² is a bond, —CH₂—O—, or —O—; and

Y is —NR₂, where each R is independently H or C₁₋₆ alkyl (e.g., methyl), or both R combine together with the nitrogen atom to which they are attached to form a C₂₋₉ heterocyclyl (e.g., N-piperazinyl); provided that both X¹ and X² are not simultaneously a bond.

The term “morpholino subunit,” as used herein, refers to the following structure:

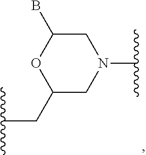

where B is a nucleobase.

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and O -6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2—, N6—, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6—N-benzoyladenine, 2—N-isobutyrylguanine, 4—N-benzoylcytosine, 4—N-benzoyluracil, 5-methyl 4—N-benzoylcytosine, 5-methyl 4—N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art, as well as modified or unmodified 2′-deoxyribofuranrpose-nucleobase compounds and groups known in the art. The sugar may be ribofuranose. The sugar may be modified or unmodified. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

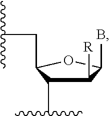

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides. A “nucleoside” may also refer to a morpholino subunit.

The term “nucleotide,” as used herein, represents a nucleoside bonded to an internucleoside linkage or a monovalent group of the following structure —X¹—P(X²)(R¹)2, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more contiguous nucleosides covalently bound together by internucleoside linkages; a morpholino containing or more morpholino subunits; or a peptide nucleic acid containing 10 or more morpholino subunits. Preferably, an oligonucleotide is a morpholino.

The term “optionally substituted” refers to groups, structures, or molecules that may be substituted or unsubstituted as described for each respective group. The term “wherein a/any CH, CH₂, CH₃ group or heteroatom (i.e., NH) within a R¹ group is optionally substituted” means that (any) one of the hydrogen radicals of the R¹ group is substituted by a relevant stipulated group.

In this specification the term “operably linked” may include the situation where a selected nucleotide sequence and regulatory nucleotide sequence are covalently linked in such a way as to place the expression of a nucleotide coding sequence under the control of the regulatory sequence, as such, the regulatory sequence is capable of effecting transcription of a nucleotide coding sequence which forms part or all of the selected nucleotide sequence. Where appropriate, the resulting transcript may then be translated into a desired peptide.

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “pharmaceutical composition,” as used herein, represents a composition containing an oligonucleotide described herein, formulated with a pharmaceutically acceptable excipient, and manufactured or sold with the approval of a governmental regulatory agency as part of a therapeutic regimen for the treatment of disease in a subject.

The term “pharmaceutically acceptable salt,” as used herein, means any pharmaceutically acceptable salt of a conjugate, oligonucleotide, or peptide disclosed herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “reduce” or “inhibit” may relate generally to the ability of one or more compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of muscular dystrophy, or reductions in the expression of defective forms of dystrophin, such as the altered forms of dystrophin that are expressed in individuals with DMD or BMD. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include Duchenne muscular dystrophy (DMD) and Becker muscular dystrophy (BMD).

A “sugar” or “sugar moiety,” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′—S-sugars (e.g., 4′—S-ribose, 4′—S-2′-deoxyribose, and 4′—S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

“Treatment” and “treating,” as used herein, refer to the medical management of a subject with the intent to improve, ameliorate, or stabilize a disease, disorder, or condition (e.g., DMD or BMD). This term includes active treatment (treatment directed to improve DMD or BMD); palliative treatment (treatment designed for the relief of symptoms of DMD or BMD); and supportive treatment (treatment employed to supplement another therapy).

Throughout the description and claims of this specification, the words “comprise” and “contain” and variations of them mean “including but not limited to,” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references to “conjugates” also refer to solvates thereof, including pharmaceutically acceptable solvates thereof.

All references to “oligonucleotides” also refer to salts and/or solvates thereof, including pharmaceutically acceptable salts and/or solvates thereof.

Unless otherwise specified, all peptides are shown herein in N-terminus to C-terminus direction (left to right). Unless otherwise specified, all oligonucleotides are shown herein in 5′ to 3′ direction (left to right).

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

### BRIEF DESCRIPTION OF THE DRAWINGS

FIG. 1 is a plot demonstrating the dose response in key muscle groups in the conjugate-treated animals (n=2-4). TA is tibialis anterior. The graph is plotted as a mean±SD. One animal in the 40 mg/kg infusion group displayed lower levels in both the TA and heart reducing the group average.

FIGS. 2A and 2B are plots showing pre- and post-dosing (1, 4, and 7 days) serum urea and creatinine levels (kidney function markers) in animals (n=2-4). PD=pre-dose. The graphs are plotted as a mean±SD.

FIGS. 3A, 3B, and 3C are plots showing pre- and post-dosing (1, 4, and 7 days) serum alkaline phosphatase (ALP), alanine aminotransferase (ALT), and aspartate aminotransferase (AST) levels (liver function markers) in animals (n=2-4). PD=pre-dose. The graphs are plotted as a mean±SD.

FIGS. 4A, 4B, and 4C are plots showing pre- and post-dosing (1, 4, and 7 days) white blood cell counts, neutrophil blood counts, and monocyte blood counts (n=2-4). PD=pre-dose. The graphs are plotted as a mean±SD.

FIG. 5 is a plot showing post-dose plasma concentration of the PMO in animals treated with Conjugate 1 (n=2-4). EOI=end of infusion.

FIG. 6 is a plot summarizing the tissue bioanalysis results for the PMO concentration in animals treated with Conjugate 1 (n =2-4). The graph is plotted as a mean±SD. LLOQ=lower limit of linear quantification (50 ng/g).

FIG. 7 is a plot summarizing the tissue bioanalysis results for the PMO concentration in animals (n=2-4) receiving either 40 mg/kg bolus or 40 mg/kg infusion (30 minutes) of Conjugate 1. The graph is plotted as a mean±SD. LLOQ=lower limit of linear quantification (50 ng/g).

FIG. 8 is a plot summarizing the tissue bioanalysis results for the PMO concentration in animals (n=2-4) receiving either 60 mg/kg bolus or 60 mg/kg infusion (30 minutes) of Conjugate 1. The graph is plotted as a mean±SD. LLOQ=lower limit of linear quantification (50 ng/g).

FIGS. 9A and B are a plot summarizing the creatinine and KIM-1 urinalysis in the tested animals (n=2-4). The graph is plotted as a mean±SD.

FIGS. 10A, 10B, and 10C are plots showing coagulation marker levels (prothrombin time, activated partial thromboplastin time, and fibrinogen level) pre- and post-dosing (1 and 7 days) in animals dosed with Conjugate 1 (n=2-4). The graphs are plotted as a mean±SD.

FIG. 11 is a plot showing the body weights of test animals (n=2-4) during the study. The graph is plotted as a mean±SD.

FIGS. 12A-12I are plots showing the weights of the indicated tissues from animals infused with 0 mg/kg, 40 mg/kg, or 60 mg/kg Conjugate 1.

FIGS. 13A-E are plots showing no serum complement activation in CH₅₀, Bb, C3a, or SC5b-9. C5a was not detected during the study. The graphs are plotted as a mean±SD (n=2-4).

FIG. 14 is a series of plots showing exon 51 skipping by the indicated conjugates in biceps, quadriceps, and diaphragm.

FIG. 15 is a series of plots showing exon 51 skipping by the indicated conjugates in left ventricle, right ventricle, and aorta.

FIG. 16 is a plot showing creatinine kinase levels in mdx mice 1 week after administration of vehicle or conjugate 2 (60 mg/kg) and in wild-type (WT) mice 1 week after administration of vehicle.

FIG. 17A is a plot showing the exon skipping efficacy of Conjugate 2 and R6G—PMO in the quadriceps of mdx mice.

FIG. 17B is a plot showing dystrophin restoration efficacy of Conjugate 2 and R6G—PMO in the quadriceps of mdx mice. The results are normalized to WT mice.

FIG. 18A is a plot showing the exon skipping efficacy of Conjugate 2 and R6G—PMO in the mdx mice hearts.

FIG. 18B is a plot showing dystrophin restoration efficacy of Conjugate 2 and R6G—PMO in the mdx mice hearts. The results are normalized to WT mice.

FIG. 19A is a plot showing exon 51 skipping efficacy in biceps of nonhuman primates receiving a total of three doses of Conjugate 1 Q2W. Data shown as mean±SD; n=3 per group.

FIG. 19B is a plot showing exon 51 skipping efficacy in quadriceps of nonhuman primates receiving a total of three doses of Conjugate 1 Q2W. Data shown as mean±SD; n=3 per group.

### DETAILED DESCRIPTION

In general, the invention provides methods of treating a subject having Duchenne muscular dystrophy. The methods include administration of 1 mg/kg to 60 mg/kg of a conjugate of an oligonucleotide and a peptide covalently bonded or linked via a linker to the oligonucleotide to the subject (e.g., a subject amenable to exon 51 skipping).

In some embodiments, 30 mg/kg to 60 mg/kg (e.g., 40 mg/kg to 60 mg/kg; 30 mg/kg to 50 mg/kg; 30 mg/kg to 40 mg/kg; e.g., 30 mg/kg, 40 mg/kg, 50 mg/kg, or 60 mg/kg) of conjugate is administered.

In some embodiments, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, or 45 mg/kg to 60 mg/kg of conjugate is administered.

In some embodiments, 1 mg/kg to 30 mg/kg (e.g., 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, or 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of conjugate is administered e.g., 1 mg/kg, 4 mg/kg, 5 mg/kg, 6 mg/kg, 8 mg/kg, 10 mg/kg, 15 mg/kg, 20 mg/kg, 25 mg/kg, or 30 mg/kg) of conjugate is administered. In some embodiments, low doses (e.g., 1 mg/kg to 30 mg/kg) are administered to provide less drug exposure, and thus increase safety, while still maintaining sufficient efficacy.

In some embodiments, the conjugate is administered by infusion, e.g., intravenous infusion, which optionally is bolus infusion or continuous infusion, with that latter optionally being for 10 minutes to 3 hours, e.g., 0.25-2 hours, or 0.5-1 hour.

The peptide includes at least one cationic domain including at least 4 amino acid residues and at least one hydrophobic domain including at least 3 amino acid residues, provided that the peptide includes a total of 7 to 40 amino acid residues, and provided that the at least one cationic domain includes a beta-alanine residue in combination with arginine and/or histidine residues. The oligonucleotide including a total of 12 to 40 contiguous nucleobases, wherein at least 12 contiguous nucleobases are complementary to a target sequence in a human dystrophin gene.

Preferably, the conjugate comprises or consists of 5′-CTCCAACATCAAGGAAGATGGCATTTCTAG-3′ (SEQ ID NO: 106) having a 3′-terminus covalently linked via a glutamic acid residue to N-terminus of peptide RBRRBRFQILYBRBR—NH₂, wherein free —COOH, if any, in the glutamic acid residue is replaced with —CONH₂. For example, the 5′ group of this conjugate may be a phosphoramidate (e.g., sarcosinamide). Alternatively, the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYRBHBH, wherein free —COOH, if any, in the glutamic acid residue is replaced with —CONH₂. For example, the 5′ group of this conjugate may be a phosphoramidate. Alternatively, the conjugate comprises or consists of 10 5′-CTCCAACATCAAGGAAGATGGCATTTCTAG-3′ (SEQ ID NO: 106) having a 3′-terminus covalently linked via a beta-alanine residue to C-terminus of peptide Ac-RBRRBRFQILYRBHBH. For example, the 5′ group of this conjugate may be a phosphoramidate. Yet more preferably, the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYBRBR, wherein free —COOH, if any, in the glutamic acid residue is replaced with —CONH₂. For example, the 5′ group of this conjugate may be a phosphoramidate. In some embodiments, the conjugate including a glutamic acid residue linker has the structure:

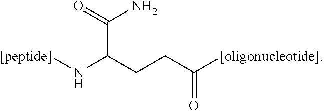

Advantageously, as described in the Examples section, the conjugate dosages described herein can induce skipping of dystrophin exon 51, even in heart tissues, of non-human primates without causing lasting liver and kidney toxicity. Advantageously, as described in the Examples section, the conjugate dosages described herein can deliver the cargo oligonucleotide even to traditionally hard to reach tissues. Use of the non-human primate model described herein advantageously has facilitated the identification of doses that can be used in other primates, such as humans.

Preferably, the conjugate is administered to the subject at a frequency that is weekly to monthly (e.g., weekly, biweekly, one every three weeks, or monthly) or quarterly.

### Oligonucleotides

Oligonucleotides used in the conjugates disclosed herein may be those complementary to a target site within the dystrophin (DMD) gene. Without wishing to be bound by theory, it is believed that an oligonucleotide hybridizing to certain target areas within a human dystrophin gene may induce the skipping of exon 51 during the dystrophin pre-mRNA splicing, thereby ameliorating Duchenne muscular dystrophy.

An oligonucleotide includes a nucleobase sequence complementary to a human dystrophin gene and, e.g., capable of inducing exon 51 skipping. In certain preferred embodiments, an oligonucleotide includes at least 12 (e.g., at least 15, at least 16, at least 17, at least 18, at least 19, or at least 20) contiguous nucleobases from 5′-CUCCAACAUCAAGGAAGAUGGCAUUUCUAG-3′ (SEQ ID NO: 112) or its thymine-substitution analogue, 5′-CTCCAACATCAAGGAAGATGGCATTTCTAG-3′ (SEQ ID NO: 106). In certain preferred embodiments, an oligonucleotide comprises or consists of 5′-CTCCAACATCAAGGAAGATGGCATTTCTAG-3′ (SEQ ID NO: 106). Non-limiting examples of oligonucleotides which may be used in the conjugates disclosed herein are those listed below.

| # SEQ | |
|---|---|
| ID | |
| NO: | Sequence |
| 107 | 5′-ACCAGAGUAACAGUCUGAGUAGGAGC-3′ |
| 108 | 5′-CUCAUACCUUCUGCUUGAUGAUC-3′ |
| 109 | 5′-UUCUGUCCAAGCCCGGUUGAAAUC-3′ |
| 110 | 5′-ACAUCAAGGAAGAUGGCAUUUCUAGUUUGG-3′ |
| 111 | 5′-ACAUCAAGGAAGAUGGCAUUUCUAG-3′ |
| 112 | 5′-CUCCAACAUCAAGGAAGAUGGCAUUUCUAG-3′ |
| 113 | 5′-AUCAUUUUUUCUCAUACCUUCUGCUAG-3′ |
| 114 | 5′-AUCAUUUUUUCUCAUACCUUCUGCUAGGAG |
| | CUAAAAAG-3′ |
| 115 | 5′-CACCCACCAUCACCCUCUGUG-3′ |
| 116 | 5′-AUCAUCUCGUUGAUAUCCUCAA-3′ |

In some embodiments, one or more uracils (e.g., all uracils) in an oligonucleotide sequence shown above are replaced with thymines. For example, an oligonucleotide sequence may be, e.g., 5′-CUCCAACAUCAAGGAAGAUGGCAUUUCUAG-3′ (SEQ ID NO: 112). Alternatively, the oligonucleotide sequence may be, e.g., 5′-CTCCAACATCAAGGAAGATGGCATTTCTAG-3′ (SEQ ID NO: 106).

In some embodiments of the above and other oligonucleotides described herein, the oligonucleotide is a phosphorodiamidate morpholino oligomer (PMO).

### Peptides

Peptides that may be used in the conjugates described herein include those disclosed in WO 2020030927 and WO 2020115494. Preferably, peptides included in the conjugates described herein include no artificial amino acid residues.

In some embodiments, the peptide does not contain aminohexanoic acid residues. In some embodiments, the peptide does not contain any form of aminohexanoic acid residues. In some embodiments, the peptide does not contain 6-aminohexanoic acid residues.

In some embodiments, the peptide contains only natural amino acid residues, and therefore consists of natural amino acid residues.

In some embodiments, artificial amino acids such as 6-aminohexanoic acid that are typically used in cell- penetrating peptides are replaced by natural amino acids. In some embodiments, the artificial amino acids such as 6-aminohexanoic acid that are typically used in cell-penetrating peptides are replaced by amino acids selected from beta-alanine, serine, proline, arginine and histidine or hydroxyproline.

In some embodiments, aminohexanoic acid is replaced by beta-alanine. In some embodiments, 6-aminohexanoic acid is replaced by beta-alanine

In some embodiments, aminohexanoic acid is replaced by histidine. In some embodiments, 6-aminohexanoic acid is replaced by histidine.

In some embodiments, aminohexanoic acid is replaced by hydroxyproline. In some embodiments, 6-aminohexanoic acid is replaced by hydroxyproline.

In some embodiments, the artificial amino acids such as 6-aminohexanoic acid that are typically used in cell-penetrating peptides may be replaced by a combination of any of beta-alanine, serine, proline, arginine and histidine or hydroxyproline, e.g., a combination of any of beta-alanine, histidine, and hydroxyproline.

In some embodiments, there is provided a peptide having a total length of 40 amino acid residues or less, the peptide comprising: two or more cationic domains each comprising at least 4 amino acid residues; and one or more hydrophobic domains each comprising at least 3 amino acid residues; wherein at least one cationic domain comprises histidine residues. In some embodiments, wherein at least one cationic domain is histidine rich.

In some embodiments, what is meant by histidine rich is defined herein in relation to the cationic domains.

### Cationic Domain

The present invention relates to short cell-penetrating peptides having a particular structure in which there are at least two cationic domains having a certain length.

In some embodiments, the peptide comprises up to 4 cationic domains, up to 3 cationic domains.

In some embodiments, the peptide comprises 2 cationic domains.

As defined above, the peptide comprises two or more cationic domains each having a length of at least 4 amino acid residues.

In some embodiments, each cationic domain has a length of between 4 to 12 amino acid residues, e.g., a length of between 4 to 7 amino acid residues.

In some embodiments, each cationic domain has a length of 4, 5, 6, or 7 amino acid residues.

In some embodiments, each cationic domain is of similar length, e.g., each cationic domain is the same length.

In some embodiments, each cationic domain comprises cationic amino acids and may also contain polar and or nonpolar amino acids.

Non-polar amino acids may be selected from: alanine, beta-alanine, proline, glycine, cysteine, valine, leucine, isoleucine, methionine, tryptophan, phenylalanine. In some embodiments, non-polar amino acids do not have a charge.

Polar amino acids may be selected from: serine, asparagine, hydroxyproline, histidine, arginine, threonine, tyrosine, and glutamine. In some embodiments, the selected polar amino acids do not have a negative charge.

Cationic amino acids may be selected from: arginine, histidine, and lysine. In some embodiments, cationic amino acids have a positive charge at physiological pH.

In some embodiments, each cationic domain does not comprise anionic or negatively charged amino acid residues. In some embodiments, each cationic domain comprises arginine, histidine, beta-alanine, hydroxyproline, and/or serine residues.

In some embodiments, each cationic domain comprises or consists of arginine, histidine, beta-alanine, hydroxyproline, and/or serine residues.

In some embodiments, each cationic domain comprises at least 40%, at least 45%, or at least 50% cationic amino acids.

In some embodiments, each cationic domain comprises a majority of cationic amino acids. In some embodiments, each cationic domain comprises at least 55%, at least 60%, at least 65% at least 70%, at least 75%, at least 80%, at least 85%, at least 90%, or at least 95% cationic amino acids.

In some embodiments, each cationic domain comprises an isoelectric point (pi) of at least 7.5, at least 8.0, at least 8.5, at least 9.0, at least 9.5, at least 10.0, at least 10.5, at least 11.0, at least 11.5, or at least 12.0.

In some embodiments, each cationic domain comprises an isoelectric point (pi) of at least 10.0.

In some embodiments, each cationic domain comprises an isoelectric point (pi) of between 10.0 and 13.0

In some embodiments, each cationic domain comprises an isoelectric point (pi) of between 10.4 and 12.5.

In some embodiments, the isoelectric point of a cationic domain is calculated at physiological pH by any suitable means available in the art. In some embodiments, by using the I PC (www.isoelectric.org) a web-based algorithm developed by Lukasz Kozlowski, Biol Direct. 2016; 11:55. DOI: 10.1186/s 13062-016-0159-9.

In some embodiments, each cationic domain comprises at least 1 cationic amino acid, e.g., 1-5 cationic amino acids. In some embodiments, each cationic domain comprises at least 2 cationic amino acids, e.g., 2-5 cationic amino acids.

In some embodiments, each cationic domain is arginine rich and/or histidine rich. In some embodiments, a cationic domain may contain both histidine and arginine.

In some embodiments, each cationic domain comprises a majority of arginine and/or histidine residues.

In some embodiments, each cationic domain comprises at least 40%, at least 45%, at least 50%, at least 55%, at least 60%, at least 60%, at least 65%, or at least 70% arginine and/or histidine residues.

In some embodiments, a cationic domain may comprise at least 40%, at least 45%, at least 50%, at least 55%, at least 60%, at least 60%, at least 65%, or at least 70% arginine residues.

In some embodiments, a cationic domain may comprise at least 40%, at least 45%, at least 50%, at least 55%, at least 60%, at least 60%, at least 65%, or at least 70% histidine residues.

In some embodiments, a cationic domain may comprise a total of between 1-5 histidine and 1-5 arginine residues. In some embodiments, a cationic domain may comprise between 1-5 arginine residues. In some embodiments, a cationic domain may comprise between 1-5 histidine residues. In some embodiments, a cationic domain may comprise a total of between 2-5 histidine and 3-5 arginine residues. In some embodiments, a cationic domain may comprise between 3-5 arginine residues. In some embodiments, a cationic domain may comprise between 2-5 histidine residues.

In some embodiments, each cationic domain comprises one or more beta-alanine residues. In some embodiments, each cationic domain may comprise a total of between 2-5 beta-alanine residues, e.g., a total of 2 or 3 beta-alanine residues.

In some embodiments, a cationic domain may comprise one or more hydroxyproline residues or serine residues.

In some embodiments, a cationic domain may comprise between 1-2 hydroxyproline residues. In some embodiments, a cationic domain may comprise between 1-2 serine residues.

In some embodiments, all of the cationic amino acids in a given cationic domain may be histidine, alternatively, e.g., all of the cationic amino acids in a given cationic domain may be arginine.

In some embodiments, the peptide may comprise at least one histidine rich cationic domain. In some embodiments, the peptide may comprise at least one arginine rich cationic domain.

In some embodiments, the peptide may comprise at least one arginine rich cationic domain and at least one histidine rich cationic domain.

In some embodiments, the peptide comprises two arginine rich cationic domains.

In some embodiments, the peptide comprises two histidine rich cationic domains.

In some embodiments, the peptide comprises two arginine and histidine rich cationic domains.

In some embodiments, the peptide comprises one arginine rich cationic domain and one histidine rich cationic domain. In some embodiments, each cationic domain comprises no more than 3 contiguous arginine residues, e.g., no more than 2 contiguous arginine residues.

In some embodiments, each cationic domain comprises no contiguous histidine residues.

In some embodiments, each cationic domain comprises arginine, histidine and/or beta-alanine residues. In some embodiments, each cationic domain comprises a majority of arginine, histidine and/or beta-alanine residues. In some embodiments, at least 70%, at least 75%, at least 80%, at least 85%, at least 90%, at least 95%, or 100% of the amino acid residues in each cationic domain are arginine, histidine and/or beta-alanine residues. In some embodiments, each cationic domain comprises or consists of arginine, histidine and/or beta-alanine residues.

In some embodiments, the peptide comprises a first cationic domain comprising arginine and beta-alanine residues and a second cationic domain comprising arginine and beta-alanine residues.

In some embodiments, the peptide comprises a first cationic domain comprising arginine and beta-alanine resides, and a second cationic domain comprising histidine, beta-alanine, and optionally arginine residues.

In some embodiments, the peptide comprises a first cationic domain comprising arginine and beta-alanine resides, and a second cationic domain comprising histidine and beta-alanine residues.

In some embodiments, the peptide comprises a first cationic domain consisting of arginine and beta-alanine residues and a second cationic domain consisting of arginine and beta-alanine residues.

In some embodiments, the peptide comprises a first cationic domain consisting of arginine and beta-alanine residues and a second cationic domain consisting of arginine, histidine and beta- alanine residues.

In some embodiments, the peptide comprises at least two cationic domains, e.g., these cationic domains form the arms of the peptide. In some embodiments, the cationic domains are located at the N and C terminus of the peptide. In some embodiments, therefore, the cationic domains may be known as the cationic arm domains.

In some embodiments, the peptide comprises two cationic domains, wherein one is located at the N-terminus of the peptide and one is located at the C-terminus of the peptide. In some embodiments, at either end of the peptide. In some embodiments, no further amino acids or domains are present at the N-terminus and C-terminus of the peptide, with the exception of other groups such as a terminal modification, linker and/or oligonucleotide. For the avoidance of doubt, such other groups may be present in addition to ‘the peptide’ described and claimed herein. In some embodiments, therefore each cationic domain forms the terminus of the peptide. In some embodiments, this does not preclude the presence of a further linker group as described herein.

In some embodiments, the peptide may comprise up to 4 cationic domains. In some embodiments, the peptide comprises two cationic domains.

In some embodiments, the peptide comprises two cationic domains that are both arginine rich.

In some embodiments, the peptide comprises one cationic domain that is arginine rich.

In some embodiments, the peptide comprises two cationic domains that are both arginine and histidine rich.

In some embodiments, the peptide comprises one cationic domain that is arginine rich and one cationic domain that is histidine rich.

In some embodiments, the cationic domains comprise amino acid units selected from the following: R, H, B, RR, HH, BB, RH, HR, RB, BR, HB, BH, RBR, RBB, BRR, BBR, BRB, RBH, RHB, HRB, BRH, HRR, RRH, HRH, HBB, BBH, RHR, BHB, HBH, or any combination thereof.

In some embodiments, a cationic domain may also include serine, proline and/or hydroxyproline residues. In some embodiments, the cationic domains may further comprise amino acid units selected from the following: RP, PR, RPR, RRP, PRR, PRP, Hyp; R[Hyp]R, RR[Hyp], [Hyp]RR, [Hyp]R[Hyp], [Hyp][Hyp]R, R[Hyp][Hyp], SB, BS, or any combination thereof, or any combination with the above listed amino acid units.

In some embodiments, each cationic domain comprises any one of the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19), or any combination thereof.

In some embodiments, each cationic domain comprises or consists of any one of the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B, R[Hyp]H[Hyp]HB, R[Hyp]RR[Hyp]R (SEQ ID NO: 19), or any combination thereof.

In some embodiments, each cationic domain comprises or consists of one of the following sequences: RBRRBRR (SEQ ID NO: 1), RBRBR (SEQ ID NO: 2), RBRRBR (SEQ ID NO: 4), BRBR (SEQ ID NO: 7), RBHBH (SEQ ID NO: 8), or HBHBR (SEQ ID NO: 9).

In some embodiments, each cationic domain in the peptide may be identical or different. In some embodiments, each cationic domain in the peptide is different.

### Hydrophobic Domain

The present invention relates to short cell-penetrating peptides having a particular structure in which there is at least one hydrophobic domain having a certain length.

References to ‘hydrophobic’ herein denote an amino acid or domain of amino acids having the ability to repel water or which do not mix with water.

In some embodiments, the peptide comprises up to 3 hydrophobic domains or up to 2 hydrophobic domains. In some embodiments, the peptide comprises 1 hydrophobic domain.

As defined above, the peptide comprises one or more hydrophobic domains each having a length of at least 3 amino acid residues.

In some embodiments, each hydrophobic domain has a length of between 3-6 amino acids. In some embodiments, each hydrophobic domain has a length of 5 amino acids.

In some embodiments, each hydrophobic domain may comprise nonpolar, polar, and hydrophobic amino acid residues.

Hydrophobic amino acid residues may be selected from: alanine, valine, leucine, isoleucine, phenylalanine, tyrosine, methionine, and tryptophan.

Non-polar amino acid residues may be selected from: proline, glycine, cysteine, alanine, valine, leucine, isoleucine, tryptophan, phenylalanine, and methionine.

Polar amino acid residues may be selected from: serine, asparagine, hydroxyproline, histidine, arginine, threonine, tyrosine, and glutamine.

In some embodiments, the hydrophobic domains do not comprise hydrophilic amino acid residues.

In some embodiments, each hydrophobic domain comprises a majority of hydrophobic amino acid residues. In some embodiments, each hydrophobic domain comprises at least 70%, at least 75%, at least 80%, at least 85%, at least 90%, at least 95%, or 100% hydrophobic amino acids. In some embodiments, each hydrophobic domain consists of hydrophobic amino acid residues.

In some embodiments, each hydrophobic domain comprises a hydrophobicity of at least 0.3, at least 0.4, at least 0.5, at least 0.6, at least 0.7, at least 0.8, at least 0.8, at least 1.0, at least 1.1, at least 1.2, or at least 1.3.

In some embodiments, each hydrophobic domain comprises a hydrophobicity of at least 0.3, at least 0.35, at least 0.4, or at least 0.45.

In some embodiments, each hydrophobic domain comprises a hydrophobicity of at least 1.2, at least 1.25, at least 1.3, or at least 1.35.

In some embodiments, each hydrophobic domain comprises a hydrophobicity of between 0.4 and 1.4

In some embodiments, each hydrophobic domain comprises of a hydrophobicity of between 0.45 and 0.48.

In some embodiments, each hydrophobic domain comprises a hydrophobicity of between 1.27 and 1.39

In some embodiments, hydrophobicity is as measured by White and Wimley: W. C. Wimley and S. H. White, “Experimentally determined hydrophobicity scale for proteins at membrane interfaces” Nature Struct Biol 3:842 (1996).

In some embodiments, each hydrophobic domain comprises at least 3 or at least 4 hydrophobic amino acid residues.

In some embodiments, each hydrophobic domain comprises phenylalanine, leucine, Isoleucine, tyrosine, tryptophan, proline, and/or glutamine residues. In some embodiments, each hydrophobic domain comprises or consists of phenylalanine, leucine, isoleucine, tyrosine, tryptophan, proline, and/or glutamine residues.

In some embodiments, each hydrophobic domain comprises or consists of phenylalanine, leucine, isoleucine, tyrosine and/or glutamine residues.

In some embodiments, each hydrophobic domain comprises or consists of tryptophan and/or proline residues.

In some embodiments, the peptide comprises one hydrophobic domain. In some embodiments, the or each hydrophobic domain is located in the center of the peptide. In some embodiments, therefore, the hydrophobic domain may be known as a core hydrophobic domain. In some embodiments, the or each hydrophobic core domain is flanked on either side by an arm domain. In some embodiments, the arm domains may comprise one or more cationic domains and one or more further hydrophobic domains.

In some embodiments, each arm domain comprises a cationic domain.

In some embodiments, the peptide comprises two arm domains flanking a hydrophobic core domain, wherein each arm domain comprises a cationic domain.

In some embodiments, the peptide comprises or consists of two cationic arm domains flanking a hydrophobic core domain.

In some embodiments, the or each hydrophobic domain comprises one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26), or any combination thereof.

In some embodiments, the or each hydrophobic domain comprises or consists of one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26), or any combination thereof.

In some embodiments, the or each hydrophobic domain comprises or consists of one of the following sequences FQILY (SEQ ID NO: 21), YQFLI (SEQ ID NO: 20), or ILFQY (SEQ ID NO: 22).

In some embodiments, the or each hydrophobic domain comprises or consists of FQILY (SEQ ID NO: 21).

In some embodiments, each hydrophobic domain in the peptide may have the same sequence or a different sequence.

The present invention relates to short cell-penetrating peptides for use in transporting therapeutic cargo molecules in the treatment of medical conditions.

The peptide has a sequence that is a contiguous single molecule, therefore the domains of the peptide are contiguous. In some embodiments, the peptide comprises several domains in a linear arrangement between the N-terminus and the C-terminus. In some embodiments, the domains are selected from cationic domains and hydrophobic domains described above. In some embodiments, the peptide comprises or consists of cationic domains and hydrophobic domains wherein the domains are as defined above.

Each domain has common sequence characteristics as described in the relevant sections above, but the exact sequence of each domain is capable of variation and modification. Thus, a range of sequences is possible for each domain. The combination of each possible domain sequence yields a range of peptide structures, each of which form part of the present invention. Features of the peptide structures are described below.

In some embodiments, a hydrophobic domain separates any two cationic domains. In some embodiments, each hydrophobic domain is flanked by cationic domains on either side thereof.

In some embodiments, no cationic domain is contiguous with another cationic domain.

In some embodiments, the peptide comprises one hydrophobic domain flanked by two cationic domains in the following arrangement:

[cationic domain]-[hydrophobic domain]-[cationic domain]

In some embodiments, the hydrophobic domain may be known as the core domain and each of the cationic domains may be known as an arm domain. In some embodiments, the hydrophobic arm domains flank the cationic core domain on either side thereof.

In some embodiments, the peptide comprises or consists of two cationic domains and one hydrophobic domain.

In some embodiments, the peptide comprises or consists of one hydrophobic core domain flanked by two cationic arm domains.

In some embodiments, the peptide comprises or consists of one hydrophobic core domain comprising a sequence selected from: YQFLI (SEQ ID NO: 20), FQILY (SEQ ID NO: 21), ILFQY (SEQ ID NO: 22), FQIY (SEQ ID NO: 23), WWW, WWVPWW (SEQ ID NO: 24), WPWW (SEQ ID NO: 25), and WWPW (SEQ ID NO: 26), flanked by two cationic arm domains each comprising a sequence selected from: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19).

In some embodiments, the peptide comprises or consists of one hydrophobic core domain comprising a sequence selected from: FQILY (SEQ ID NO: 21), YQFLI (SEQ ID NO: 20), and ILFQY (SEQ ID NO: 22), flanked by two cationic arm domains comprising a sequence selected from: RBRRBRR (SEQ ID NO: 1), RBRBR (SEQ ID NO: 2), RBRRBR (SEQ ID NO: 4), BRBR (SEQ ID NO: 7), RBHBH (SEQ ID NO: 8), and HBHBR (SEQ ID NO: 9). In some embodiments, the peptide comprises or consists of one hydrophobic core domain comprising the sequence: FQILY (SEQ ID NO: 21), flanked by two cationic arm domains comprising a sequence selected from: RBRRBRR (SEQ ID NO: 1), RBRBR (SEQ ID NO: 2), RBRRBR (SEQ ID NO: 4), BRBR (SEQ ID NO: 7), and RBHBH (SEQ ID NO: 8).

In any such embodiment, further groups may be present such as a linker, terminal modification and/or oligonucleotide.

In some embodiments, the peptide is N-terminally modified.

In some embodiments, the peptide is N-acetylated, N-methylated, N-trifluoroacetylated, N-trifluoromethylsulfonylated, or N-methylsulfonylated. In some embodiments, the peptide is N-acetylated.

Optionally, the N-terminus of the peptide may be unmodified.

In some embodiments, the peptide is N-acetylated.

In some embodiments, the peptide is C-terminal modified.

In some embodiments, the peptide comprises a C-terminal modification selected from: carboxy-, thioacid-, aminooxy-, hydrazino-, thioester-, azide, strained alkyne, strained alkene, aldehyde-, thiol or haloacetyl-group.

Advantageously, the C-terminal modification provides a means for linkage of the peptide to the oligonucleotide.

Accordingly, the C-terminal modification may comprise the linker and vice versa. In some embodiments, the C-terminal modification may consist of the linker or vice versa. Suitable linkers are described herein elsewhere.

In some embodiments, the peptide comprises a C-terminal carboxyl group.

In some embodiments, the C-terminal carboxyl group is provided by a glycine or beta-alanine residue.

In some embodiments, the C terminal carboxyl group is provided by a beta-alanine residue. In some embodiments, the C terminal beta-alanine residue is a linker.

In some embodiments, therefore each cationic domain may further comprise an N or C terminal modification. In some embodiments, the cationic domain at the C terminus comprises a C-terminal modification. In some embodiments, the cationic domain at the N terminus comprises a N-terminal modification. In some embodiments, the cationic domain at the C terminus comprises a linker group, In some embodiments, the cationic domain at the C terminus comprises a C-terminal beta-alanine. In some embodiments, the cationic domain at the N terminus is N-acetylated.

The peptide of the present invention is defined as having a total length of 40 amino acid residues or less. The peptide may therefore be regarded as an oligopeptide.

In some embodiments, the peptide has a total length of 3-30 amino acid residues, e.g., of 5-25 amino acid residues, of 10-25 amino acid residues, of 13-23 amino acid residues, or of 15-20 amino acid residues.

In some embodiments, the peptide has a total length of at least 12, at least 13, at least 14, at least 15, at least 16, or at least 17 amino acid residues.

In some embodiments, the peptide is capable of penetrating cells. The peptide may therefore be regarded as a cell-penetrating peptide.

In some embodiments, the peptide is for attachment to an oligonucleotide. In some embodiments, the peptide is for transporting an oligonucleotide into a target cell. In some embodiments, the peptide is for delivering an oligonucleotide into a target cell. The peptide may therefore be regarded as a carrier peptide.

In some embodiments, the peptide is capable of penetrating into cells and tissues, e.g., into the nucleus of cells. In some embodiments, into muscle tissues.

In some embodiments, the peptide may comprise or consist of a peptide selected from any one of the following sequences:

(SEQ ID NO: 27)
RBRRBRRFQILYRBRBR
(SEQ ID NO: 28)
RBRRBRRFQILYRBRR
(SEQ ID NO: 29)
RBRRBRFQILYRRBRBR
(SEQ ID NO: 30)
RBRBRFQILYRBRRBRR
(SEQ ID NO: 31)
RBRRBRRYQFLIRBRBR
(SEQ ID NO: 32)
RBRRBRRILFQYRBRBR
(SEQ ID NO: 33)
RBRRBRFQILYRBRBR
(SEQ ID NO: 34)
RBRRBFQILYRBRRBR
(SEQ ID NO: 35)
RBRRBRFQILYBRBR
(SEQ ID NO: 36)
RBRRBFQILYRBRBR
(SEQ ID NO: 37)
RBRRBRRFQILYRBHBH
(SEQ ID NO: 38)
RBRRBRRFQILYHBHBR
(SEQ ID NO: 39)
RBRRBRRFQILYHBRBH
(SEQ ID NO: 40)
RBRRBRRYQFLIRBHBH
(SEQ ID NO: 41)
RBRRBRRILFQYRBHBH
(SEQ ID NO: 42)
RBRHBHRFQILYRBRBR
(SEQ ID NO: 43)
RBRBBHRFQILYRBHBH
(SEQ ID NO: 44)
RBRRBRFQILYRBHBH
(SEQ ID NO: 45)
RBRRBRFQILYHBHBH
(SEQ ID NO: 46)
RBRRBHFQILYRBHBH
(SEQ ID NO: 47)
HBRRBRFQILYRBHBH
(SEQ ID NO: 48)
RBRRBFQILYRBHBH
(SEQ ID NO: 49)
RBRRBRFQILYBHBH
(SEQ ID NO: 50)
RBRRBRYQFLIHBHBH
(SEQ ID NO: 51)
RBRRBRILFQYHBHBH
(SEQ ID NO: 52)
RBRRBRRFQILYHBHBH

In some embodiments, the peptide may comprise or consist of a peptide selected from any one of the following additional sequences:

(SEQ ID NO: 53)
RBRRBRFQILYBRBS
(SEQ ID NO: 54)
RBRRBRFQILYBRB[Hyp]
(SEQ ID NO: 55)
RBRRBRFQILYBR[Hyp]R
(SEQ ID NO: 56)
RRBRRBRFQILYBRBR
(SEQ ID NO: 57)
BRRBRRFQILYBRBR
(SEQ ID NO: 58)
RBRRBRWWWBRBR
(SEQ ID NO: 59)
RBRRBRWWPWWBRBR
(SEQ ID NQ:60)
RBRRBRWPWWBRBR
(SEQ ID NO: 61)
RBRRBRWWPWBRBR
(SEQ ID NO: 62)
RBRRBRRWWWRBRBR
(SEQ ID NO: 63)
RBRRBRRWWPWWRBRBR
(SEQ ID NO: 64)
RBRRBRRWPWWRBRBR
(SEQ ID NO: 65)
RBRRBRRWWPWRBRBR
(SEQ ID NO: 66)
RBRRBRRFQILYBRBR
(SEQ ID NO: 67)
RBRRBRRFQILYRBR
(SEQ ID NO: 68)
BRBRBWWPWWRBRRBR
(SEQ ID NO: 69)
RBRRBRRFQILYBHBH
(SEQ ID NO: 70)
RBRRBRRFQIYRBHBH
(SEQ ID NO: 71)
RBRRBRFQILYBRBH
(SEQ ID NO: 72)
RBRRBRFQILYR[Hyp]H[Hyp]H
(SEQ ID NO: 73)
R[Hyp]RR[Hyp]RFQILYRBHBH
(SEQ ID NO: 74)
R[Hyp]RR[Hyp]RFQILYR[Hyp]H[Hyp]H
(SEQ ID NO: 75)
RBRRBRWWWRBHBH
(SEQ ID NO: 76)
RBRRBRWWPRBHBH
(SEQ ID NO: 77)
RBRRBRPWWRBHBH
(SEQ ID NO: 78)
RBRRBRWWPWWRBHBH
(SEQ ID NO: 79)
RBRRBRWWPWRBHBH
(SEQ ID NO: 80)
RBRRBRWPWWRBHBH
(SEQ ID NO: 81)
RBRRBRRWWWRBHBH
(SEQ ID NO: 82)
RBRRBRRWWPWWRBHBH
(SEQ ID NO: 83)
RBRRBRRWPWWRBHBH
(SEQ ID NO: 84)
RBRRBRRWWPWRBHBH
(SEQ ID NO: 85)
RRBRRBRFQILYRBHBH
(SEQ ID NO: 86)
BRRBRRFQILYRBHBH
(SEQ ID NO: 87)
RRBRRBRFQILYBHBH
(SEQ ID NO: 88)
BRRBRRFQILYBHBH
(SEQ ID NO: 89)
RBRRBHRFQILYRBHBH
(SEQ ID NO: 101)
RBRRBRFQILY[Hyp]R[Hyp]R
(SEQ ID NO: 102)
R[Hyp]RR[Hyp]RFQILYBRBR
(SEQ ID NO: 103)
R[Hyp]RR[Hyp]RFQILY[Hyp]R[Hyp]R
(SEQ ID NO: 104)
RBRRBRWWWBRBR
(SEQ ID NO: 105)
RBRRBRWWPWWBRBR

In some embodiments, the peptide may comprise or consist of a peptide selected from one of the following sequences:

(SEQ ID NO: 27)
RBRRBRRFQILYRBRBR
(SEQ ID NO: 31)
RBRRBRRYQFLIRBRBR
(SEQ ID NO: 32)
RBRRBRRILFQYRBRBR
(SEQ ID NO: 35)
RBRRBRFQILYBRBR
(SEQ ID NO: 37)
RBRRBRRFQILYRBHBH
(SEQ ID NO: 38)
RBRRBRRFQILYHBHBR
(SEQ ID NO: 44)
RBRRBRFQILYRBHBH

In some embodiments, the peptide comprises or consists of the following sequence:

(SEQ ID NO: 35)
RBRRBRFQILYBRBR.

In some embodiments, the peptide comprises or consists of the following sequence:

(SEQ ID NO: 37)
RBRRBRRFQILYRBHBH.

In some embodiments, the peptide comprises or consists of the following sequence:

(SEQ ID NO: 44)
RBRRBRFQILYRBHBH.

### Conjugate

In some embodiments, the conjugate comprises a peptide selected from one of the following sequences: RBRRBRFQILYBRBR (SEQ ID NO: 35), RBRRBRRFQILYRBHBH (SEQ ID NO: 37), and RBRRBRFQILYRBHBH (SEQ ID NO: 44).

In some embodiments, in any case, the peptide may further comprise N-terminal modifications as described above.

Suitable linkers include, for example, a C-terminal cysteine residue that permits formation of a disulfide, thioether or thiol-maleimide linkage, a C-terminal aldehyde to form an oxime, a click reaction or formation of a morpholino linkage with a basic amino acid on the peptide or a carboxylic acid moiety on the peptide covalently conjugated to an amino group to form a carboxamide linkage.

In some embodiments, the linker is between 1-5 amino acids in length. In some embodiments, the linker may comprise any linker that is known in the art. In some embodiments, the linker is selected from any of the following sequences: G, BC, XC, C, GGC, BBC, BXC, XBC, X, XX, B, BB, BX and XB. In some embodiments, wherein X is 6-aminohexanoic acid. In some embodiments the linker is a Glu linker.

In some embodiments, the linker may be a polymer, such as for example PEG.

In some embodiments, the linker is beta-alanine.

In some embodiments, the peptide is conjugated to the oligonucleotide through a carboxamide linkage.

The linker of the conjugate may form part of the oligonucleotide to which the peptide is attached. Alternatively, the attachment of the oligonucleotide may be directly linked to the C-terminus of the peptide. In some embodiments, in such embodiments, no linker is required.

Alternatively, the peptide may be chemically conjugated to the oligonucleotide. Chemical linkage may be via a disulfide, alkenyl, alkynyl, aryl, ether, thioether, triazole, amide, carboxamide, urea, thiourea, semicarbazide, carbazide, hydrazine, oxime, phosphate, phosphoramidate, thiophosphate, boranophosphate, iminophosphates, or thiol-maleimide linkage, for example.

Optionally, cysteine may be added at the N-terminus of a peptide to allow for disulfide bond formation to the peptide, or the N-terminus may undergo bromoacetylation for thioether conjugation to the peptide.

In some embodiments, the conjugate is capable of penetrating into cells and tissues, e.g., into the nucleus of cells, e.g., into muscle tissues.

In some embodiments, the oligonucleotide component of the conjugate is as described in the “Oligonucleotide” section, above, and elsewhere herein. In some embodiments, the oligonucleotide is a phosphorodiamidate morpholino oligomer (PMO).

### Linkers

In addition to the above, conjugates described herein may include a linker covalently linking a peptide described herein to an oligonucleotide described herein. Linkers useful in the present invention can be found in WO 2020/115494, the disclosure of which is incorporated herein by reference.

The linker may be of formula (I):

T₁—(CR¹R²)ₙ—T₂. (I)

where

T₁ is a divalent group for attachment to the peptide and is selected from the group consisting of —NH— and carbonyl;

T₂ is a divalent group for attachment to an oligonucleotide and is selected from the group consisting of —NH— and carbonyl;

n is 1 , 2 or 3;

each R¹ is independently —Y¹ —X¹ —Z¹,

where
Y¹ is absent or —(CR^{A1}R^{A2})ₘ—, where m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —N(R^{A3})C(N R^{A3})N(R^{A3})—, —SO—, —S—, —SO₂—, —S(O)₂N(R^{A3})—, or —N(R^{A3})SO₂—, where each R^{A3} is independently selected from hydrogen and methyl; and

Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl,

where each (1-6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4} R^{A5}, and (1-4C)alkoxy, where R^{A4} and R^{A5} are each independently selected from the group consisting of hydrogen and (1-4C)alkyl; and

each R² is independently —Y²—X²—Z², where
Y² is absent or a group of the formula —[CR^{B1} R^{B2}]ₙ— in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})—, or —N(R^{B3})—SO₂—, where each R^{B3} is independently selected from hydrogen or methyl; and
Z² is selected from hydrogen, (1-6C)alkyl, (2-6C)alkenyl, (2-6C)alkynyl, aryl, (3-6C)cycloalkyl, (3-6C)cycloalkenyl or heteroaryl, where each (1-6C)alkyl, (2- 6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4} R^{B5}, and (1-4C)alkoxy, where R^{B4} and R^{B5} are each independently hydrogen or (1-2C)alkyl; with the proviso that; when n=1 and T₁ and T₂ are different to one another, then R¹ and R² are not both H; when n=1, T₁ and T₂ are different to one another and one of R¹ and R² is H then the other of R¹ and R² is not methyl; or when n=2 and each occurrence of R¹ and R² is H, then T₁ and T₂ are both —C(O)— or are both —NH—.

In some embodiments, the linker is of the following structure:

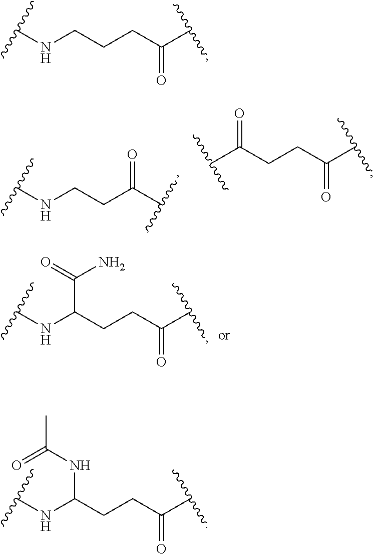

### Pharmaceutical Compositions

The conjugate of the invention, or a pharmaceutically acceptable salt thereof, may formulated into a pharmaceutical composition.

In some embodiments, the pharmaceutical composition comprises a conjugate of the invention or a pharmaceutically acceptable salt thereof.

In some embodiments, the pharmaceutical composition may further comprise a pharmaceutically acceptable diluent, adjuvant, or carrier.

Suitable pharmaceutically acceptable diluents, adjuvants and carriers are well known in the art.

It should be understood that the pharmaceutical compositions of the present disclosure can further include additional known therapeutic agents, drugs, modifications of compounds into prodrugs, and the like for alleviating, mediating, preventing, and treating the diseases, disorders, and conditions described herein under medical use.

In some embodiments, the pharmaceutical composition is for use as a medicament, e.g., for use as a medicament in the same manner as described herein for the conjugate. All features described herein in relation to medical treatment using the conjugate apply to the pharmaceutical composition.

Accordingly, in a further aspect of the invention there is provided a pharmaceutical composition according to the fourth aspect for use as a medicament. In a further aspect, there is provided a method of treating a subject for a disease condition comprising administering an effective amount of a pharmaceutical composition disclosed herein.

### Medical use

The conjugate comprising the peptide of the invention may be used as a medicament for the treatment of a disease using the dosages described above.

The medicament may be in the form of a pharmaceutical composition as defined above.

A method of treatment of a patient or subject in need of treatment for a disease condition is also provided, the method comprising the step of administering a therapeutically effective amount of the conjugate to the patient or subject. In some embodiments, the medical treatment requires delivery of the oligonucleotide into a cell, e.g., into the nucleus of the cell.

Diseases to be treated may include any disease where improved penetration of the cell and/or nuclear membrane by an oligonucleotide may lead to an improved therapeutic effect.

In some embodiments, the conjugate is for use in the treatment of diseases of the neuromuscular system.

In some embodiments, the conjugate is for use in the treatment of diseases caused by splicing deficiencies. In such embodiments, the oligonucleotide may comprise an oligonucleotide capable of preventing or correcting the splicing defect and/or increasing the production of correctly spliced mRNA molecules.

In some embodiments, the conjugate is for use in the treatment of DMD or BMD.

In some embodiments, the conjugate is for use in treating cardiac effects of a disease such as DMD including, e.g., cardiomyopathy (e.g., dilated, hypertrophic, or restrictive cardiomyopathy), heart failure, and/or cardiac arrhythmias.

In some embodiments, in such an embodiment, the oligonucleotide of the conjugate is operable to increase expression of the dystrophin protein. In some embodiments, in such an embodiment, the oligonucleotide of the conjugate is operable to increase the expression of functional dystrophin protein.

In some embodiments, the conjugate increases dystrophin expression by 10%, 15%, 20%, 25%, 30%, 35%, 40%, 45%, 50%, 55%, 60%, 65%, 70%. In some embodiments, the conjugate increases dystrophin expression by up to 50%. In some embodiments, the conjugate restores dystrophin protein expression by 10%, 15%, 20%, 25%, 30%, 35%, 40%, 45%, 50%, 55%, 60%, 65%, 70%. In some embodiments, the conjugate restores dystrophin protein expression by up to 50%.

In some embodiments, the conjugate restores dystrophin protein function by 10%, 15%, 20%, 25%, 30%, 35%, 40%, 45%, 50%, 55%, 60%, 65%, 70%. In some embodiments, the conjugate restores dystrophin protein function by up to 50%.

In some embodiments, the oligonucleotide of the conjugate is operable to do so by causing skipping of exon 51 during dystrophin transcription.

In some embodiments, the oligonucleotide of the conjugate causes 10%, 15%, 20%, 25%, 30%, 35%, 40%, 45%, 50%, 55%, 60%, 65%, 70%, 75%, 80%, 85% skipping of one or more exons of the dystrophin gene. In some embodiments, the oligonucleotide of the conjugate causes up to 50% skipping of one or more exons of the dystrophin gene.

In some embodiments, the patient or subject to be treated may be any animal or human. In some embodiments, the patient or subject may be a non-human mammal. In some embodiments, the patient or subject may be male or female. In some embodiments, the subject is male.

In some embodiments, the patient or subject to be treated may be any age. In some embodiments, the patient or subject to be treated is aged between 0-40 years, e.g., 0-30, e.g., 0-25, e.g., 0-20 years of age.

In some embodiments, the conjugate is for administration to a subject systemically for example by intramedullary, intrathecal, intraventricular, intravitreal, enteral, parenteral, intravenous, intra-arterial, intramuscular, intratumoral, subcutaneous oral or nasal routes.

In some embodiments, the conjugate is for administration to a subject intravenously.

In some embodiments, the conjugate is for administration to a subject intravenously by injection.

In some embodiments, the conjugate is administered by intravenous infusion. In some embodiments, the intravenous infusion is a bolus infusion. In some embodiments the intravenous is continuous for 10minutes-3 hours, e.g., 0.25- 2 hours or 0.5-1 hour.

Advantageously, the dosage of the conjugates of the present invention may be lower, e.g., an order or magnitude lower, than the dosage required to see any effect from the oligonucleotide alone.

In some embodiments, after administration of the conjugates of the present invention, one or more markers of toxicity are significantly reduced compared to prior conjugates using currently available peptide carriers

Suitable markers of toxicity may be markers of nephrotoxicity.

Suitable urine and serum markers of toxicity include KIM-1, NGAL, BUN, creatinine, alkaline phosphatase, alanine transferase, and aspartate aminotransferase.

In some embodiments, the level of at least one of KIM-1, NGAL, and BUN is reduced after administration of the conjugates of the present invention when compared to prior conjugates using currently available peptide carriers.

In some embodiments, the levels of each of KIM-1, NGAL, and BUN are reduced after administration of the conjugates of the present invention when compared to prior conjugates using currently available peptide carriers.

In some embodiments, the levels of the or each marker/s is significantly reduced when compared to prior conjugates using currently available peptide carriers.

In some embodiments, the levels of the or each marker/s is reduced by up to 5%, 10%, 15%, 20%, 25%, 30%, 35%, 40%, 45%, 50% after administration of the conjugates of the present invention when compared to prior conjugates using currently available peptide carriers.

As provided above, the methods of the invention include administration of 1 mg/kg to 60 mg/kg of a conjugate of an oligonucleotide and a peptide covalently bonded or linked via a linker to the oligonucleotide to the subject (e.g., a subject amenable to exon 51 skipping). In some embodiments, 30 mg/kg to 60 mg/kg (e.g., 40 mg/kg to 60 mg/kg; 30 mg/kg to 50 mg/kg; 30 mg/kg to 40 mg/kg; e.g., 30 mg/kg, 40 mg/kg, 50 mg/kg, or 60 mg/kg) of conjugate is administered. In some embodiments, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, or 45 mg/kg to 60 mg/kg of conjugate is administered. In some embodiments, 1 mg/kg to 30 mg/kg (e.g., 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, or 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of conjugate is administered e.g., 1 mg/kg, 4 mg/kg, 5 mg/kg, 6 mg/kg, 8 mg/kg, 10 mg/kg, 15 mg/kg, 20 mg/kg, 25 mg/kg, or 30 mg/kg) of conjugate is administered. In some embodiments, low doses (e.g., 1 mg/kg to 30 mg/kg) are administered to provide less drug exposure, and thus increase safety, while still maintaining sufficient efficacy.

### Peptide Preparation

Peptides of the invention may be produced by any standard protein synthesis method, for example chemical synthesis, semi-chemical synthesis or through the use of expression systems. Accordingly, the present invention also relates to the nucleotide sequences comprising or consisting of the DNA coding for the peptides, expression systems e.g. vectors comprising said sequences accompanied by the necessary sequences for expression and control of expression, and host cells and host organisms transformed by said expression systems.

Accordingly, a nucleic acid encoding a peptide according to the present invention is also provided.

In some embodiments, the nucleic acids may be provided in isolated or purified form.

An expression vector comprising a nucleic acid encoding a peptide according to the present invention is also provided.

In some embodiments, the vector is a plasmid.

In some embodiments, the vector comprises a regulatory sequence, e.g. promoter, operably linked to a nucleic acid encoding a peptide according to the present invention. In some embodiments, the expression vector is capable of expressing the peptide when transfected into a suitable cell, e.g., mammalian, bacterial, or fungal cell.

A host cell comprising the expression vector of the invention is also provided.

Expression vectors may be selected depending on the host cell into which the nucleic acids of the invention may be inserted. Such transformation of the host cell involves conventional techniques such as those taught in Sambrook et al., Molecular Cloning: A Laboratory Manual, Cold Spring Harbor Laboratory Press, NY, USA, 2001. Selection of suitable vectors is within the skills of the person knowledgeable in the field. Suitable vectors include plasmids, bacteriophages, cosmids, and viruses.

The peptides produced may be isolated and purified from the host cell by any suitable method, e.g., precipitation or chromatographic separation, e.g., affinity chromatography.

Suitable vectors, hosts, and recombinant techniques are well known in the art.

The following examples are meant to illustrate the invention. They are not meant to limit the invention in any way.

### EXAMPLES

### Example 1. Preparation of Conjugates

Conjugates described herein may be prepared using techniques described, e.g., in WO 2020115494 and WO 2020030927.

9-Fluroenylmethoxycarbonyl (Fmoc) protected L-amino acids, benzotriazole-1-yl-oxy-tris-pyrrolidino-phosphonium (PyBOP), 2-(1 H-benzotriazole-1-yl)-1,1,3,3-tetramethyluronium hexafluorophosphate (HBTU), and the Fmoc\hat{ }-Ala-OH preloaded Wang resin (0.19 or 0.46 mmol/g) were obtained from Merck (Hohenbrunn, Germany). HPLC grade acetonitrile, methanol and synthesis grade N-methyl-2-pyrrolidone (NMP) were purchased from Fisher Scientific (Loughborough, UK). Peptide synthesis grade N,N-dimethylformamide (DMF) and diethyl ether were obtained from VWR (Leicestershire, UK). Piperidine and trifluoroacetic acid (TFA) were obtained from Alfa Aesar (Heysham, England). PMO was purchased from Gene Tools Inc. (Philomath, USA). Chicken Embryo Extract and horse serum were obtained from Sera Laboratories International Ltd (West Sussex, UK). Interferon was obtained from Roche Applied Science (Penzberg, Germany). All other reagents were obtained from Sigma-Aldrich (St. Louis, MO, USA) unless otherwise stated. MALDI-TOF mass spectrometry was carried out using a Voyager DE Pro BioSpectrometry workstation. A stock solution of 10 mg/mL of a-cyano-4-hydroxycinnamic acid or sinapinic acid in 50% acetonitrile in water was used as matrix. Error bars are±0.1%.

Peptides were either prepared on a 10 pmol scale using an Intavis Parallel Peptide Synthesizer or on a 100 pmol scale using a CEM Liberty Blue™ Peptide Synthesizer (Buckingham, UK) using Fmoc\hat{ }-Ala-OH preloaded Wang resin (0.19 or 0.46 mmol/g, Merck Millipore) by applying standard Fmoc chemistry and following manufacturer's recommendations. In the case of synthesis using the Intavis

Parallel Peptide Synthesizer, double coupling steps were used with a PyBOP/NMM coupling mixture followed by acetic anhydride capping after each step. For synthesis using the CEM Liberty Blue Peptide Synthesizer, single standard couplings were implemented for all amino acids except arginine, which was performed by double couplings. The coupling was carried out once at 75° C. for 5 min at 60-watt microwave power except for arginine residues, which were coupled twice each. Each deprotection reaction was carried out at 75° C. twice, once for 30 sec and then for 3 min at 35-watt microwave power. Once synthesis was complete, the resin was washed with DMF (3×50 mL) and the N-terminus of the solid phase bound peptide was acetylated with acetic anhydride in the presence of DI PEA. at room temperature. After acetylation of the N-terminus, the peptide resin was washed with DMF (3×20 mL) and DCM (3×20 mL). The peptides were cleaved from the solid support by treatment with a cleavage cocktail consisting of trifluoroacetic acid (TFA): H2O: triisopropylsilane (TIPS) (95%: 2.5%: 2.5%: 3-10 mL) for 3 h at room temperature. After peptide release, excess TFA was removed by sparging with nitrogen. The crude peptide was precipitated by the addition of cold diethyl ether (15-40 mL depending on scale of the synthesis) and centrifuged at 3200 rpm for 5 min. The crude peptide pellet was washed thrice with cold diethyl ether (3×15 mL) and purified by RP-HPLC using a Varian 940-LC HPLC System fitted with a 445-LC Scale-up module and 440-LC fraction collector. Peptides were purified by semi-preparative HPLC on an RP-C18 column (10×250 mm, Phenomenex Jupiter) using a linear gradient of CH₃CN in 0.1% TFA/H2O with a flow rate of 15 mL/min. Detection was performed at 220 nm and 260 nm. The fractions containing the desired peptide were combined and lyophilized to yield the peptide as a white solid.

A PMO antisense sequence for inducing dystrophin exon 51 skipping (5′-CTCCAACATCAAGGAAGATGGCATTTCTAG-3′ (SEQ ID NO: 106)) can be used, e.g., in the preparation of Conjugate 1. A peptide (Ac-RBRRBRFQILYBRBR) may be conjugated to the 3′-end of the PMO through its C-terminal carboxyl group via a glutamic acid linker, where free —COON in the glutamic acid residue was replaced with —CONH₂ to produce the conjugate [peptide]-CO—CH(CONH₂)—CH₂CH₂CO-[3′-oligonucleotide-5′], also identified as the following structure:

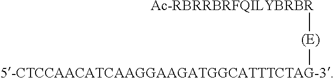

This may be achieved using PyBOP and HOAt in NMP in the presence of DIPEA in DMSO. In some instances, HBTU may be used in place of PyBOP for activation of the C-terminal carboxyl group of the peptide. In general, to a solution of peptide in N-methylpyrrolidone (NMP) may be added PyBOP (in NMP), HOAt in (NMP), diisopropylethyl amine (DIPEA), and PMO (in DMSO). The resulting mixture may be warmed, e.g., to 40° C., and the reaction may be quenched by the addition of TFA in H₂O. This solution may be purified by ion exchange chromatography. For example, the PMO-peptide conjugate may be purified using, e.g., a linear gradient of NaCI (aq.)/CH₃CN/phosphate buffer (pH 7.0). The fractions containing the desired compound may be combined and lyophilized to yield the resulting peptide-PMO. The removal of excess salts from the peptide—PMO conjugate may be afforded through the filtration of the fractions collected after ion exchange using a centrifugal filter device. The conjugate may be lyophilized and analyzed by MALDI-TOF. The conjugate may be dissolved in sterile water and filtered through a cellulose acetate membrane before use. The concentration of peptide-PMO may be determined by the molar absorption of the conjugates at 265 nm in 0.1 N HCl solution.

### Example 2. Non-human Primate Study

Conjugate 1 shown below was reconstituted to 25 mg/mL with 0.9% sterile saline.

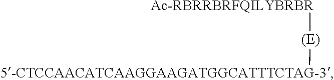

where 5′ group is

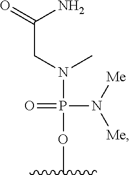 [SMILES: *OP(=O)(N(C)C)N(C)CC(N)=O]

linker (E) is

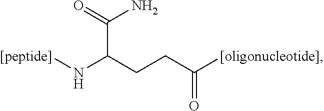

and the oligonucleotide is PMO linked through —P(═O)(NMe₂)—O—.

### NHP Single Infusion Dose Response Study

The efficacy of exon skipping of Conjugate 1 was tested in non-human primates (NHP). Specifically, naïve cynomolgus monkeys aged 2-3 years were administered the conjugate as a single intravenous infusion over 30 minutes at dose levels of 40 mg/kg or 60 mg/kg (n=2 males and n=2 females per group). A control group (n=1 male and n=1 female) received the control item 0.9% sterile saline. Naïve cynomolgus monkeys aged 2-4 years were administered the conjugate by a single intravenous slow bolus injection (1-2 minutes) at 20 mg/kg, 40 mg/kg, or 60 mg/kg (n=1 male and n=1 female per group).

Animals were sacrificed one-week post administration. At scheduled necropsy section of tissue (biceps, gastrocnemius, quadriceps, soleus, tibialis anterior, diaphragm, lung, heart, cerebral cortex cerebellum, colon, duodenum, esophagus, epidermal skin, adrenal gland, thyroid gland, kidney, liver, spleen, pancreas, thymus, testes, and ovaries) were collected for exon skipping analysis and tissue bioanalysis. Terminal liver and kidney samples were subject to gross necropsy. During the in-life blood samples were analyzed for clinical chemistry, hematology, complement, and coagulation on days 1, 4, and 7 post-dose and compared to pre-dose levels. Urine was additionally collected on days 1, 4, and 7 post-dose and analyzed for KIM-1 and compared to pre-dose levels. Body and organ weight profiles were also assessed.

TABLE 1
Study design.

| | | Dose | Number | |
|---|---|---|---|---|
| Group | Test | level | per group | Administration |
| no. | material | (mg/kg) | (M/F) | style |
| 1 | 0.9% saline | 0 | 1/1 | 30-minute IV infusion |
| 2 | Conjugate 1 | 40 | 2/2 | 30-minute IV infusion |
| 3 | Conjugate 1 | 60 | 2/2 | 30-minute IV infusion |
| 4 | Conjugate 1 | 20 | 1/1 | 1-2 minute IV bolus |
| 5 | Conjugate 1 | 40 | 1/1 | 1-2 minute IV bolus |
| 6 | Conjugate 1 | 60 | 1/1 | 1-2 minute IV bolus |

### Safety Assessment

Clinical chemistry, hematology, complement, and coagulation were assessed on days 1, 4, and 7 post-dose and compared to pre-dose levels. Body and organ weight profiles were also assessed.

Clinical chemistry markers used in this study are listed in Table 2.

TABLE 2
Clinical Chemistry Parameters

| | |
|---|---|
| Alanine aminotransferase | Albumin |
| Aspartate aminotransferase | Globulin (calculated) |
| Alkaline phosphatase | Albumin/globulin ratio |
| Gamma-glutamyltransferase | Glucose |
| Total bilirubin | Cholesterol |
| Urea nitrogen | Triglycerides |
| Creatinine | Sodium |
| Calcium | Potassium |
| Phosphorus | Chloride |
| Total protein | Creatine kinase |

Hematology markers used in this study are summarized in Table 3.

TABLE 3
Hematology Parameters

| | |
|---|---|
| Red blood cell count | Platelet count |
| Hemoglobin concentration | White blood cell count |
| Hematocrit | Neutrophil count |
| Mean corpuscular volume | Lymphocyte count |
| Red blood cell distribution width | Monocyte count |
| Mean corpuscular hemoglobin concentration | Eosinophil count |
| Mean corpuscular hemoglobin | Basophil count |
| Reticulocyte count | Large unstained cells count |

Complement markers used in this study are summarized in Table 4.

TABLE 4

| | |
|---|---|
| C3a | SC5b-9 |
| Bb | C5a |
| CH50 | |

Coagulation markers used in this study are summarized in Table 5.

TABLE 5
Coagulation Parameters

| | |
|---|---|
| Activated partial thromboplastin time | Prothrombin time |
| Fibrinogen | Sample quality |

Additionally, KIM-1 and creatinine urinalysis was performed on samples collected pre-dose and post-dose on days 1, 4, and 7.

### RT—PCR Analysis

The level of exon 51 skipping was determined by RT—PCR. Skeletal, cardiac, and smooth muscle tissue was homogenized using a bead-based homogenization method. RNA was extracted using a Maxwell RSC 48 instrument (Promega) and a simplyRNA Tissue Kit (Promega) according to the manufacturer's recommendations. Concentration and purity of the RNA was determined using a

ClarioStar (BMG LabTech). Quantified RNA was reverse transcribed using a High-Capacity cDNA Reverse Transcription Kit (ThermoFisher Scientific, 4368813), under the thermal cycling conditions described in Table 6.

TABLE 6
Reverse transcription thermal cycling conditions.
Thermal Cycling Conditions

| | Temperature | Time |
|---|---|---|
| Step | (° C.) | (min) |
| 1 | 25 | 10 |
| 2 | 37 | 120 |
| 3 | 85 | 5 |
| 4 | 4 | Hold |

A nested—PCR was performed as 2 consecutive PCR reactions. The first PCR was performed using the reverse transcribed cDNA template. The second PCR was performed using product from the first PCR. All primers used in for PCR reactions are identified in Table 7, and thermal cycling conditions are outlined in Table 8. Final PCR products were analyzed by agarose (2%) gel electrophoresis. Gels were prepared using Midori Green Advance Stain (Nippon Genetics). HyperLadder 50 bp (Bioline, BIO-33039) and PCR product were loaded on the agarose gel and run until an appropriate degree of band separation was achieved. Subsequently gel image acquisition was performed on resolved gels using a G:BOX (Syngene) gel imaging system. Unskipped/native and skipped/Aex51 bands from nested—PCR gels were subjected to densitometry analysis using ImageJ software (Fiji). Densitometry values from band quantification were used to determine nhpDMD exon 51 skipping, using the below formula: nhpDMD exon 51 skipping formula: ([peak area of skipped fragment]/[peak area of skipped fragment+peak area of unskipped fragment])×100.

TABLE 7
Primers and primer sequences.

| | PCR | | |
|---|---|---|---|
| | Primers | Exon | Sequence |
| | Name | target | (5′-3′) |
| PCR-1 | nhpDMDex48 | Ex48 | TGCTCCTGTGGCT |
| | (F) | | GTCTCCT |
| | nhpDMDex53 | Ex53 | AGCTTGGCTCTGG |
| | (R) | | CCTGTCCT |
| PCR-2 | nhpDMDex49 | Ex49 | ACCAGCCACTCAG |
| | (F) | | CCAGTGA |
| | nhpDMDex52 | Ex52 | GATTGTTCTAGCC |
| | (R) | | TCTTGATTGC |

TABLE 8
Thermal cycling conditions.

| Step | Temperature (° C.) | Time | | Cycles |
|---|---|---|---|---|
| Initial | 95 | 5 | min | 1 |
| denaturing | | | | |
| Denaturing | 95 | 25 | s | 20 |
| Annealing | 62 | 35 | s | |
| Extension | 72 | 65 | s | |
| Final | 72 | 5 | min | 1 |
| extension | | | | |
| Hold | 4 | N/A | | N/A |
| Initial | 95 | 5 | min | 1 |
| denaturing | | | | |
| Denaturing | 95 | 25 | s | 32 |
| Annealing | 58 | 35 | s | |
| Extension | 72 | 65 | s | |
| Final | 72 | 5 | min | 1 |
| extension | | | | |

| | | | |
|---|---|---|---|
| Hold | 4 | N/A | N/A |

Exon 51 skipping efficiency in the non-human primates receiving a bolus or an infusion of Conjugate 1 is summarized in Table 9.

TABLE 9
Exon 51 skipping efficiency of Conjugate 1

| | |
|---|---|
| 40 mg/kg | 60 mg/kg |

| | | | | | |
|---|---|---|---|---|---|
| Bolus | Infusion | Difference | Bolus | Infusion | Difference |
| (%) | (%) | (%) | (%) | (%) | (%) |

Tibialis Anterior

| | | | | | |
|---|---|---|---|---|---|
| 61.8 | 73.5 | 19 | 75.7 | 87.5 | 16 |

Diaphragm

| | | | | | |
|---|---|---|---|---|---|
| 88.1 | 88.3 | 0 | 91.8 | 95.8 | 4 |

Heart

| | | | | | |
|---|---|---|---|---|---|
| 45.8 | 33.0 | −28 | 86.7 | 74.2 | −14 |

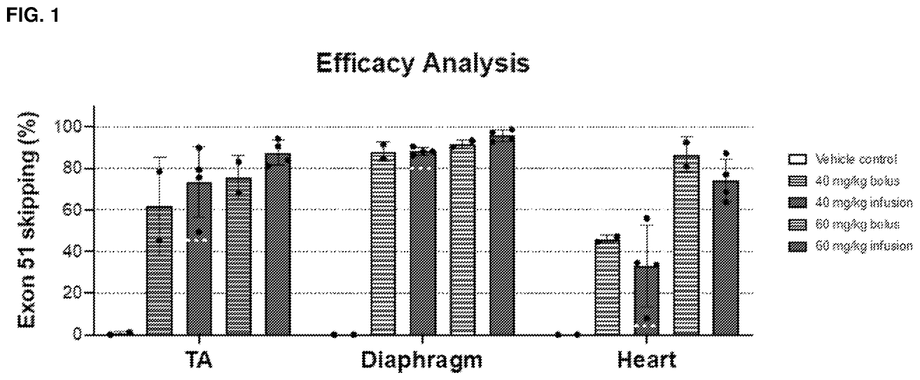

The results are illustrated in FIGS. 1-13E.

FIG. 1 demonstrates the dose response in key muscle groups in animals (n=2-4).

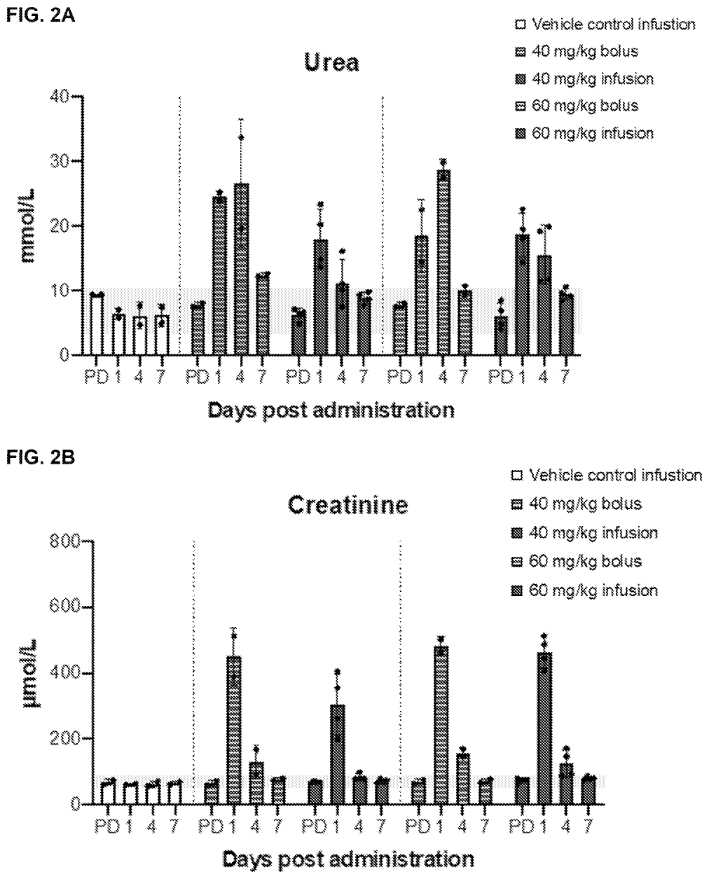

FIGS. 2A and 2B demonstrate that post-dosing elevations of urea and creatinine (kidney function markers) are transient. Elevations seen with infusion administration are lower than the levels seen previously by bolus administration. All elevations return to pre-dose and/or control levels by day 7 post-administration.

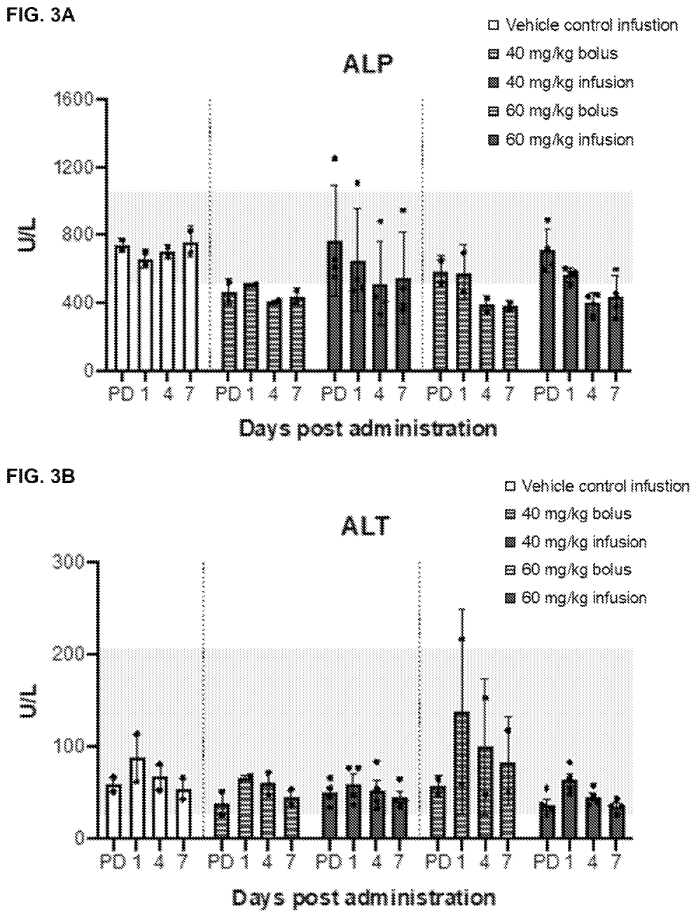

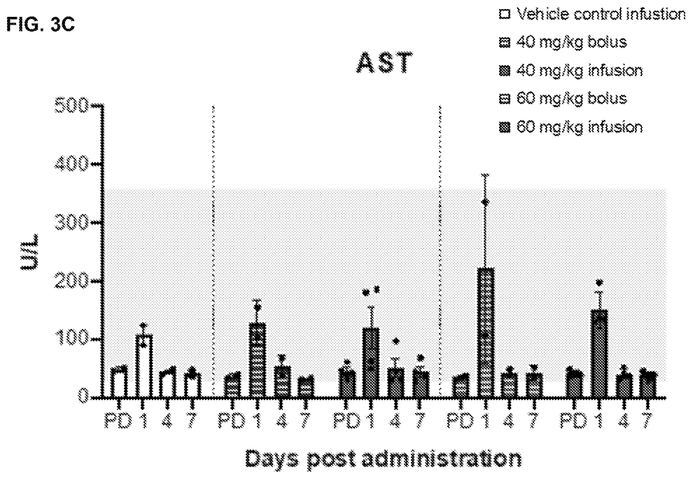

FIGS. 3A, 3B, and 3C demonstrate that ALP and ALT profiles are benign with infusion administration. AST elevations seen with infusion administration may not be test item related and are within historic control min/max ranges. Lower levels are observed than what was seen previously by bolus and return to pre-dose levels by day 4 post-admin.

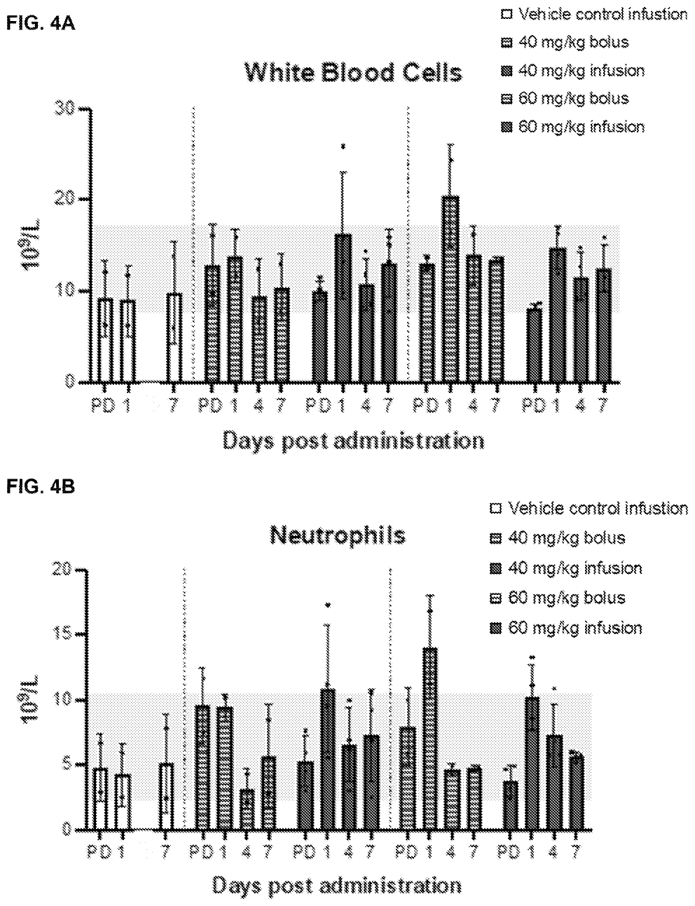

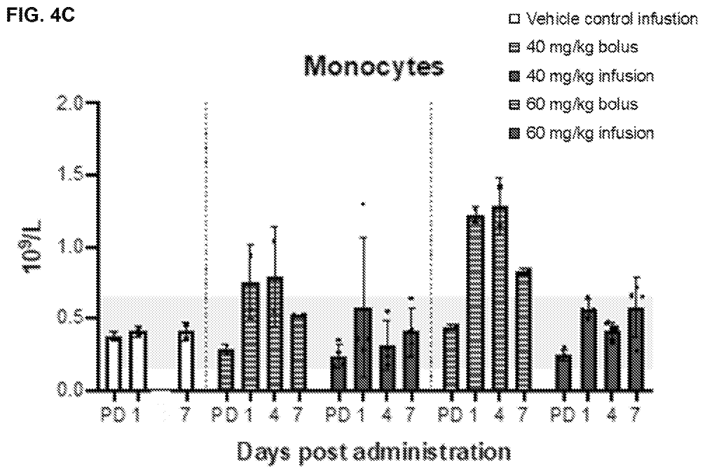

FIGS. 4A, 4B, and 4C demonstrate that minor fluctuations present in primary hematology parameters post-dosing of Conjugate 1 do not reach biologically significant levels. Primary hematology parameters for infusion administration have the same or lower profiles than what was seen with bolus administration.

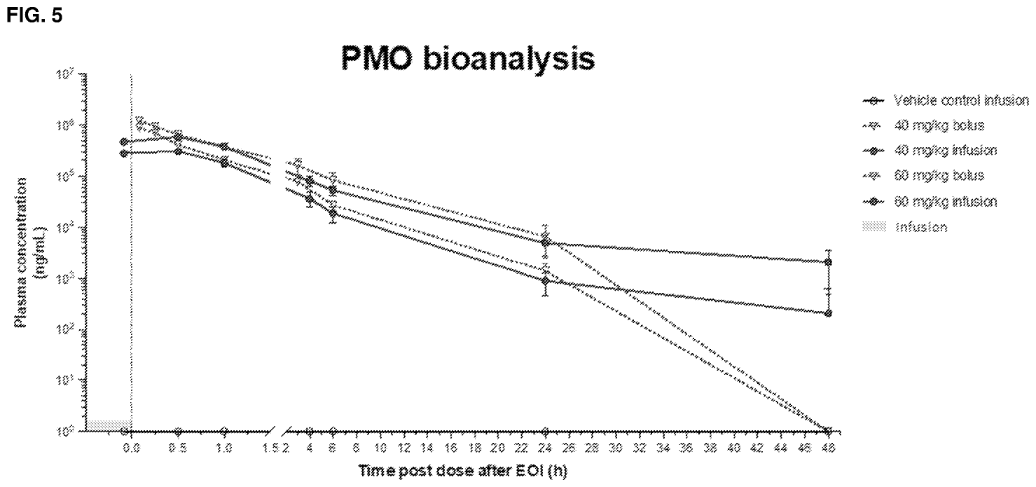

FIG. 5 demonstrates that infusion administration resulted in slightly lower plasma concentrations than bolus administration but a longer presence of the conjugate in plasma (48 h post-dosing). Duration of infusion was 30 minutes.

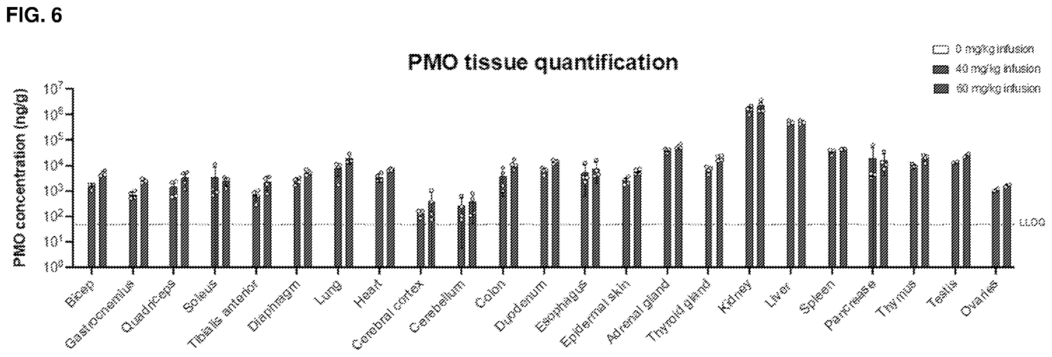

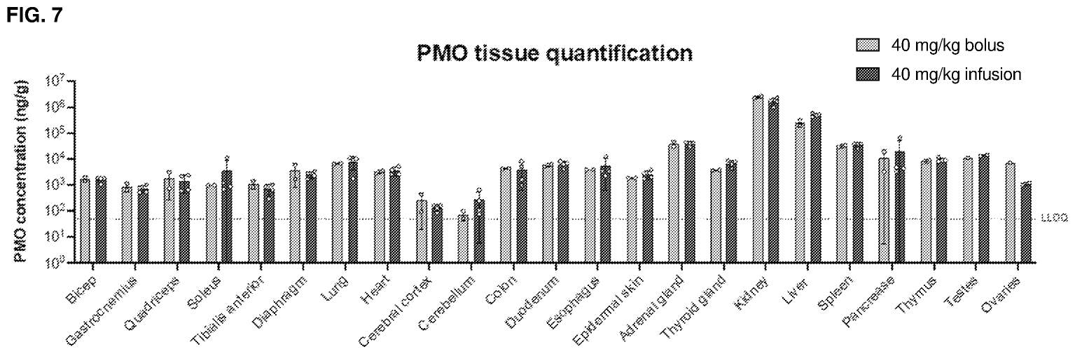

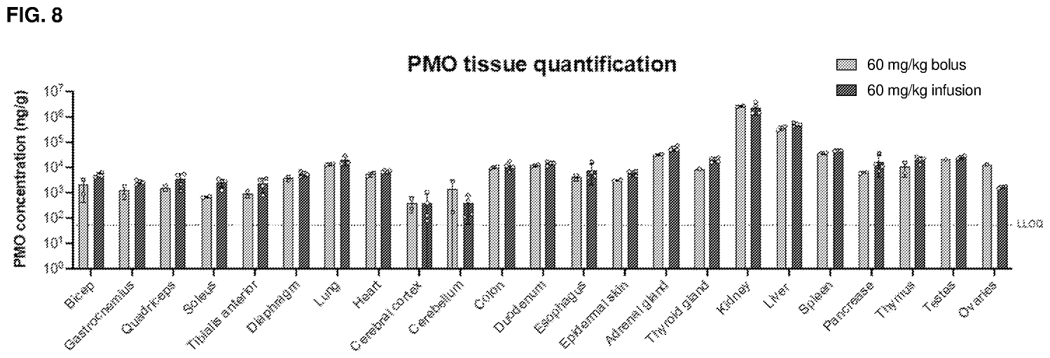

FIG. 6 demonstrates dose response with global delivery of Conjugate 1 detected in traditionally hard to reach tissues, e.g., heart and CNS. PMO was detected 7 days post-dosing. FIGS. 7 and 8 demonstrate that infusion administration results in similar or better levels of PMO concentration and presence in tissue over bolus administration.

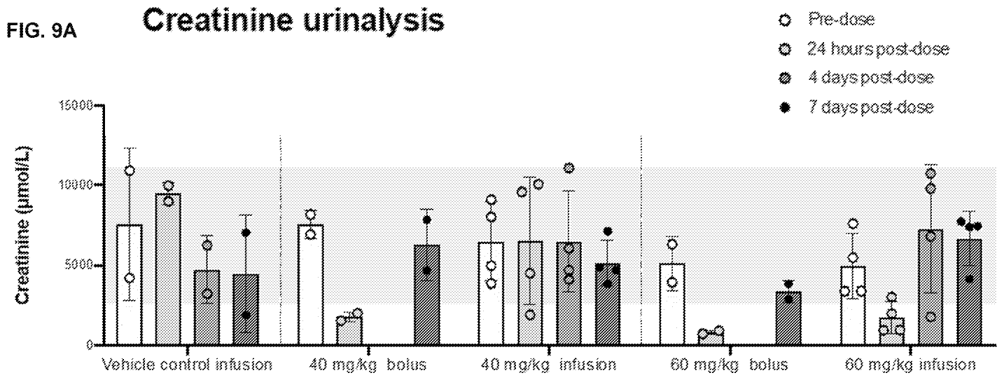

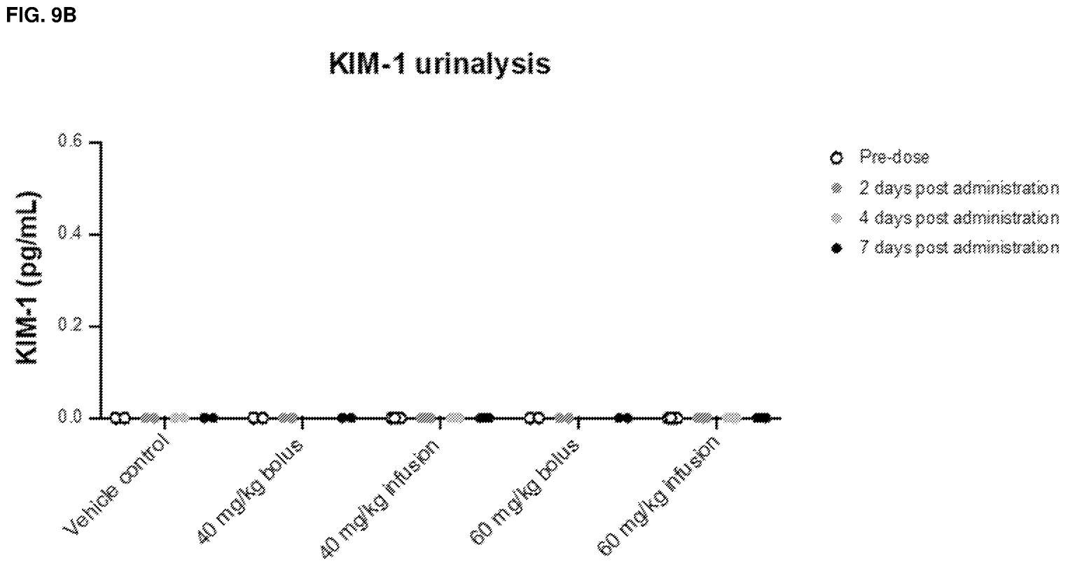

While variability between animals renders the data interpretation more complicated for creatinine urinalysis, it can be seen in FIG. 9A that transient modulation present with 60 mg/kg infusion returns to vehicle control levels by day 4 post-dose. Additionally, KIM-1 (urinary biomarker for kidney damage) urinalysis revealed no detectable levels of KIM-1 at any time point for any animal during the study (urine was collected pre-dose, 24 hours post-dose and 4 and 7 days post-dose and analyzed for KIM-1 levels; FIG. 9B).

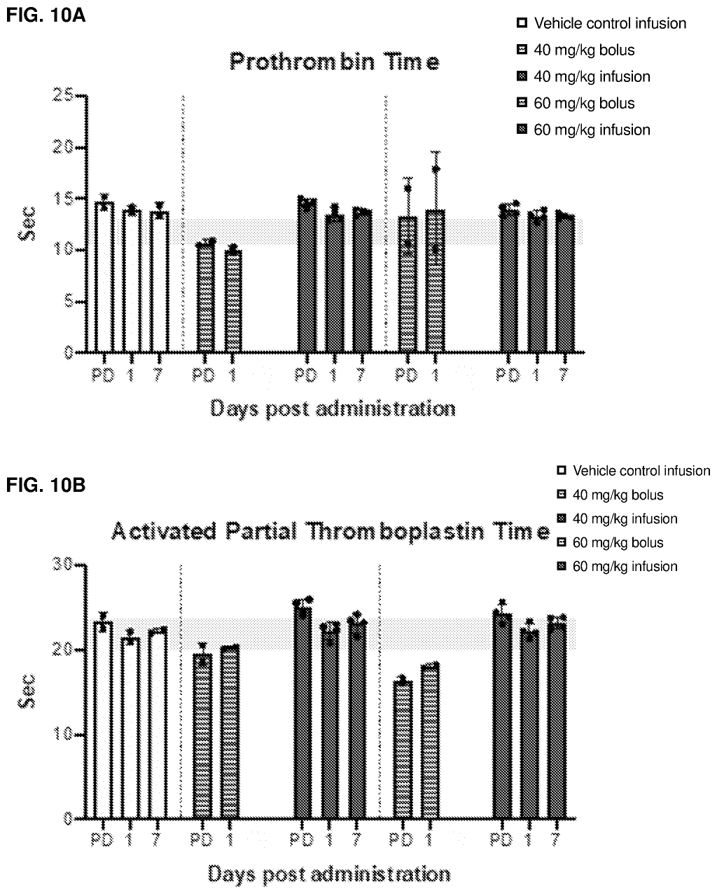

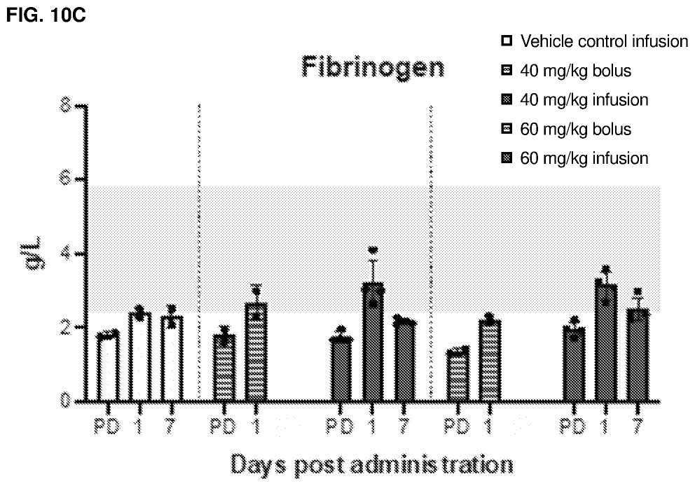

FIGS. 10A, 10B, and 10C demonstrate that coagulation markers remain unaffected with bolus or infusion administration.

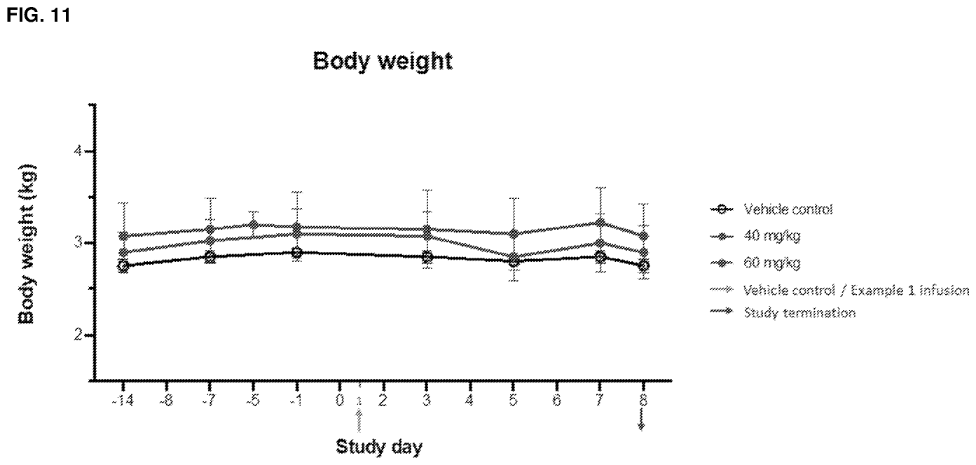

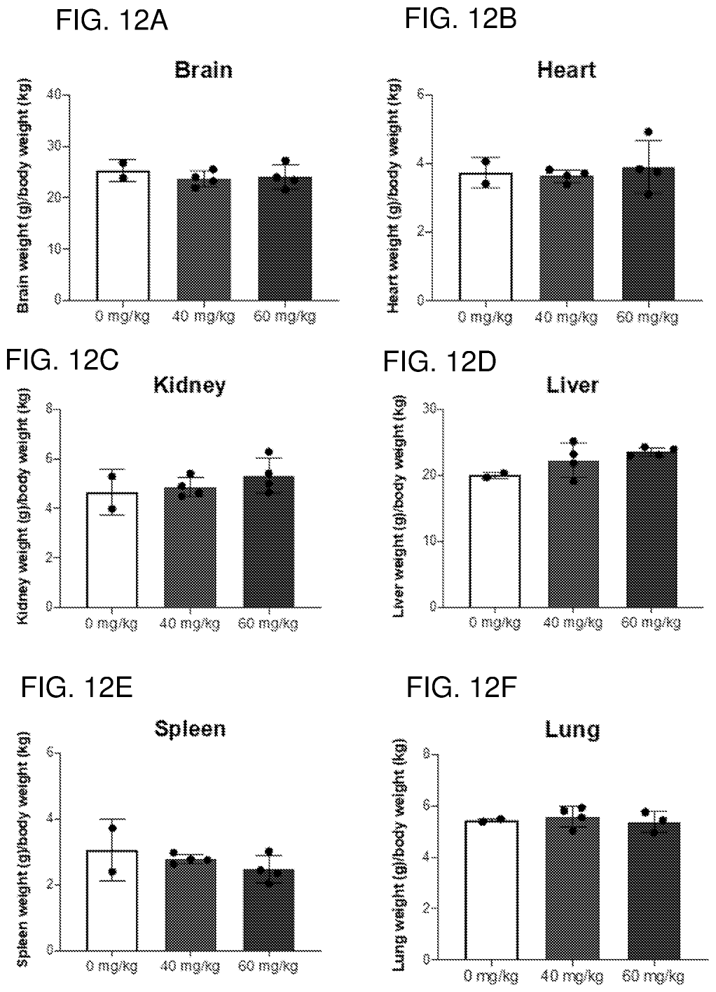

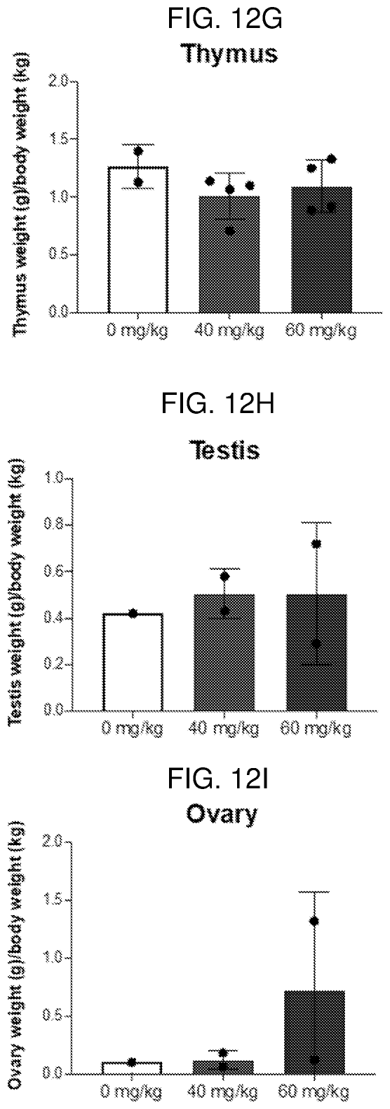

FIG. 11 demonstrates that infusion of Conjugate 1 had no impact on body weight profiles. No treatment-related organ weight changes were observed in the animals receiving Conjugate 1 (FIGS. 12A-12I). Occasional organ weight differences observed were considered incidental and/or related to difference of sexual maturity and unrelated to administration.

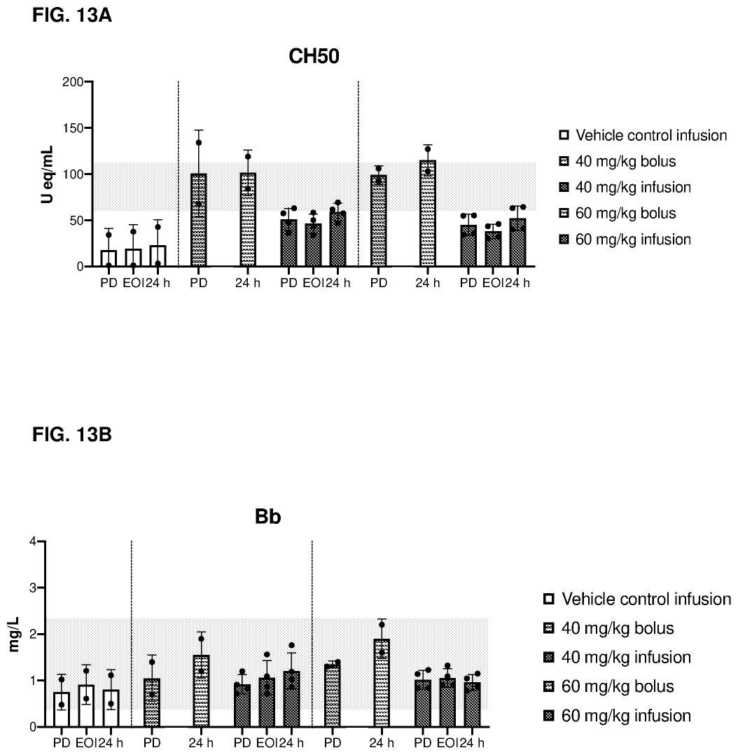

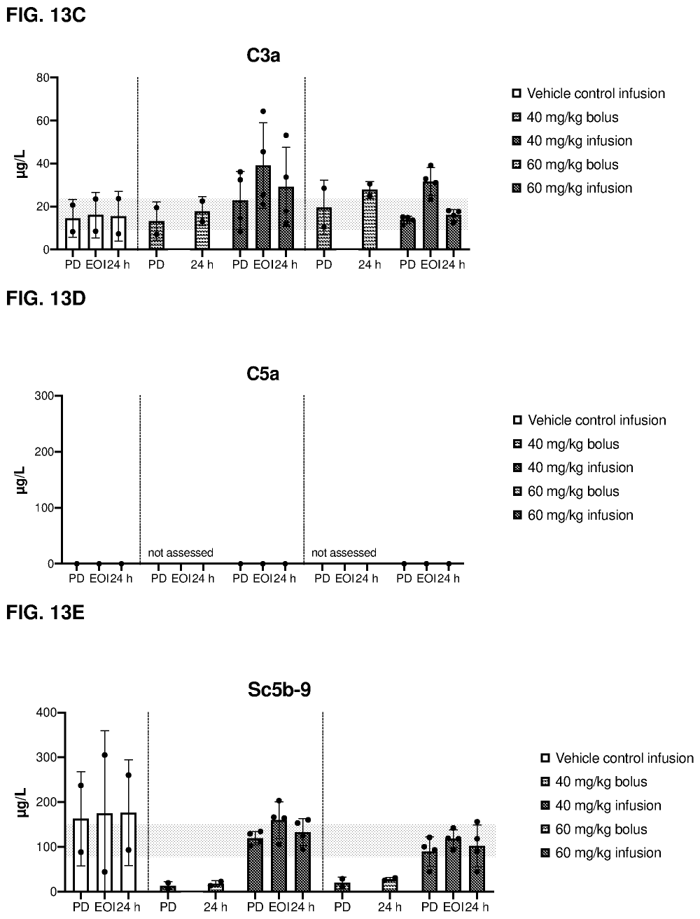

FIGS. 13A-13E demonstrate that serum complement activation in CH₅₀, Bb, C3a or SC5b-9. C5a was not detected during the study.

A summary of the conclusions is as follows.

No mortalities, no SAEs and no AEs observed with infusion of Conjugate 1 at 40 or 60 mg/kg.

No macroscopic tissue pathology observed at either dose with infusion of Conjugate 1.

Majority of microscopic findings are considered not adverse (benign), not degenerative and known class effects of oligos. Main microscopic findings noted in the kidneys at 40 mg/kg or 60 mg/kg included: minimal to moderate tubular basophilia, minimal to mild tubular basophilic granules, minimal to mild tubular dilatation at 60 mg/kg and minimal multifocal hyaline casts.

All doses observed transient, recoverable, mild elevation of some haematology markers that can be considered benign.

All doses permit delivery to tissues integral for addressing the DMD pathology.

All doses observed transient, recoverable, mild elevation of some clinical chemistry markers.

No impact from Conjugate 1 on organ or body weight.

### Example 3. Additional NHP Studies

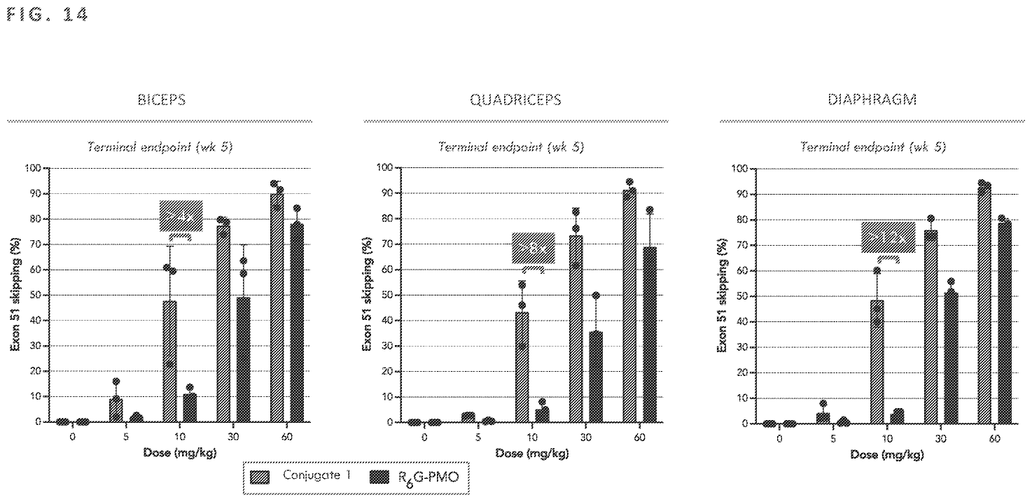

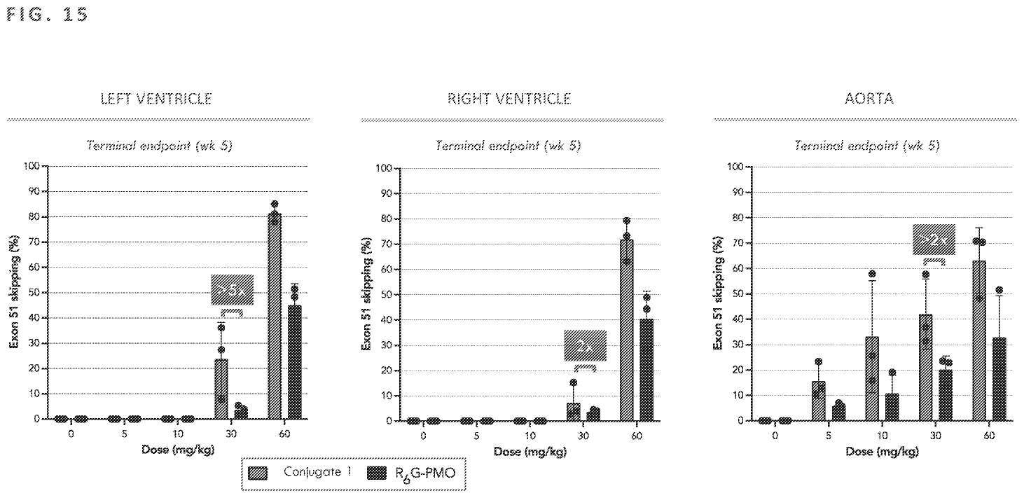

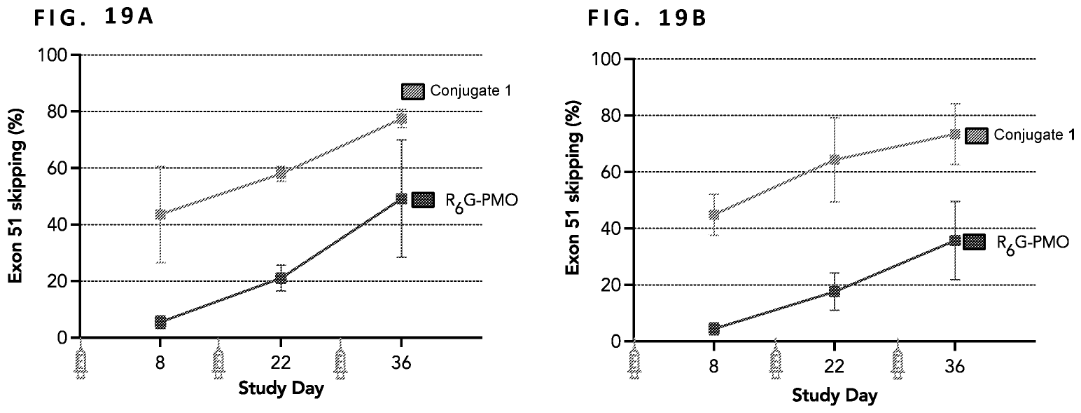

In additional studies, naïve cynomolgus monkeys were administered the conjugate described above (Conjugate 1) or a different conjugate, including the same oligonucleotide sequence as Conjugate 1 but linked to a peptide consisting of the sequence (Arg)₆Gly. The conjugates were administered by IV infusion over 30 minutes at 5, 10, 30, or 60 mg/kg (n=3). Q2W, three doses were administered with saline being used as a control. Biopsies were taken 7 days after each administration, and issues were harvested 7 days after the final administration. The levels of exon 51 skipping (%) in biceps, quadriceps, and diaphragm 7 days after final administration are shown in FIG. 14, while the levels of exon 51 skipping (%) in heart tissue (left ventricle, right ventricle, and aorta) 7 days after final administration are shown in FIG. 15. As is shown in these figures, Conjugate 1 showed dramatically improved efficacy in exon 51 skipping over the (Arg)6Gly conjugate in the tissues shown in the figures. In addition, it is of note that Conjugate 1 showed substantial efficacy at a dose as low as, e.g., 10 mg/kg in certain tissues. Analysis of the biopsy samples taken 7 days after the first and second administrations, and the terminal samples taken 7 days after the final administration showed that exon skipping accumulated with repeat dose administration of Conjugate 1. Results are shown in FIGS. 19A and 19B.

### Methods

RNA was extracted, quantified, and then normalized to 50 ng/μL with nuclease-free water. Subsequently, 500 ng of normalized RNA was reverse transcribed. Primers were prepared as 10 uM aliquots from 100 μM stocks. PCR master mix, comprising 2× DreamTaq Master Mix, primers, and nuclease-free water, was vortexed and briefly centrifuged after thawing.

TABLE 10
PCR Primers

| Exon target | Sequence (5′-3′) |
|---|---|
| Ex48 | CCAGCCACTCAGCCAGTGAAG |
| Ex53 | CGATCCGTAATGATTGTTCTAGCC |

TABLE 11
PCR Reaction (per sample)

| Reagent | Volume (μL) | Final Concentration |
|---|---|---|
| 2X DreamTaq Master Mix | 12.5 | 1X |
| Forward primer, 10 μM | 1.25 | 0.5 μM |
| Reverse primer, 10 μM | 1.25 | 0.5 μM |
| Nuclease free water | 7.0 | — |
| Template cDNA | 3 | — |
| Total volume | 25 | — |

22 μl of reaction master mix was added to each thin-walled PCR reaction tube. Then 3 μl of template cDNA for each sample was added to each tube and thoroughly mixed. Samples were gently vortexed and PCR tubes were briefly spun down, with care taken to eliminate any bubbles prior to proceeding. Tubes were then placed in Proflex and PCR was run with the following thermal cycling conditions:

TABLE 12
PCR thermal cycling conditions

| Step | Temperature (° C.) | Time | | Cycles |
|---|---|---|---|---|
| Initial denaturing | 94 | 2 | minutes | 1 |
| Denaturing | 94 | 30 | seconds | 34 |
| Annealing | 55 | 30 | seconds | |
| Extension | 68 | 1 | minute | |
| Final extension | 68 | 5 | minutes | 1 |

| | | | |
|---|---|---|---|
| Hold | 4 | ∞ | N/A |

After PCR, samples were either put on ice or frozen at −20° C. When ready for processing samples were fractionated on a 2% agarose gel, which was then imaged. Bands were analyzed as follows:

TABLE 13

| Sample | Expected band size (bp) |
|---|---|
| Control (C) RNA | 478 |
| No template control (NTC) | No band(s) |
| Unskipped (native ex51) | 478 |
| Skipped (Δex51) | 246 |

Bands were quantified by densitometry using ImageJ software. The ‘plot lanes’ command was used, and the line tool was used to mark the base of the curve. The wand tool was used to select the area under the curve to obtain the area. Percentage of nhpDMDex51 skipping was determined by:

$$\frac{Unskipped ( native e \times 51 )}{Unskipped ( native e \times 51 ) + Skipped ( Δ e \times 51 )} \times 100$$

### Example 4. A single dose study in mdx mice

A single dose of conjugate 2 was administered intravenously to mdx mice; serum creatine kinase levels were then measured 7 days after the injection. Vehicle (0.9% saline) was used as a control agent in this study. Wild-type (WT) mice were used as control animals in this study. R6G—PMO was used as a comparator conjugate. R6G—PMO consists of the same oligonucleotide sequence as Conjugate 2 but linked to a peptide consisting of the sequence (Arg)6Gly. The results are shown in FIGS. 16, 17A, 17B, 18A, and 18B.

Conjugate 2 is shown below:

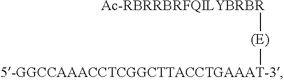

where 5′ group is

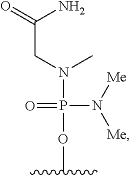 [SMILES: *OP(=O)(N(C)C)N(C)CC(N)=O]

linker (E) is

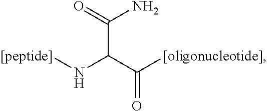

and the oligonucleotide is PMO linked through —P(═O)(NMe₂)—O—.

The results in FIG. 16 suggest that Conjugate 2 is preventing further muscle damage following the administration of a tolerable, single dose of Conjugate 2, as shown by determination of normalized creatine kinase levels.

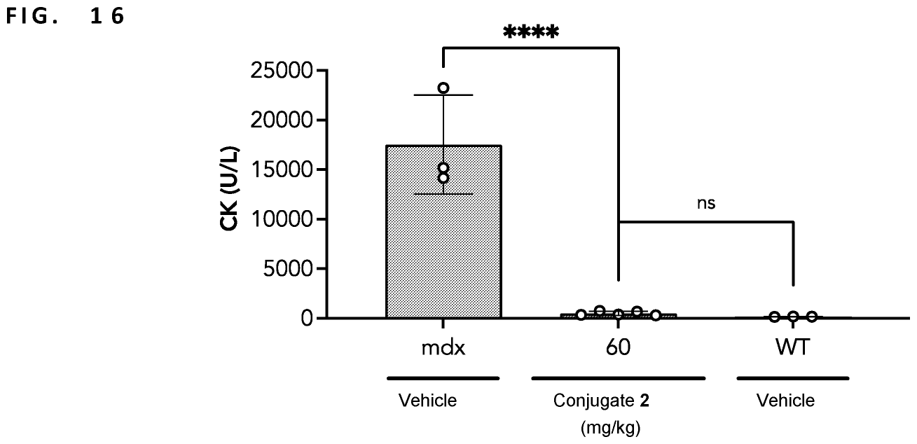

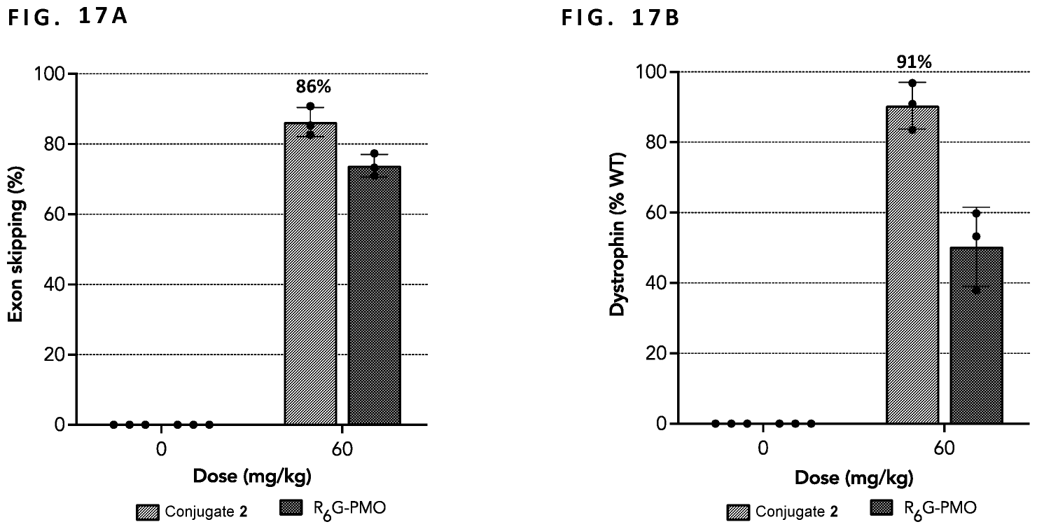

The results in FIGS. 17A and 17B demonstrate that 91% dystrophin restoration is achieved in the quadriceps 7 days after a single dose of Conjugate 2.

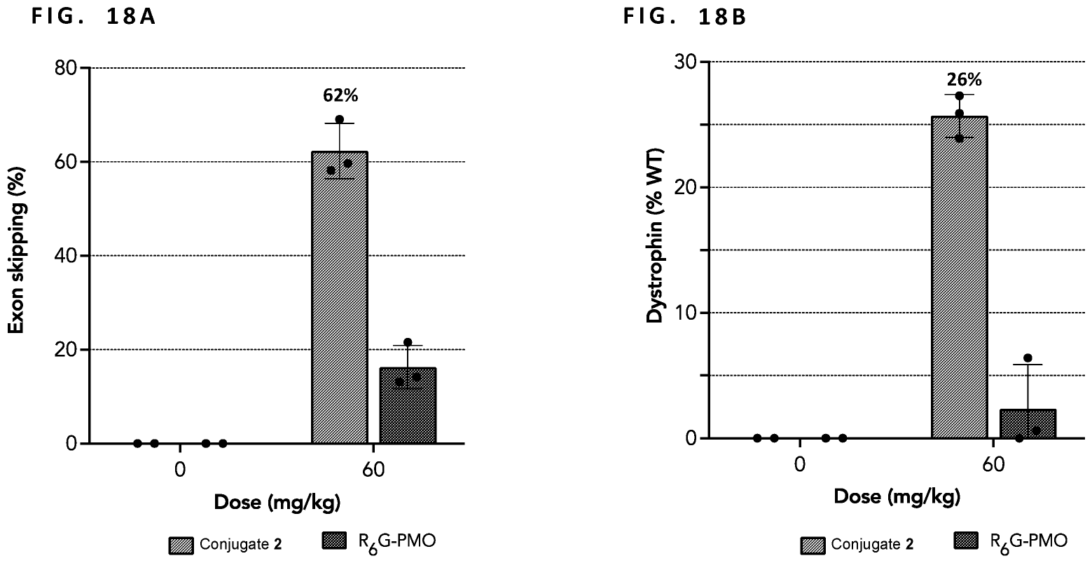

The results in FIGS. 18A and 18B demonstrate that 26% dystrophin restoration is achieved in the heart 7 days after a single dose of Conjugate 2.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Some embodiments are within the scope of the following numbered paragraphs.

1. A method of treating a subject having Duchenne muscular dystrophy, the method comprising administering 1 mg/kg to 60 mg/kg of a conjugate of an oligonucleotide and a peptide covalently bonded or linked via a linker to the oligonucleotide, the peptide comprising at least one cationic domain comprising at least 4 amino acid residues and at least one hydrophobic domain comprising at least 3 amino acid residues, provided that the peptide comprises a total of 7 to 40 amino acid residues, and provided that the at least one cationic domain comprises a beta-alanine residue in combination with arginine and/or histidine residues; and

the oligonucleotide comprising a total of 12 to 40 contiguous nucleobases, wherein at least 12 contiguous nucleobases are complementary to a target sequence in a human dystrophin gene.

2. The method of paragraph 1, wherein the oligonucleotide comprises a sequence selected from the group consisting of:

(SEQ ID NO: 106)
5′-CTCCAACATCAAGGAAGATGGCATTTCTAG-3′;
or
(SEQ ID NO: 107)
5′-ACCAGAGUAACAGUCUGAGUAGGAGC-3′;
(SEQ ID NO: 108;
5′-CUCAUACCUUCUGCUUGAUGAUC-3′;
(SEQ ID NO: 109)
5′-UUCUGUCCAAGCCCGGUUGAAAUC-3′;
(SEQ ID NO: 110)
5′-ACAUCAAGGAAGAUGGCAUUUCUAGUUUGG-3′;
(SEQ ID NO: 111)
5′-ACAUCAAGGAAGAUGGCAUUUCUAG-3′;
(SEQ ID NO: 112)
5′-CUCCAACAUCAAGGAAGAUGGCAUUUCUAG-3′;
(SEQ ID NO: 113)
5′-AUCAUUUUUUCUCAUACCUUCUGCUAG-3′;
(SEQ ID NO: 114)
5′-AUCAUUUUUUCUCAUACCUUCUGCUAGGAGCUAAAAAG-3′;
(SEQ ID NO: 115)
5′-CACCCACCAUCACCCUCUGUG-3′;
(SEQ ID NO: 116)
5′-AUCAUCUCGUUGAUAUCCUCAA-3′;

and their thymine-substitution analogues

3. The method of paragraph 1 or 2, wherein each cationic domain has length of between 4 and 12 amino acid residues.

4. The method of paragraph 3, wherein each cationic domain has length of between 4 and 7 amino acid residues.

5. The method of any one of paragraphs 1 to 4, wherein each cationic domain comprises at least 55%, at least 60%, at least 65% at least 70%, at least 75%, at least 80%, at least 85%, at least 90%, or at least 95% cationic amino acids.

6. The method of any one of paragraphs 1 to 4, wherein each cationic domain comprises at least 40%, at least 45%, at least 50%, at least 55%, at least 60%, at least 60%, at least 65%, at least 70% arginine and/or histidine residues.

7. The method of any one of paragraphs 1 to 4, wherein each cationic domain comprises one of the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19) or any combination thereof.

8. The method of any one of paragraphs 1 to 4, wherein each cationic domain comprises or consists of one the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19) or any combination thereof.

9. The method of any preceding paragraph, wherein the peptide comprises two cationic domains.

10. The method of any preceding paragraph, wherein each hydrophobic domain has a length of 3 to 6 amino acids, preferably each hydrophobic domain has a length of 5 amino acids.

11. The method of any preceding paragraph wherein each hydrophobic domain comprises at least 70%, at least 75%, at least 80%, at least 85%, at least 90%, at least 95%, or 100% hydrophobic amino acids.

12. The method of any preceding paragraph, wherein each hydrophobic domain comprises phenylalanine, leucine, Isoleucine, tyrosine, tryptophan, proline, and/or glutamine residues; preferably wherein each hydrophobic domain comprises or consists of phenylalanine, leucine, isoleucine, tyrosine, tryptophan, proline, and/or glutamine residues.

13. The method of any preceding paragraph, wherein the peptide comprises one hydrophobic domain.

14. The method of any preceding paragraph, wherein the or each hydrophobic domain comprises one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26) or any combination thereof.

15. The method of any preceding paragraph, wherein the or each hydrophobic domain comprises or consists of one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26) or any combination thereof.

16. The method of any preceding paragraph, wherein the peptide comprises or consists of two cationic domains and one hydrophobic domain.

17. The method of any preceding paragraph, wherein the peptide comprises or consists of one hydrophobic core domain flanked by two cationic arm domains.

18. The method of any one of paragraphs 1 to 4, wherein the peptide comprises or consists of one hydrophobic core domain comprising a sequence selected from: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26), flanked by two cationic arm domains each comprising a sequence selected from: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19).

19. The method of any one of paragraphs 1 to 4, wherein the peptide comprises or consists of one of the following sequences: RBRRBRRFQILYRBRBR (SEQ ID NO: 27), RBRRBRRYQFLIRBRBR (SEQ ID NO: 31), RBRRBRRILFQYRBRBR (SEQ ID NO: 32), RBRRBRFQILYBRBR (SEQ ID NO: 35), RBRRBRRFQILYRBHBH (SEQ ID NO: 37), RBRRBRRFQILYHBHBR (SEQ ID NO: 38), and RBRRBRFQILYRBHBH (SEQ ID NO: 44).

20. The method of any one of paragraphs 1 to 4, wherein the peptide has the following amino acid sequence RBRRBRFQILYBRBR (SEQ ID NO: 35).

21. The method of any one of paragraphs 1 to 4, wherein the peptide has the following amino acid sequence RBRRBRRFQILYRBHBH (SEQ ID NO: 37).

22. The method of any one of paragraphs 1 to 4, wherein the peptide has the following amino acid sequence RBRRBRFQILYRBHBH (SEQ ID NO: 44).

23. The method of any one of paragraphs 1 to 22, wherein the peptide is bonded to the rest of the conjugate through its N-terminus.

24. The method of paragraph 23, wherein the C-terminus of the peptide is —NH₂.

25. The method of any one of paragraphs 1 to 22, wherein the peptide is bonded to the rest of the conjugate through its C-terminus.

26. The method of paragraph 25, wherein the peptide is acylated at its N-terminus.

27. The method of any preceding paragraph, wherein the conjugate comprises or is of the following structure:

[peptide]-[linker]-[oligonucleotide]

28. The method of any one of paragraphs 1 to 26, wherein the conjugate comprises or is of the following structure:

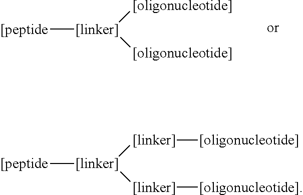

29. The method of any one of paragraphs 1 to 26, wherein the conjugate comprises or is of the following structure:

[peptide]—[linker]—[peptide]—[linker]—[oligonucleotide].

30. The method of any preceding paragraph, wherein each linker is independently of formula (I):

T₁—(CR¹R²)ₙ—T₂. (I)

wherein

T₁ is a divalent group for attachment to the peptide and is selected from the group consisting of —NH— and carbonyl;

T₂ is a divalent group for attachment to an oligonucleotide and is selected from the group consisting of —NH— and carbonyl;

n is 1, 2 or 3;

each R¹ is independently —Y¹ —X¹ —Z¹,

wherein
Y¹ is absent or —(CR^{A1}R^{A2})ₘ—, wherein m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C (O)O—, —C(O)—N (R^{A3})—, —N (R^{A3})C (O)N (R^{A3})—, —N(R^{A3})C(N R^{A3})N (R^{A3})—, —SO—, —S—, —SO₂—, —S(O)₂N(R^{A3})—, or —N(R^{A3})—SO₂—, wherein each R^{A3} is independently selected from hydrogen and methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl,

wherein each (1-6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4} R^{A5}, and (1-4C)alkoxy, wherein R^{A4} and R^{A5} are each independently selected from the group consisting of hydrogen and (1-4C)alkyl; and

each R² is independently —Y²—X²—Z², wherein
Y² is absent or a group of the formula —[CR^{B1} R^{B2}]ₙ— in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})—, or —N(R^{B3})—SO₂—, wherein each R^{B3} is independently selected from hydrogen or methyl; and
Z² is selected from hydrogen, (1-6C)alkyl, (2-6C)alkenyl, (2-6C)alkynyl, aryl, (3-6C)cycloalkyl, (3-6C)cycloalkenyl or heteroaryl, wherein each (1-6C)alkyl, (2- 6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4} R^{B5}, and (1-4C)alkoxy, wherein R^{B4} and R^{B5} are each independently hydrogen or (1-2C)alkyl; with the proviso that; when n=1 and T₁ and T₂ are different to one another, then R¹ and R² are not both H; when n=1, T₁ and T₂ are different to one another and one of R¹ and R² is H then the other of R¹ and R² is not methyl; or when n=2 and each occurrence of R¹ and R² is H, then T₁ and T₂ are both —C(O)- or are both —NH—.

31. The method of paragraph 30, wherein T₂ is —C(O)—.

32. The method of paragraph 30 or 31, wherein each R¹ is independently —Y¹ —X¹ —Z¹, wherein:

Y¹ is absent or —(CR^{A1}R^{A2})ₘ—, wherein m is 1, 2, 3 or 4, and R^{A1} and R^{A2} are each hydrogen or (1-2C)alkyl;

X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(N R^{A3})N(R^{A3})- or—S—, wherein each R^{A3} is independently hydrogen or methyl; and

Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl, wherein each (1-6C)alkyl, (2-6C)alkenyl, (2-6C)alkynyl, aryl, (3-6C)cycloalkyl, (3-6C)cycloalkenyl, and heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{A4} R^{A5}, and (1-4C)alkoxy, wherein RM and R^{A5} are each independently hydrogen or (1-2C)alkyl.

33. The method of paragraph 30 or 31, wherein each R¹ is independently —Y¹ —X¹ —Z¹, wherein:

Y¹ is absent or —(CR^{A1}R^{A2})ₘ—, wherein m is 1, 2, 3, or 4, and R^{A1} and R″ are each independently hydrogen or (1-2C)alkyl;

X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(NR^{A3})N(R^{A3})—, or—S—, wherein each R^{A3} is independently hydrogen or methyl; and

Z¹ is a further oligonucleotide or is hydrogen, (1-6C)alkyl, aryl, (3-6C)cycloalkyl, or heteroaryl, wherein each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

34. The method of paragraph 30 or 31, wherein each R¹ is independently —Y¹—X¹—Z¹, wherein:

Y¹ is absent or a group of the formula —(cRA1RA2)ₘ—, wherein m is 1, 2, 3 or 4, and R^{A1} and R^{A2} are each independently hydrogen or (1-2C)alkyl;

X¹ is absent, —C(O)—, —C(O)O—, —N(R^{A3})—C(O)—, —C(O)—N(R^{A3})—, wherein each R^{A3} is hydrogen or methyl; and

Z¹ is a further oligonucleotide or is hydrogen, (1- 6C)alkyl, aryl, (3-6C)cycloalkyl, or heteroaryl, wherein each (1-6C)alkyl, aryl, (3- 6C)cycloalkyl, and heteroaryl is optionally substituted by one or more (e.g., 1, 2, 3, 4, or 5) substituent groups selected from the group consisting of (1-4C) alkyl, halo, and hydroxy.

35. The method of paragraph 30 or 31, wherein each R¹ is independently —Y¹—X¹—Z¹, wherein:

Y¹ is absent, —(CH₂)—, or —(CH₂CH₂)—;

X¹ is absent, —N(R^{A3})—C(O)—, —C(O)—N(R^{A3})—, wherein each R^{A3} is independently hydrogen or methyl; and

Z¹ is hydrogen or (1-2C)alkyl.

36. The method of any one of paragraphs 30 to 35, wherein each R² is independently —Y²—Z²,

wherein Y² is absent or —(CR^{B1} R^{B2})m—, wherein m is 1, 2, 3 or 4, and R^{B1} and R^{B2} are each independently hydrogen or (1-2C)alkyl; and

Z² is hydrogen or (1-6C)alkyl.

37. The method of any one of paragraphs 30 to 36, wherein each R² is hydrogen. 38. The method of any one of paragraphs 30 to 37, wherein n is 2 or 3.

39. The method of any one of paragraphs 30 to 37, wherein n is 1.

40. The method of any one of paragraphs 1 to 39, wherein the linker is an amino acid residue selected from the group consisting of glutamic acid, succinic acid, and gamma-aminobutyric acid residues.

41. The method of any one of paragraphs 1 to 39, wherein the linker is of the following structure:

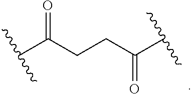 [SMILES: *C(=O)CCC(*)=O]

42. The method of any one of paragraphs 1 to 39, wherein the linker is of the following structure:

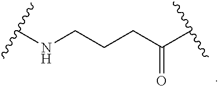 [SMILES: *NCCCC(*)=O]

43. The method of any one of paragraphs 1 to 39, wherein the linker is of the following structure:

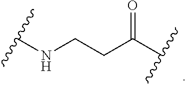 [SMILES: *NCCC(*)=O]

44. The method of any one of paragraphs 1 to 39, wherein the linker is of the following structure:

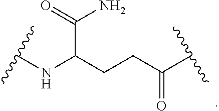 [SMILES: *NC(CCC(*)=O)C(N)=O]

45. The method of any one of paragraphs 1 to 39, wherein the linker is of the following structure:

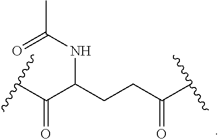 [SMILES: *C(=O)CCC(NC(C)=O)C(*)=O]

46. The method of any one of paragraphs 1 to 39, wherein the conjugate comprises or is of the following structure:

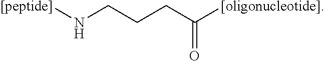

47. The method of any one of paragraphs 1 to 39, wherein the conjugate comprises or is of the following structure:

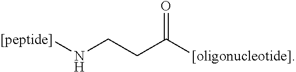

48. The method of any one of paragraphs 1 to 39, wherein the conjugate comprises or is of the following structure:

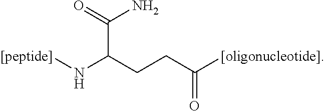

49. The method of any one of paragraphs 1 to 39, wherein the conjugate comprises or is of the following structure:

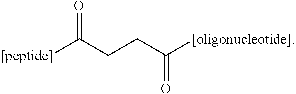

50. The method of any one of paragraphs 1 to 39, wherein the conjugate comprises or is of the following structure:

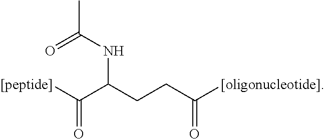

51. The method of any one of paragraphs 1 to 50, wherein the oligonucleotide is bonded to the linker or the peptide at its 3′ terminus.

52. The method of paragraph 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), ora thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂.

53. The method of paragraph 52, wherein the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYBRBR, wherein free —COOH, if any, in the glutamic acid residue is replaced with —CONH₂.

54. The method of paragraph 52, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂.

55. The method of paragraph 52, wherein the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂.

56. The method of paragraph 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂.

57. The method of paragraph 56, wherein the conjugate comprises or consists of 5′-CTCCAACATCAAGGAAGATGGCATTTCTAG-3′ (SEQ ID NO: 106) having a 3′-terminus covalently linked via a glutamic acid residue to N-terminus of peptide RBRRBRFQILYBRBR—NH₂, wherein free —COOH, if any, in the glutamic acid residue is replaced with —CONH₂.

58. The method of paragraph 56, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂.

59. The method of paragraph 56, wherein the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂.

60. The method of paragraph 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via gamma-aminobutyric acid residue to C-terminus of peptide Ac-RBRRBRFQILYRBHBH.

61. The method of paragraph 60, wherein the conjugate comprises or consists of 5′-CTCCAACATCAAGGAAGATGGCATTTCTAG-3′ (SEQ ID NO: 106) having a 3′-terminus covalently linked via gamma-aminobutyric acid residue to C-terminus of peptide Ac-RBRRBRFQILYRBHBH.

62. The method of paragraph 60, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via gamma-aminobutyric acid residue to C-terminus of peptide Ac-RBRRBRFQILYRBHBH.

63. The method of paragraph 60, wherein the conjugate comprises or consists of 5′-CACCCACCAUCACCCUCUGUG-3′ (SEQ ID NO: 115), or a thymine-substitution analogue thereof, having a 3′-terminus covalently linked via gamma-aminobutyric acid residue to C-terminus of peptide Ac-RBRRBRFQILYRBHBH.

64. The method of paragraph 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to C-terminus of peptide Ac-RBRRBRFQILYRBHBH, wherein free —COOH, if any, in the glutamic acid residue is replaced with —CONH₂.

65. The method of paragraph 64, wherein the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYRBHBH, wherein free —COOH, if any, in the glutamic acid residue is replaced with —CONH₂.

66. The method of paragraph 64, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to C-terminus of peptide Ac-RBRRBRFQILYRBHBH, wherein free —COOH, if any, in the glutamic acid residue is replaced with —CONH_{2.}

67. The method of paragraph 64, wherein the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to C-terminus of peptide Ac-RBRRBRFQILYRBHBH, wherein free —COOH, if any, in the glutamic acid residue is replaced with —CONH₂.

68. The method of any one of paragraphs 52 to 59 and 64 to 67, wherein the linker is of the following structure:

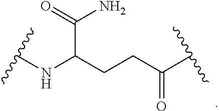 [SMILES: *NC(CCC(*)=O)C(N)=O]

69. The method of paragraph 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a beta-alanine residue to C-terminus of peptide Ac-RBRRBRFQILYBRBR.

70. The method of paragraph 69, wherein the conjugate comprises or consists of 5′-CTCCAACATCAAGGAAGATGGCATTTCTAG-3′ (SEQ ID NO: 106) having a 3′-terminus covalently linked via a beta-alanine residue to C-terminus of peptide Ac-RBRRBRFQILYBRBR.

71. The method of paragraph 69, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a beta-alanine residue to C-terminus of peptide Ac-RBRRBRFQILYBRBR.

72. The method of paragraph 69, wherein the conjugate comprises or consists of 5′-CACCCACCAUCACCCUCUGUG-3′ (SEQ ID NO: 115), or a thymine-substitution analogue thereof, having a 3′-terminus covalently linked via a beta-alanine residue to C-terminus of peptide Ac-RBRRBRFQILYBRBR.

73. The method of paragraph 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-20 ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a beta-alanine residue to C-terminus of peptide Ac-RBRRBRFQILYRBHBH.

74. The method of paragraph 73, wherein the conjugate comprises or consists of 5′-CTCCAACATCAAGGAAGATGGCATTTCTAG-3′ (SEQ ID NO: 106) having a 3′-terminus covalently linked via a beta-alanine residue to C-terminus of peptide Ac-RBRRBRFQILYRBHBH.

75. The method of paragraph 73, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a beta-alanine residue to C-terminus of peptide Ac-RBRRBRFQILYRBHBH.

76. The method of paragraph 73, wherein the conjugate comprises or consists of 5′-CACCCACCAUCACCCUCUGUG-3′ (SEQ ID NO: 115), or a thymine-substitution analogue thereof, having a 3′-terminus covalently linked via a beta-alanine residue to C-terminus of peptide Ac-RBRRBRFQILYRBHBH.

77. The method of any one of paragraphs 1 to 76, wherein the oligonucleotide is a morpholino.

78. The method of paragraph 77, wherein all morpholino internucleoside linkages are —P(O)(NMe₂)O—.

79. The method of any paragraph 78, wherein the oligonucleotide comprises the following group as its 5′ terminus:

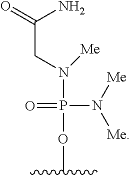 [SMILES: *OP(=O)(N(C)C)N(C)CC(N)=O]

80. The method of any one of paragraphs 1 to 78, wherein the oligonucleotide comprises the following group as its 5′ terminus:

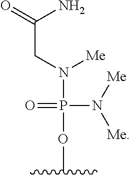 [SMILES: *OP(=O)(N(C)C)N(C)CC(N)=O]

81. The method of any preceding paragraph, wherein the conjugate is administered parenterally.

82. The method of paragraph 81, wherein the conjugate is administered by infusion.

83. The method of paragraph 82, wherein the conjugate is administered by intravenous infusion.

84. The method of any preceding paragraph, wherein the subject is amenable to exon 51 skipping.

85. The method of any preceding paragraph, wherein the conjugate is administered to the subject at a frequency that is weekly to quarterly.

86. The method of any preceding paragraph, wherein the conjugate is administered to the subject at a frequency that is weekly to monthly.

87. The method of paragraph 86, wherein the frequency is weekly, biweekly, or monthly.

88. The method of any one of paragraphs 1 to 84, wherein the frequency is quarterly.

89. The method of any preceding paragraph, wherein 40 mg/kg to 60 mg/kg, 30 mg/kg to 50 mg/kg, 30 mg/kg to 40 mg/kg, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, 45 mg/kg to 60 mg/kg, 1 mg/kg to 30 mg/kg, 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of conjugate is administered.

90. The method of paragraph any preceding paragraph, wherein 1 mg/kg, 4 mg/kg, 5 mg/kg, 6 mg/kg, 8 mg/kg, 10 mg/kg, 15 mg/kg, 20 mg/kg, 25 mg/kg, 30 mg/kg, 35 mg/kg, 40 mg/kg, 45 mg/kg, 50 30 mg/kg, or 60 mg/kg of conjugate is administered.

91. The method of paragraph 90, wherein 1 mg/kg, 5 mg/kg, 10 mg/kg, 15 mg/kg, or 20 mg/kg of conjugate is administered.

92. The method of any preceding paragraph, wherein the method is for use in treating a cardiac effect of DMD including, e.g., cardiomyopathy (e.g., dilated, hypertrophic, or restrictive cardiomyopathy), heart failure, and/or a cardiac arrhythmia.

Other embodiments are within the scope of the claims.

What is claimed is:

1. A method of treating a subject having Duchenne muscular dystrophy, the method comprising administering 1 mg/kg to 60 mg/kg of a conjugate of an oligonucleotide and a peptide covalently bonded or linked via a linker to the oligonucleotide,
the peptide comprising at least one cationic domain comprising at least 4 amino acid residues and at least one hydrophobic domain comprising at least 3 amino acid residues, provided that the peptide comprises a total of 7 to 40 amino acid residues, and provided that the at least one cationic domain comprises a beta-alanine residue in combination with arginine and/or histidine residues; and
the oligonucleotide comprising a total of 12 to 40 contiguous nucleobases, wherein at least 12 contiguous nucleobases are complementary to a target sequence in a human dystrophin gene.

2. The method of claim 1, wherein the oligonucleotide comprises a sequence selected from the group consisting of:
(SEQ ID NO: 106)
5′-CTCCAACATCAAGGAAGATGGCATTTCTAG-3′;
or
(SEQ ID NO: 107)
5′-ACCAGAGUAACAGUCUGAGUAGGAGC-3′;
(SEQ ID NO: 108;
5′-CUCAUACCUUCUGCUUGAUGAUC-3′;
(SEQ ID NO: 109)
5′-UUCUGUCCAAGCCCGGUUGAAAUC-3′;
(SEQ ID NO: 110)
5′-ACAUCAAGGAAGAUGGCAUUUCUAGUUUGG-3′;
(SEQ ID NO: 111)
5′-ACAUCAAGGAAGAUGGCAUUUCUAG-3′;
(SEQ ID NO: 112)
5′-CUCCAACAUCAAGGAAGAUGGCAUUUCUAG-3′;
(SEQ ID NO: 113)
5′-AUCAUUUUUUCUCAUACCUUCUGCUAG-3′;
(SEQ ID NO: 114)
5′-AUCAUUUUUUCUCAUACCUUCUGCUAGGAGCUAAAAAG-3′;
(SEQ ID NO: 115)
5′-CACCCACCAUCACCCUCUGUG-3′;
(SEQ ID NO: 116)
5′-AUCAUCUCGUUGAUAUCCUCAA-3′;
and their thymine-substitution analogues

3. The method of claim 1, wherein each cationic domain has length of between 4 and 12 amino acid residues.

4. The method of claim 3, wherein each cationic domain has length of between 4 and 7 amino acid residues.

5. The method of any one of claims 1-4, wherein each cationic domain comprises at least 55%, at least 60%, at least 65% at least 70%, at least 75%, at least 80%, at least 85%, at least 90%, or at least 95% cationic amino acids.

6. The method of any one of claims 1-4, wherein each cationic domain comprises at least 40%, at least 45%, at least 50%, at least 55%, at least 60%, at least 60%, at least 65%, at least 70% arginine and/or histidine residues.

7. The method of any one of claims 1-4, wherein each cationic domain comprises one of the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19) or any combination thereof.

8. The method of any one of claims 1-4, wherein each cationic domain comprises or consists of one the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO:

11. , RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19) or any combination thereof.

9. The method of any one of claims 1-4, wherein the peptide comprises two cationic domains.

10. The method of any one of claims 1-4, wherein each hydrophobic domain has a length of 3 to 6 amino acids, preferably each hydrophobic domain has a length of 5 amino acids.

11. The method of any one of claims 1-4, wherein each hydrophobic domain comprises at least 70%, at least 75%, at least 80%, at least 85%, at least 90%, at least 95%, or 100% hydrophobic amino acids.

12. The method of any one of claims 1-4, wherein each hydrophobic domain comprises phenylalanine, leucine, Isoleucine, tyrosine, tryptophan, proline, and/or glutamine residues; preferably wherein each hydrophobic domain comprises or consists of phenylalanine, leucine, isoleucine, tyrosine, tryptophan, proline, and/or glutamine residues.

13. The method of any one of claims 1-4, wherein the peptide comprises one hydrophobic domain.

14. The method of any one of claims 1-4, wherein the or each hydrophobic domain comprises one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26) or any combination thereof.

15. The method of any one of claims 1-4, wherein the or each hydrophobic domain comprises or consists of one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26) or any combination thereof.

16. The method of any one of claims 1-4, wherein the peptide comprises or consists of two cationic domains and one hydrophobic domain.

17. The method of any one of claims 1-4, wherein the peptide comprises or consists of one hydrophobic core domain flanked by two cationic arm domains.

18. The method of any one of claims 1-4, wherein the peptide comprises or consists of one hydrophobic core domain comprising a sequence selected from: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26), flanked by two cationic arm domains each comprising a sequence selected from: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO:

11. , RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19).

19. The method of any one of claims 1-4, wherein the peptide comprises or consists of one of the following sequences: RBRRBRRFQILYRBRBR (SEQ ID NO: 27), RBRRBRRYQFLIRBRBR (SEQ ID NO:

31. , RBRRBRRILFQYRBRBR (SEQ ID NO: 32), RBRRBRFQILYBRBR (SEQ ID NO: 35), RBRRBRRFQILYRBHBH (SEQ ID NO: 37), RBRRBRRFQILYHBHBR (SEQ ID NO: 38), and RBRRBRFQILYRBHBH (SEQ ID NO: 44).

20. The method of any one of claims 1-4, wherein the peptide has the following amino acid sequence RBRRBRFQILYBRBR (SEQ ID NO: 35).

21. The method of any one of claims 1-4, wherein the peptide has the following amino acid sequence RBRRBRRFQILYRBHBH (SEQ ID NO: 37).

22. The method of any one of claims 1-4, wherein the peptide has the following amino acid sequence RBRRBRFQILYRBHBH (SEQ ID NO: 44).

23. The method of any one of claims 1-4, wherein the peptide is bonded to the rest of the conjugate through its N-terminus.

24. The method of claim 23, wherein the C-terminus of the peptide is —NH₂.

25. The method of any one of claims 1-4, wherein the peptide is bonded to the rest of the conjugate through its C-terminus.

26. The method of claim 25, wherein the peptide is acylated at its N-terminus.

27. The method of any one of claims 1-4, wherein the conjugate comprises or is of the following structure:
[peptide]-[linker]-[oligonucleotide]

28. The method of any one of claims 1-4, wherein the conjugate comprises or is of the following structure:

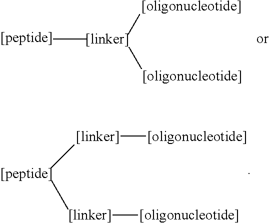

29. The method of any one of claims 1-4, wherein the conjugate comprises or is of the following structure:
[peptide]-[linker]-[peptide]-[linker]-[oligonucleotide].

30. The method of any one of claims 1-4, wherein each linker is independently of formula (I):
T₁—(CR¹R²)ₙ—T₂. (I)
wherein
T₁ is a divalent group for attachment to the peptide and is selected from the group consisting of —NH— and carbonyl;
T₂ is a divalent group for attachment to an oligonucleotide and is selected from the group consisting of —NH- and carbonyl;
n is 1, 2 or 3;
each R¹ is independently —Y¹ —X¹ —Z¹,
wherein
Y¹ is absent or —(CR^{A1}R^{A2})ₘ—, wherein m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —N(R^{A3})C(N R^{A3})N(R^{A3})—, —SO—, —S—, —SO₂—, —S(O)₂N(R^{A3})—, or —N(R^{A3})SO₂—, wherein each R^{A3} is independently selected from hydrogen and methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl,
wherein each (1-6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4} R^{A5}, and (1-4C)alkoxy, wherein R^{A4} and R^{A5} are each independently selected from the group consisting of hydrogen and (1-4C)alkyl; and
each R² is independently —Y²—X²—Z², wherein
Y² is absent or a group of the formula —[CR^{B1} R^{B2}]ₙ— in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})—, or —N(R^{B3})—SO₂—, wherein each R^{B3} is independently selected from hydrogen or methyl; and
Z² is selected from hydrogen, (1-6C)alkyl, (2-6C)alkenyl, (2-6C)alkynyl, aryl, (3-6C)cycloalkyl, (3-6C)cycloalkenyl or heteroaryl, wherein each (1-6C)alkyl, (2- 6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4} R^{B5}, and (1-4C)alkoxy, wherein R^{B4} and R^{B5} are each independently hydrogen or (1-2C)alkyl; with the proviso that; when n=1 and T₁ and T₂ are different to one another, then R¹ and R² are not both H; when n=1, T₁ and T₂ are different to one another and one of R¹ and R² is H then the other of R¹ and R² is not methyl; or when n=2 and each occurrence of R¹ and R² is H, then T₁ and T₂ are both —C(O)- or are both —NH—.

31. The method of claim 30, wherein T₂ is —C(O)—.

32. The method of claim 30, wherein each R¹ is independently —Y¹ —X¹ —Z¹, wherein:
Y¹is absent or —(CR^{A1}R^{A2})ₘ—, wherein m is 1, 2, 3 or 4, and R^{A1} and R^{A2} are each hydrogen or (1-2C)alkyl;
X¹ is absent, —O—, —C(O)- 7 —C(O)O—, —N(R^{A3})—, —N(R^{A3})—C(O)—, —C(O)—N(R^{A3})—, —N(R^{A3})C(O)N(R^{A3})—, —N(R^{A3})C(N R^{A3})N(R^{A3})- or—S—, wherein each R^{A3} is independently hydrogen or methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl, wherein each (1-6C)alkyl, (2-6C)alkenyl, (2-6C)alkynyl, aryl, (3-6C)cycloalkyl, (3-6C)cycloalkenyl, and heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{A4} R^{A5}, and (1-4C)alkoxy, wherein RM and R^{A5} are each independently hydrogen or (1-2C)alkyl.

33. The method of claim 30, wherein each R¹ is independently —Y¹ —X¹ —Z¹, wherein:
Y¹is absent or —(CR^{A1}R^{A2})ₘ—, wherein m is 1, 2, 3, or 4, and R^{A1} and R″ are each independently hydrogen or (1-2C)alkyl;
X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(NR^{A3})N(R^{A3})—, or—S—, wherein each R^{A3} is independently hydrogen or methyl; and
Z¹ is a further oligonucleotide or is hydrogen, (1-6C)alkyl, aryl, (3-6C)cycloalkyl, or heteroaryl, wherein each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

34. The method of claim 30, wherein each R¹ is independently 7 wherein:
Y¹ is absent or a group of the formula —(cRA1RA2)ₘ—, wherein m is 1, 2, 3 or 4, and R^{A1} and R^{A2} are each independently hydrogen or (1-2C)alkyl;
X¹ is absent, —C(O)—, —C(O)O—, —N(R^{A3})—C(O)—, —C(O)—N(R^{A3})—, wherein each R^{A3} is hydrogen or methyl; and
Z¹ is a further oligonucleotide or is hydrogen, (1- 6C)alkyl, aryl, (3-6C)cycloalkyl, or heteroaryl, wherein each (1-6C)alkyl, aryl, (3- 6C)cycloalkyl, and heteroaryl is optionally substituted by one or more (e.g., 1, 2, 3, 4, or 5) substituent groups selected from the group consisting of (1-4C) alkyl, halo, and hydroxy.

35. The method of claim 30, wherein each R¹ is independently —Y¹—X¹—Z¹, wherein:
Y¹ is absent, —(CH₂)—, or —(CH₂CH₂)—;
X¹ is absent, —N(R^{A3})—C(O)—, —C(O)—N(R^{A3})—, wherein each R^{A3} is independently hydrogen or methyl; and
Z¹ is hydrogen or (1-2C)alkyl.

36. The method of claim 30, wherein each R² is independently —Y²—Z²,
wherein Y² is absent or —(CR^{B1} R^{B2} R^{B2}) m—, wherein m is 1, 2, 3 or 4, and R^{B1} and R^{B2} are each independently hydrogen or (1-2C)alkyl; and
Z² is hydrogen or (1-6C)alkyl.

37. The method of claim 30, wherein each R² is hydrogen.

38. The method of claim 30, wherein n is 2 or 3.

39. The method of claim 30, wherein n is 1.

40. The method of any one of claims 1-4, wherein the linker is an amino acid residue selected from the group consisting of glutamic acid, succinic acid, and gamma-aminobutyric acid residues.

41. The method of any one of claims 1-4, wherein the linker is of the following structure:

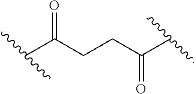

42. The method of any one of claims 1-4, wherein the linker is of the following structure:

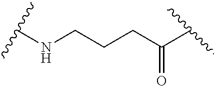

43. The method of any one of claims 1-4, wherein the linker is of the following structure:

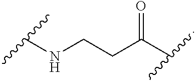

44. The method of any one of claims 1-4, wherein the linker is of the following structure:

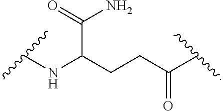

45. The method of any one of claims 1-4, wherein the linker is of the following structure:

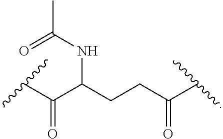

46. The method of any one of claims 1-4, wherein the conjugate comprises or is of the following structure:

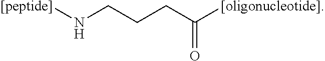

47. The method of any one of claims 1-4, wherein the conjugate comprises or is of the following structure:

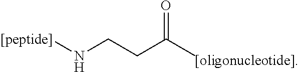

48. The method of any one of claims 1-4, wherein the conjugate comprises or is of the following structure:

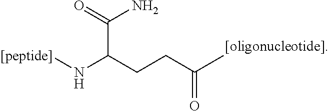

49. The method of any one of claims 1-4, wherein the conjugate comprises or is of the following structure:

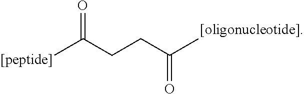

50. The method of any one of claims 1-4, wherein the conjugate comprises or is of the following structure:

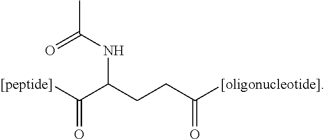

51. The method of any one of claims 1-4, wherein the oligonucleotide is bonded to the linker or the peptide at its 3′ terminus.

52. The method of claim 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂.

53. The method of claim 52, wherein the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYBRBR, wherein free —COOH, if any, in the glutamic acid residue is replaced with —CONH₂.

54. The method of claim 52, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂.

55. The method of claim 52, wherein the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to C-terminus of peptide Ac-RBRRBRFQILYBRBR, wherein free —COOH, if any, in the glutamic acid residue is replaced with —CONH₂.

56. The method of claim 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂.

57. The method of claim 56, wherein the conjugate comprises or consists of 5′-CTCCAACATCAAGGAAGATGGCATTTCTAG-3′ (SEQ ID NO: 106) having a 3′-terminus covalently linked via a glutamic acid residue to N-terminus of peptide RBRRBRFQILYBRBR—NH₂, wherein free —COOH, if any, in the glutamic acid residue is replaced with —CONH₂.

58. The method of claim 56, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂.

59. The method of claim 56, wherein the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to N-terminus of peptide RBRRBRFQILYBRBR—NH₂, wherein free —COOH, if any, in the glutamic acid residue is replaced with —CONH₂.

60. The method of claim 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via gamma-aminobutyric acid residue to C-terminus of peptide Ac-RBRRBRFQILYRBHBH.

61. The method of claim 60, wherein the conjugate comprises or consists of 5′-CTCCAACATCAAGGAAGATGGCATTTCTAG-3′ (SEQ ID NO: 106) having a 3′-terminus covalently linked via gamma-aminobutyric acid residue to C-terminus of peptide Ac-RBRRBRFQILYRBHBH.

62. The method of claim 60, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via gamma-aminobutyric acid residue to C-terminus of peptide Ac-RBRRBRFQILYRBHBH.

63. The method of claim 60, wherein the conjugate comprises or consists of 5′-CACCCACCAUCACCCUCUGUG-3′ (SEQ ID NO: 115), or a thymine-substitution analogue thereof, having a 3′-terminus covalently linked via gamma-aminobutyric acid residue to C-terminus of peptide Ac-RBRRBRFQILYRBHBH.

64. The method of claim 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a glutamic acid residue to C-terminus of peptide Ac-RBRRBRFQILYRBHBH, wherein free —COOH, if any, in the glutamic acid residue is replaced with —CONH₂.

65. The method of claim 64, wherein the conjugate comprises or consists of 5′-CTCCAACATCAAGGAAGATGGCATTTCTAG-3′ (SEQ ID NO: 106) having a 3′-terminus covalently linked via a glutamic acid residue to C-terminus of peptide Ac-RBRRBRFQILYRBHBH, wherein free —COOH, if any, in the glutamic acid residue is replaced with —CONH₂.

66. The method of claim 64, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a glutamic acid residue to C-terminus of peptide Ac-RBRRBRFQILYRBHBH, wherein free —COOH, if any, in the glutamic acid residue is replaced with —CONH₂.

67. The method of claim 64, wherein the conjugate comprises or consists of 5′-CACCCACCAUCACCCUCUGUG-3′ (SEQ ID NO: 115), or a thymine-substitution analogue thereof, having a 3′-terminus covalently linked via a glutamic acid residue to C-terminus of peptide Ac-RBRRBRFQILYRBHBH, wherein free —COON, if any, in the glutamic acid residue is replaced with —CONH₂.

68. The method of any one of claim 1-4 or 52-67, wherein the linker is of the following structure:

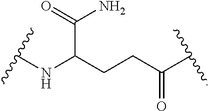

69. The method of claim 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a beta-alanine residue to C-terminus of peptide Ac-RBRRBRFQILYBRBR.

70. The method of claim 69, wherein the conjugate comprises or consists of 5′-CTCCAACATCAAGGAAGATGGCATTTCTAG-3′ (SEQ ID NO: 106) having a 3′-terminus covalently linked via a beta-alanine residue to C-terminus of peptide Ac-RBRRBRFQILYBRBR.

71. The method of claim 69, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a beta-alanine residue to C-terminus of peptide Ac-RBRRBRFQILYBRBR.

72. The method of claim 69, wherein the conjugate comprises or consists of 5′-CACCCACCAUCACCCUCUGUG-3′ (SEQ ID NO: 115), or a thymine-substitution analogue thereof, having a 3′-terminus covalently linked via a beta-alanine residue to C-terminus of peptide Ac-RBRRBRFQILYBRBR.

73. The method of claim 1, wherein the conjugate comprises or consists of 5′-CTCCAACATCAAGGAAGATGGCATTTCTAG-3′ (SEQ ID NO: 106), 5′-ACCAGAGUAACAGUCUGAGUAGGAGC-3′ (SEQ ID NO: 107), 5′-CUCAUACCUUCUGCUUGAUGAUC-3′ (SEQ ID NO: 108), 5′-UUCUGUCCAAGCCCGGUUGAAAUC-3′ (SEQ ID NO: 109), 5′-ACAUCAAGGAAGAUGGCAUUUCUAGUUUGG-3′ (SEQ ID NO: 110), 5′-ACAUCAAGGAAGAUGGCAUUUCUAG-3′ (SEQ ID NO: 111), 5′-CUCCAACAUCAAGGAAGAUGGCAUUUCUAG-3′ (SEQ ID NO: 112), 5′-AUCAUUUUUUCUCAUACCUUCUGCUAG-3′ (SEQ ID NO: 113), 5′-AUCAUUUUUUCUCAUACCUUCUGCUAGGAGCUAAAAAG-3′ (SEQ ID NO: 114), 5′-CACCCACCAUCACCCUCUGUG-3′ (SEQ ID NO: 115), or 5′-AUCAUCUCGUUGAUAUCCUCAA-3′ (SEQ ID NO: 116), or a thymine-substitution analogue thereof, having a 3′-terminus covalently linked via a beta-alanine residue to C-terminus of peptide Ac-RBRRBRFQILYRBHBH.

74. The method of claim 73, wherein the conjugate comprises or consists of 5′-CTCCAACATCAAGGAAGATGGCATTTCTAG-3′ (SEQ ID NO: 106) having a 3′-terminus covalently linked via a beta-alanine residue to C-terminus of peptide Ac-RBRRBRFQILYRBHBH.

75. The method of claim 73, wherein the conjugate comprises or consists of 5′-UUCUGUCCAAGCCCGGUUGAAAUC-3′ (SEQ ID NO: 109), or a thymine-substitution analogue thereof, having a 3′-terminus covalently linked via a beta-alanine residue to C-terminus of peptide Ac-RBRRBRFQILYRBHBH.

76. The method of claim 73, wherein the conjugate comprises or consists of 5′-CACCCACCAUCACCCUCUGUG-3′ (SEQ ID NO: 115), or a thymine-substitution analogue thereof, having a 3′-terminus covalently linked via a beta-alanine residue to C-terminus of peptide Ac-RBRRBRFQILYRBHBH.

77. The method of any one of claims 1-4, 52-67, and 69-76, wherein the oligonucleotide is a morpholino.

78. The method of claim 77, wherein all morpholino internucleoside linkages are —P(O)(NMe₂)O—.

79. The method of claim 78, wherein the oligonucleotide comprises the following group as its 5′ terminus:

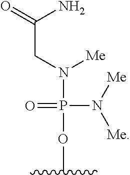 [SMILES: *OP(=O)(N(C)C)N(C)CC(N)=O]

80. The method of any one of claims 1-4, 52-67, and 69-76, wherein the oligonucleotide comprises the following group as its 5′ terminus:

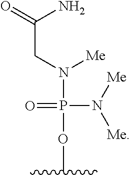 [SMILES: *OP(=O)(N(C)C)N(C)CC(N)=O]

81. The method of any one of claims 1-4, 52-67, and 69-76, wherein the conjugate is administered parenterally.

82. The method of claim 81, wherein the conjugate is administered by infusion.

83. The method of claim 82, wherein the conjugate is administered by intravenous infusion.

84. The method of any one of claims 1-4, 52-67, and 69-76, wherein the subject is amenable to exon 51 skipping.

85. The method of any one of claims 1-4, 52-67, and 69-76, wherein the conjugate is administered to the subject at a frequency that is weekly to quarterly.

86. The method of any one of claims 1-4, 52-67, and 69-76, wherein the conjugate is administered to the subject at a frequency that is weekly to monthly.

87. The method of claim 86, wherein the frequency is weekly, biweekly, or monthly.

88. The method of any one of claims 1-4, 52-67, and 69-76, wherein the frequency is quarterly.

89. The method of any one of claims 1-4, 52-67, and 69-76, wherein 40 mg/kg to 60 mg/kg, 30 mg/kg to 50 mg/kg, 30 mg/kg to 40 mg/kg, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, 45 mg/kg to 60 mg/kg, 1 mg/kg to 30 mg/kg, 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of conjugate is administered.

90. The method of claim any one of claims 1-4, 52-67, and 69-76, wherein 1 mg/kg, 4 mg/kg, 5 mg/kg, 6 mg/kg, 8 mg/kg, 10 mg/kg, 15 mg/kg, 20 mg/kg, 25 mg/kg, 30 mg/kg, 35 mg/kg, 40 mg/kg, 45 mg/kg, 50 mg/kg, or 60 mg/kg of conjugate is administered.

91. The method of claim 90, wherein 1 mg/kg, 5 mg/kg, 10 mg/kg, 15 mg/kg, or 20 mg/kg of conjugate is administered.

92. The method of any one of claims 1-4, 52-67, and 69-76, wherein the method is for use in treating a cardiac effect of DMD including, e.g., cardiomyopathy (e.g., dilated, hypertrophic, or restrictive cardiomyopathy), heart failure, and/or a cardiac arrhythmia.


SEQUENCE LISTING (122 sequences)

SEQ ID NO: 1 - protein, 7 aa, Artificial Sequence
  Synthetic Construct
  MISC_FEATURE (2)..(2): Xaa is bAla
  MISC_FEATURE (5)..(5): Xaa is bAla
RXRRXRR

SEQ ID NO: 2 - protein, 5 aa, Artificial Sequence
  Synthetic Construct
  MISC_FEATURE (2)..(2): Xaa is bAla
  MISC_FEATURE (4)..(4): Xaa is bAla
RXRXR

SEQ ID NO: 3 - protein, 4 aa, Artificial Sequence
  Synthetic Construct
  MISC_FEATURE (2)..(2): Xaa is bAla
RXRR

SEQ ID NO: 4 - protein, 6 aa, Artificial Sequence
  Synthetic Construct
  MISC_FEATURE (2)..(2): Xaa is bAla
  MISC_FEATURE (5)..(5): Xaa is bAla
RXRRXR

SEQ ID NO: 5 - protein, 6 aa, Artificial Sequence
  Synthetic Construct
  MISC_FEATURE (3)..(3): Xaa is bAla
  MISC_FEATURE (5)..(5): Xaa is bAla
RRXRXR

SEQ ID NO: 6 - protein, 5 aa, Artificial Sequence
  Synthetic Construct
  MISC_FEATURE (2)..(2): Xaa is bAla
  MISC_FEATURE (5)..(5): Xaa is bAla
RXRRX

SEQ ID NO: 7 - protein, 4 aa, Artificial Sequence
  Synthetic Construct
  MISC_FEATURE (1)..(1): Xaa is bAla
  MISC_FEATURE (3)..(3): Xaa is bAla
XRXR

SEQ ID NO: 8 - protein, 5 aa, Artificial Sequence
  Synthetic Construct
  MISC_FEATURE (2)..(2): Xaa is bAla
  MISC_FEATURE (4)..(4): Xaa is bAla
RXHXH

SEQ ID NO: 9 - protein, 5 aa, Artificial Sequence
  Synthetic Construct
  MISC_FEATURE (2)..(2): Xaa is bAla
  MISC_FEATURE (4)..(4): Xaa is bAla
HXHXR

SEQ ID NO: 10 - protein, 7 aa, Artificial Sequence
  Synthetic Construct
  MISC_FEATURE (2)..(2): Xaa is bAla
  MISC_FEATURE (5)..(5): Xaa is bAla
RXRHXHR

SEQ ID NO: 11 - protein, 7 aa, Artificial Sequence
  Synthetic Construct
  MISC_FEATURE (2)..(2): Xaa is bAla
  MISC_FEATURE (4)..(4): Xaa is bAla
  MISC_FEATURE (5)..(5): Xaa is bAla
RXRXXHR

SEQ ID NO: 12 - protein, 6 aa, Artificial Sequence
  Synthetic Construct
  MISC_FEATURE (2)..(2): Xaa is bAla
  MISC_FEATURE (5)..(5): Xaa is bAla
RXRRXH

SEQ ID NO: 13 - protein, 6 aa, Artificial Sequence
  Synthetic Construct
  MISC_FEATURE (2)..(2): Xaa is bAla
  MISC_FEATURE (5)..(5): Xaa is bAla
HXRRXR

SEQ ID NO: 14 - protein, 5 aa, Artificial Sequence
  Synthetic Construct
  MISC_FEATURE (2)..(2): Xaa is bAla
  MISC_FEATURE (4)..(4): Xaa is bAla
HXHXH

SEQ ID NO: 15 - protein, 4 aa, Artificial Sequence
  Synthetic Construct
  MISC_FEATURE (1)..(1): Xaa is bAla
  MISC_FEATURE (3)..(3): Xaa is bAla
XHXH

SEQ ID NO: 16 - protein, 5 aa, Artificial Sequence
  Synthetic Construct
  MISC_FEATURE (1)..(1): Xaa is bAla
  MISC_FEATURE (3)..(3): Xaa is bAla
  MISC_FEATURE (5)..(5): Xaa is bAla
XRXSX

SEQ ID NO: 17 - protein, 5 aa, Artificial Sequence
  Synthetic Construct
  MISC_FEATURE (1)..(1): Xaa is bAla
  MISC_FEATURE (3)..(3): Xaa is bAla
  MISC_FEATURE (4)..(4): Xaa is Hyp
  MISC_FEATURE (5)..(5): Xaa is bAla
XRXXX

SEQ ID NO: 18 - protein, 6 aa, Artificial Sequence
  Synthetic Construct
  MISC_FEATURE (2)..(2): Xaa is Hyp
  MISC_FEATURE (4)..(4): Xaa is Hyp
  MISC_FEATURE (6)..(6): Xaa is bAla
RXHXHX

SEQ ID NO: 19 - protein, 6 aa, Artificial Sequence
  Synthetic Construct
  MISC_FEATURE (2)..(2): Xaa is Hyp
  MISC_FEATURE (5)..(5): Xaa is Hyp
RXRRXR

SEQ ID NO: 20 - protein, 5 aa, Artificial Sequence
  Synthetic Construct
YQFLI

SEQ ID NO: 21 - protein, 5 aa, Artificial Sequence
  Synthetic Construct
FQILY

SEQ ID NO: 22 - protein, 5 aa, Artificial Sequence
  Synthetic Construct
ILFQY

SEQ ID NO: 23 - protein, 4 aa, Artificial Sequence
  Synthetic Construct
FQIY

SEQ ID NO: 24 - protein, 8 aa, Artificial Sequence
  Synthetic Construct
WWWWWPWW

SEQ ID NO: 25 - protein, 4 aa, Artificial Sequence
  Synthetic Construct
WPWW

SEQ ID NO: 26 - protein, 4 aa, Artificial Sequence
  Synthetic Construct
WWPW

SEQ ID NO: 27 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYRXRXR

SEQ ID NO: 28 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
RXRRXRRFQILYRXRR

SEQ ID NO: 29 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRFQILYRRXRXR

SEQ ID NO: 30 - protein, 17 aa, Artificial Sequence
  Synthetic Construct
  MISC_FEATURE (2)..(2): Xaa is bAla
  MISC_FEATURE (4)..(4): Xaa is bAla
  MISC_FEATURE (12)..(12): Xaa is bAla
  MISC_FEATURE (15)..(15): Xaa is bAla
RXRXRFQILYRXRRXRR

SEQ ID NO: 31 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRYQFLIRXRXR

SEQ ID NO: 32 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRILFQYRXRXR

SEQ ID NO: 33 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRFQILYRXRXR

SEQ ID NO: 34 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (15)..(15): Xaa is bAla
RXRRXFQILYRXRRXR

SEQ ID NO: 35 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXRXR

SEQ ID NO: 36 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXFQILYRXRXR

SEQ ID NO: 37 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYRXHXH

SEQ ID NO: 38 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYHXHXR

SEQ ID NO: 39 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYHXRXH

SEQ ID NO: 40 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRYQFLIRXHXH

SEQ ID NO: 41 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRILFQYRXHXH

SEQ ID NO: 42 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRHXHRFQILYRXRXR

SEQ ID NO: 43 - protein, 17 aa, Artificial Sequence
  Synthetic Construct
  MISC_FEATURE (2)..(2): Xaa is bAla
  MISC_FEATURE (4)..(4): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRXXHRFQILYRXHXH

SEQ ID NO: 44 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRFQILYRXHXH

SEQ ID NO: 45 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRFQILYHXHXH

SEQ ID NO: 46 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXHFQILYRXHXH

SEQ ID NO: 47 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
HXRRXRFQILYRXHXH

SEQ ID NO: 48 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXFQILYRXHXH

SEQ ID NO: 49 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXHXH

SEQ ID NO: 50 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRYQFLIHXHXH

SEQ ID NO: 51 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRILFQYHXHXH

SEQ ID NO: 52 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYHXHXH

SEQ ID NO: 53 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXRXS

SEQ ID NO: 54 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
  MISC_FEATURE (15)..(15): Xaa is Hyp
RXRRXRFQILYXRXX

SEQ ID NO: 55 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is Hyp
RXRRXRFQILYXRXR

SEQ ID NO: 56 - protein, 16 aa, Artificial Sequence
  Synthetic Construct
  MISC_FEATURE (3)..(3): Xaa is bAla
  MISC_FEATURE (6)..(6): Xaa is bAla
  MISC_FEATURE (13)..(13): Xaa is bAla
  MISC_FEATURE (15)..(15): Xaa is bAla
RRXRRXRFQILYXRXR

SEQ ID NO: 57 - protein, 15 aa, Artificial Sequence
  Synthetic Construct
  MISC_FEATURE (1)..(1): Xaa is bAla
  MISC_FEATURE (4)..(4): Xaa is bAla
  MISC_FEATURE (12)..(12): Xaa is bAla
  MISC_FEATURE (14)..(14): Xaa is bAla
XRRXRRFQILYXRXR

SEQ ID NO: 58 - protein, 13 aa, Artificial Sequence
  Synthetic Construct
  MISC_FEATURE (2)..(2): Xaa is bAla
  MISC_FEATURE (5)..(5): Xaa is bAla
  MISC_FEATURE (10)..(10): Xaa is bAla
  MISC_FEATURE (12)..(12): Xaa is bAla
RXRRXRWWWXRXR

SEQ ID NO: 59 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWWPWWXRXR

SEQ ID NO: 60 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWPWWXRXR

SEQ ID NO: 61 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWWPWXRXR

SEQ ID NO: 62 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRRWWWRXRXR

SEQ ID NO: 63 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRWWPWWRXRXR

SEQ ID NO: 64 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWPWWRXRXR

SEQ ID NO: 65 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWWPWRXRXR

SEQ ID NO: 66 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRFQILYXRXR

SEQ ID NO: 67 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
RXRRXRRFQILYRXR

SEQ ID NO: 68 - protein, 16 aa, Artificial Sequence
  Synthetic Construct
  MISC_FEATURE (1)..(1): Xaa is bAla
  MISC_FEATURE (3)..(3): Xaa is bAla
  MISC_FEATURE (5)..(5): Xaa is bAla
  MISC_FEATURE (12)..(12): Xaa is bAla
  MISC_FEATURE (15)..(15): Xaa is bAla
XRXRXWWPWWRXRRXR

SEQ ID NO: 69 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRFQILYXHXH

SEQ ID NO: 70 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRFQIYRXHXH

SEQ ID NO: 71 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXRXH

SEQ ID NO: 72 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is Hyp
  MISC_FEATURE (15)..(15): Xaa is Hyp
RXRRXRFQILYRXHXH

SEQ ID NO: 73 - protein, 16 aa, Artificial Sequence
  Synthetic Construct
  MISC_FEATURE (2)..(2): Xaa is Hyp
  MISC_FEATURE (5)..(5): Xaa is Hyp
  MISC_FEATURE (13)..(13): Xaa is bAla
  MISC_FEATURE (15)..(15): Xaa is bAla
RXRRXRFQILYRXHXH

SEQ ID NO: 74 - protein, 16 aa, Artificial Sequence
  Synthetic Construct
  MISC_FEATURE (2)..(2): Xaa is Hyp
  MISC_FEATURE (5)..(5): Xaa is Hyp
  MISC_FEATURE (13)..(13): Xaa is Hyp
  MISC_FEATURE (15)..(15): Xaa is Hyp
RXRRXRFQILYRXHXH

SEQ ID NO: 75 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWWWRXHXH

SEQ ID NO: 76 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWWPRXHXH

SEQ ID NO: 77 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRPWWRXHXH

SEQ ID NO: 78 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRWWPWWRXHXH

SEQ ID NO: 79 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWWPWRXHXH

SEQ ID NO: 80 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWPWWRXHXH

SEQ ID NO: 81 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRRWWWRXHXH

SEQ ID NO: 82 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRWWPWWRXHXH

SEQ ID NO: 83 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWPWWRXHXH

SEQ ID NO: 84 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWWPWRXHXH

SEQ ID NO: 85 - protein, 17 aa, Artificial Sequence
  Synthetic Construct
  MISC_FEATURE (3)..(3): Xaa is bAla
  MISC_FEATURE (6)..(6): Xaa is bAla
  MISC_FEATURE (14)..(14): Xaa is bAla
  MISC_FEATURE (16)..(16): Xaa is bAla
RRXRRXRFQILYRXHXH

SEQ ID NO: 86 - protein, 16 aa, Artificial Sequence
  Synthetic Construct
  MISC_FEATURE (1)..(1)
  MISC_FEATURE (4)..(4): Xaa is bAla
  MISC_FEATURE (13)..(13): Xaa is bAla
  MISC_FEATURE (15)..(15): Xaa is bAla
XRRXRRFQILYRXHXH

SEQ ID NO: 87 - protein, 16 aa, Artificial Sequence
  Synthetic Construct
  MISC_FEATURE (3)..(3): Xaa is bAla
  MISC_FEATURE (6)..(6): Xaa is bAla
  MISC_FEATURE (13)..(13): Xaa is bAla
  MISC_FEATURE (15)..(15): Xaa is bAla
RRXRRXRFQILYXHXH

SEQ ID NO: 88 - protein, 15 aa, Artificial Sequence
  Synthetic Construct
  MISC_FEATURE (1)..(1): Xaa is bAla
  MISC_FEATURE (4)..(4): Xaa is bAla
  MISC_FEATURE (12)..(12): Xaa is bAla
  MISC_FEATURE (14)..(14): Xaa is bAla
XRRXRRFQILYXHXH

SEQ ID NO: 89 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXHRFQILYRXHXH

SEQ ID NO: 90-100 - skipped (11 entries)

SEQ ID NO: 101 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is Hyp
  MISC_FEATURE (14)..(14): Xaa is Hyp
RXRRXRFQILYXRXR

SEQ ID NO: 102 - protein, 15 aa, Artificial Sequence
  Synthetic Construct
  MISC_FEATURE (2)..(2): Xaa is Hyp
  MISC_FEATURE (5)..(5): Xaa is Hyp
  MISC_FEATURE (12)..(12): Xaa is bAla
  MISC_FEATURE (14)..(14): Xaa is bAla
RXRRXRFQILYXRXR

SEQ ID NO: 103 - protein, 15 aa, Artificial Sequence
  Synthetic Construct
  MISC_FEATURE (2)..(2): Xaa is Hyp
  MISC_FEATURE (5)..(5): Xaa is Hyp
  MISC_FEATURE (12)..(12): Xaa is Hyp
  MISC_FEATURE (14)..(14): Xaa is Hyp
RXRRXRFQILYXRXR

SEQ ID NO: 104 - protein, 13 aa, Artificial Sequence
  Synthetic Construct
  MISC_FEATURE (2)..(2): Xaa is bAla
  MISC_FEATURE (5)..(5): Xaa is bAla
  MISC_FEATURE (10)..(10): Xaa is bAla
  MISC_FEATURE (12)..(12): Xaa is bAla
RXRRXRWWWXRXR

SEQ ID NO: 105 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWWPWWXRXR

SEQ ID NO: 106 - DNA, 30 bp, Artificial Sequence
  Synthetic Construct
ctccaacatcaaggaagatggcatttctag

SEQ ID NO: 107 - RNA, 26 nt, Artificial Sequence
  Synthetic Construct
accagaguaacagucugaguaggagc

SEQ ID NO: 108 - RNA, 23 nt, Artificial Sequence
  Synthetic Construct
cucauaccuucugcuugaugauc

SEQ ID NO: 109 - RNA, 24 nt, Artificial Sequence
  Synthetic Construct
uucuguccaagcccgguugaaauc

SEQ ID NO: 110 - RNA, 30 nt, Artificial Sequence
  Synthetic Construct
acaucaaggaagauggcauuucuaguuugg

SEQ ID NO: 111 - RNA, 25 nt, Artificial Sequence
  Synthetic Construct
acaucaaggaagauggcauuucuag

SEQ ID NO: 112 - RNA, 30 nt, Artificial Sequence
  Synthetic Construct
cuccaacaucaaggaagauggcauuucuag

SEQ ID NO: 113 - RNA, 27 nt, Artificial Sequence
  Synthetic Construct
aucauuuuuucucauaccuucugcuag

SEQ ID NO: 114 - RNA, 38 nt, Artificial Sequence
  Synthetic Construct
aucauuuuuucucauaccuucugcuaggagcuaaaaag

SEQ ID NO: 115 - RNA, 21 nt, Artificial Sequence
  Synthetic Construct
cacccaccaucacccucugug

SEQ ID NO: 116 - RNA, 22 nt, Artificial Sequence
  Synthetic Construct
aucaucucguugauauccucaa

SEQ ID NO: 117 - DNA, 20 bp, Artificial Sequence
  Synthetic Construct
tgctcctgtggctgtctcct

SEQ ID NO: 118 - DNA, 21 bp, Artificial Sequence
  Synthetic Construct
agcttggctctggcctgtcct

SEQ ID NO: 119 - DNA, 20 bp, Artificial Sequence
  Synthetic Construct
accagccactcagccagtga

SEQ ID NO: 120 - DNA, 23 bp, Artificial Sequence
  Synthetic Construct
gattgttctagcctcttgattgc

SEQ ID NO: 121 - DNA, 21 bp, Artificial Sequence
  Synthetic Construct
ccagccactcagccagtgaag

SEQ ID NO: 122 - DNA, 24 bp, Artificial Sequence
  Synthetic Construct
cgatccgtaatgattgttctagcc